(19) United States
(12) Patent Application Publication
(10) Pub. No.: US 2022/0017865 A1
(43) Pub. Date: Jan. 20, 2022

(54) THERAPEUTIC GENE EDITING FOR ELANE-ASSOCIATED DISEASE

(71) Applicants: THE CHILDREN'S MEDICAL CENTER CORPORATION, Boston, MA (US); UNIVERSITY OF MASSACHUSETTS, Boston, MA (US)

(72) Inventors: Josias Brito FRAZÃO, Boston, MA (US); Scot A. WOLFE, Boston, MA (US); Daniel E. BAUER, Boston, MA (US); Shuquan RAO, Boston, MA (US); Peter E. NEWBURGER, Boston, MA (US)

(73) Assignees: THE CHILDREN'S MEDICAL CENTER CORPORATION, Boston, MA (US); UNIVERSITY OF MASSACHUSETTS, Boston, MA (US)

(21) Appl. No.: 17/297,925

(22) PCT Filed: Nov. 27, 2019
(86) PCT No.: PCT/US2019/063578

§ 371 Date: May 27, 2021

Related U.S. Application Data
(60) Provisional application No. 62/773,397, filed on Nov. 30, 2018.

(51) Int. Cl. C12N 5/0787; C12N 15/90; C12N 15/113; C12N 9/22

(52) U.S. Cl. CPC C12N 5/0642; C12N 15/907; C12N 15/1137; C12N 9/22; C12N 2310/20; C12N 2501/22; C12N 2501/2306; C12N 2501/26; C12N 2510/00; C12N 2501/125

(57) ABSTRACT

Provided herein are reagents and methods for targeting the ELANE gene for inhibition. Further provided herein is a method for producing a progenitor cell or a population of progenitor cells having decreased ELANE mRNA or protein expression.

### CROSS-REFERENCE TO RELATED APPLICATION

This application is a 371 National Phase Entry of International Patent Application No. PCT/US2019/063578 filed on Nov. 27, 2019, which is an International Application which designated the U.S., and which claims the benefit under 35 U.S.C. § 119(e) of U.S. Provisional Application No. 62/773,397 filed on Nov. 30, 2018, the contents of which are incorporated herein by reference in their entireties.

### GOVERNMENT SUPPORT

This invention was made with Government support under Grant No. 5RO1GM115911 awarded by the National Institutes of Health. The Government has certain rights in the invention.

### SEQUENCE LISTING

The instant application contains a Sequence Listing which has been submitted electronically in ASCII format and is hereby incorporated by reference in its entirety. Said ASCII copy, created on May 11, 2021, is named 701039-094110WOPT_SL.txt and is 16,284,709 bytes in size.

### BACKGROUND

Neutrophils are the most abundant type of white blood cells in the majority of mammals. Neutrophils play an essential role in innate immune defense against invading pathogens and are among the primary mediators of inflammatory response. During the acute phase of inflammation, neutrophils are the first inflammatory cells to leave the vasculature and migrate toward sites of inflammation, following a gradient of inflammatory stimuli.

Neutrophil elastase (NE), a serine protease enoded by the ELANE gene, is found in neutrophils. When an immune response is initiatied, e.g., to fight an infection, neutrophils release neutrophil elastase such that the protein can modify the function of certain cells and proteins to fight the infection. In addition to their involvement in pathogen destruction and regulation of proinflammatory processes, extracellular NE is also believed to initiate and/or contribute to the development of other diseases including inflammatory and degenerative conditions including chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis and chronic kidney disease⁴⁻⁹.

COPD is a life-threatening lung disease that interferes with normal breathing⁹. The World Health Organization (WHO) estimates that 210 million people have COPD worldwide, and data from 2005 show that more than 3 million people died of COPD that year. The COPD inflammatory process is caused by an imbalance between protease and anti-protease may lead to tissue damage. The excess of NE resulting from this imbalance hydrolyzes elastin, the structural protein which gives the lungs their elasticity. Cleavage of other matrix proteins, as well as inflammatory mediators, cell receptors, and lung surfactant is also promoted by the excess of extracellular NE. Thus, it is important to develop therapeutic approaches to protect the lungs from NE-mediated tissue damage.

Mutations of the ELANE gene are also the most common cause of two main forms of hereditary neutropenia: cyclic and severe congenital neutropenia. Cyclic neutropenia (CN) and severe congenital neutropenia (SCN) are both disorders of neutrophil production that that are both characterized by abnormally low levels of neutrophils in the body. These severe blood disorders lead to life-threatening infections in infancy in the absence of specific therapy. Most patients respond to G-CSF treatment, requiring lifelong treatment with regular subcutaneous infusions. Nonetheless in these patients there remains a predisposition to myelodysplastic syndrome and acute myeloid leukemia. The only curative option currently is allogeneic hematopoietic stem cell transplant, which is limited by donor availability and immune incompatibility with risk of graft rejection and graft-versus-host-disease.

Most mutations of the ELANE gene are missense and a common feature of ELANE mutations is their dominant nature. Production of an abnormal neutrophil elastase protein results in neutropenia despite the presence of one intact ELANE allele. Frameshift mutations associated with SCN are restricted to the distal penultimate exon (exon 4) and final exon (exon 5), suggesting that these mutations may escape NMD. Similar to missense mutations, NMD-escape mutations would be expected to give rise to an abnormal neutrophil elastase protein. A leading hypothesis suggests that abnormal neutrophil elastase proteins cause an unfolded protein response that ultimately results in apoptosis of neutrophil precursors at or around the promyelocyte stage.

Genome editing of autologous hematopoietic stem cells (HSCs) is a promising strategy to enable cure of a variety of ELANE-associated diseases including inflammatory and degenerative conditions like COPD (including acquired disease or genetic alpha-1 antitrypsin deficiency), rheumatoid arthritis, cystic fibrosis, acute respiratory distress syndrome, chronic kidney disease⁴⁻⁹ and blood disorders like CN and SCN. Prior efforts to use Cas9 and other programmable nucleases to edit human hematopoietic stem and progenitor cells (HSPCs) have shown variable efficiency, genotoxicity, requirement for HSC selection or expansion prior to infusion into recipient host, and limited ability to support long-term multilineage reconstitution with persistence of edited alleles. Provided here is an improved method of genome editing of the ELANE gene, and the related reagents thereof. The improved method comprises conditions for Cas9:sgRNA ribonucleoprotein (RNP) electroporation of HSCs that induce highly efficient on-target editing, no off-target editing while lacking detectable genotoxicity.

### SUMMARY

Embodiments described herein are based in part to the discovery of the following discoveries.

One aspect provided herein is a modified synthetic nucleic acid comprising a nucleic acid sequence shown in Table 1, SEQ ID NOS: 1-374, wherein there is at least one chemical modification to a nucleotide in the nucleic acid molecule.

In one embodiment of any aspect described herein, the at least one chemical modification is located at one or more terminal nucleotides in nucleic acid molecule. Exemplary chemical modifications include 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′- terminal cap known from nature, an xRNA base (analogous to “xDNA” base), an yRNA base (analogous to “yDNA” base), a PEG substituent, and a conjugated linker to a dye or non-fluorescent label (or tag).

In one embodiment of any aspect described herein, the at least one chemical modification is located only at the 3′ end, or added only at the 5′ end, or added at both the 5′ and 3′ ends of the synthetic nucleic acid molecule.

In one embodiment of any aspect described herein, the at least one chemical modification is located to first three nucleotides and to the last three nucleotides of the synthetic nucleic acid molecule.

In one embodiment of any aspect described herein, the modified synthetic nucleic acid sequence further comprising a crRNA/tracrRNA sequence.

In one embodiment of any aspect described herein, the modified synthetic nucleic acid molecule is a single guide RNA (sgRNA).

In one embodiment of any aspect described herein, any modified synthetic nucleic acid described herein is for use in the ex vivo targeted genome editing of the ELANE gene in a progenitor cell purpose.

In one embodiment of any aspect described herein, any modified synthetic nucleic acid described herein is for use in an ex vivo method of producing a progenitor cell or a population of progenitor cells wherein the cells or the differentiated progeny therefrom have decreased ELANE mRNA or protein expression.

In one embodiment of any aspect described herein, any modified synthetic nucleic acid described herein is for use in an ex vivo method of producing an isolated genetic engineered human cell or a population of genetic engineered human cells having at least one genetic modification.

In one embodiment of any aspect described herein, the modified synthetic nucleic acid molecule is used in combination with a DNA-targeting endonuclease Cas (CRISPR-associated) protein in a ribonucleoprotein (RNP) complex. In one embodiment of any aspect described herein, the Cas protein is Cas 9.

In one embodiment of any aspect described herein, any of the RNP complexes described herein is used in the electroporation of cells.

In one embodiment of any aspect described herein, the step of electroporation is performed in a solution comprising glycerol.

In one embodiment of any aspect described herein, the isolated human cell is a hematopoietic. In one embodiment of any aspect described herein, the hematopoietic progenitor is a cell of the erythroid lineage.

In one embodiment of any aspect described herein, the isolated human cell is an induced pluripotent stem cell.

In one embodiment of any aspect described herein, the progenitor cell or acquires at least one genetic modification.

In one embodiment of any aspect described herein, the at least one genetic modification is a deletion, insertion or substitution of the genetic sequence of the cell.

Another aspect described herein provides a composition comprising any of the modified synthetic nucleic acid molecule. In one embodiment of any aspect described herein, the composition further comprising a DNA-targeting endonuclease Cas (CRISPR-associated) protein.

In one embodiment of any aspect described herein, any composition described herein is for use in an ex vivo method of producing a progenitor cell or a population of progenitor cells wherein the cells or the differentiated progeny therefrom have decreased ELANE mRNA or protein expression.

In one embodiment of any aspect described herein, any composition described herein is for use in an ex vivo method of producing an isolated genetic engineered human cell or a population of progenitor cells having at least one genetic modification.

In one embodiment of any aspect described herein, any composition described herein is used in the electroporation of cells.

In one embodiment of any aspect described herein, the step of electroporation is performed in a solution comprising glycerol.

Another aspect described herein provides a ribonucleoprotein (RNP) complex comprising a DNA-targeting endonuclease Cas (CRISPR-associated) protein and any modified synthetic nucleic acid molecule described herein.

In one embodiment of any aspect described herein, any RNP complex described herein is for use in an ex vivo method of producing a progenitor cell or a population of progenitor cell wherein the cells or the differentiated progeny thereof have decreased ELANE mRNA or protein expression.

In one embodiment of any aspect described herein, any RNP complex described herein is for use in an ex vivo method of producing an isolated genetic engineered human cell or a population of genetic engineered human cells having at least one genetic modification.

In one embodiment of any aspect described herein, any RNP complex described herein is used in the electroporation of cells.

Yet another aspect described herein provides a method for producing a progenitor cell or a population of progenitor cells having decreased ELANE mRNA or protein expression, the method comprising contacting an isolated progenitor cell with an effective amount of any modified synthetic nucleic acid, composition, or ribonucleoprotein (RNP) complex described herein, whereby the contacted cells or the differentiated progeny cells therefrom have decreased ELANE mRNA or protein expression.

In one embodiment of any aspect described herein, the isolated progenitor cell is contacted ex vivo or in vitro.

In one embodiment of any aspect described herein, the contacted progenitor cell acquires at least one genetic modification.

In one embodiment of any aspect described herein, the contacted progenitor cell or contacted cell are further electroporated.

Another aspect described herein provides a population of genetically edited human cells having at least one genetic modification resulting in decreased ELANE mRNA or protein expression.

In one embodiment of any aspect described herein, the genetically edited human cells are isolated.

Another aspect described herein provides a population of genetically edited progenitor cells having at least one genetic modification resulting in decreased ELANE mRNA or protein expression.

Another aspect described herein provides a composition comprising any isolated genetically edited human cell or cell population described herein

Another aspect described herein provides a composition comprising any genetically edited progenitor cell or cell population described herein.

Another aspect described herein provides a method of treating a disease associated with abnormal ELANE gene expression, the method comprising, administering to a subject in need thereof a gene editing agent that targets the ELANE gene, wherein the edited ELANE gene comprises at least one mutation selected from the group consisting of a loss-of-function mutation, a through stop-gain mutation, a frameshift mutation, a premature termination codon, and a splicing mutation that encodes a premature termination codon.

In one embodiment of any aspect described herein, the disease is selected from the group consisting of chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis, chronic kidney disease, cyclic neutropenia, and severe congential neutropenia.

In one embodiment of any aspect described herein, the gene editing agent is selected from the group consisting of clustered regularly interspaced short palindromic repeats (CRISPR), Transcription activator-like effector nucleases (TALEN), Meganuclease, Zinc finger nucleases, Homologous recombination, and deaminase fusion proteins. In one embodiment of any aspect described herein, the gene editing agent is any modified synthetic nucleic acid, composition, or RNP complex described herein.

In one embodiment of any aspect described herein, the at least one mutation results in induction of nonsense-mediated decay, translational repression, or knockdown of transcript.

In one embodiment of any aspect described herein, the abnormal ELANE gene expression results in abnormal neutrophil elastase protein.

### Definitions

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given modified synthetic nucleic acid molecule, composition, or RNP complex described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. Where applicable, a decrease can be preferably down to a level accepted as within the range of normal for an individual without a given disorder.

As used herein, a “subject” means a human or animal. Usually the animal is a vertebrate such as a primate, rodent, domestic animal or game animal. Primates include, for example, chimpanzees, cynomologous monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of disease e.g., SCN. A subject can be male or female.

A subject can be one who has been previously diagnosed with or identified as suffering from or having a condition in need of treatment (e.g. a SCN) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

In one embodiment, the term “genetically edited” and its grammatical equivalents as used herein can refer to one or more human-designed alterations of a nucleic acid, e.g., the nucleic acid within an organism's genome. In another embodiment, genetically edited can refer to alterations, additions, and/or deletion of genes. A “genetically edited cell” can refer to a cell with an added, deleted and/or altered gene. The term “cell” or “genetically edited cell” and their grammatical equivalents as used herein can refer to a cell of human or non-human animal origin.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

As used herein, the term “DNA” is defined as deoxyribonucleic acid. The term “polynucleotide” is used herein interchangeably with “nucleic acid” to indicate a polymer of nucleosides. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e. the succession of letters used as abbreviations for bases) that biochemically characterizes a specific nucleic acid. A polynucleotide sequence presented herein is presented in a 5′ to 3′ direction unless otherwise indicated.

The term “ polypeptide ” as used herein refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a nonpolypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the term “administering,” refers to the placement of a therapeutic (e.g., a modified synthetic nucleic acid, composition or pharmaceutical composition, or RNP as disclosed herein) into a subject by a method or route which results in at least partial delivery of the therapeutic at a desired site. Pharmaceutical compositions comprising agents as disclosed herein can be administered by any appropriate route which results in an effective treatment in the subject.

A “nucleic acid”, as described herein, can be RNA or DNA, and can be single or double stranded, and can be selected, for example, from a group including: nucleic acid encoding a protein of interest, oligonucleotides, nucleic acid analogues, for example peptide-nucleic acid (PNA), pseudo-complementary PNA (pc-PNA), locked nucleic acid (LNA) etc. Such nucleic acid sequences include, for example, but are not limited to, nucleic acid sequence encoding proteins, for example that act as transcriptional repressors, antisense molecules, ribozymes, small inhibitory nucleic acid sequences, for example but are not limited to RNAi, shRNAi, siRNA, micro RNAi (mRNAi), antisense oligonucleotides etc.

The term “isolated cell” as used herein refers to a cell that has been removed from an organism in which it was originally found, or a descendant of such a cell. Optionally the cell has been cultured in vitro, e.g., in the presence of other cells. Optionally the cell is later introduced into a second organism or re-introduced into the organism from which it (or the cell from which it is descended) was isolated.

The term “isolated population” with respect to an isolated population of cells as used herein refers to a population of cells that has been removed and separated from a mixed or heterogeneous population of cells. In some embodiments, an isolated population is a substantially pure population of cells as compared to the heterogeneous population from which the cells were isolated or enriched. In some embodiments, the isolated population is an isolated population of human hematopoietic progenitor cells, e.g., a substantially pure population of human hematopoietic progenitor cells as compared to a heterogeneous population of cells comprising human hematopoietic progenitor cells and cells from which the human hematopoietic progenitor cells were derived.

The term “substantially pure,” with respect to a particular cell population, refers to a population of cells that is at least about 75%, preferably at least about 85%, more preferably at least about 90%, and most preferably at least about 95% pure, with respect to the cells making up a total cell population. That is, the terms “substantially pure” or “essentially purified,” with regard to a population of hematopoietic progenitor cells, refers to a population of cells that contain fewer than about 20%, more preferably fewer than about 15%, 10%, 8%, 7%, most preferably fewer than about 5%, 4%, 3%, 2%, 1%, or less than 1%, of cells that are not hematopoietic progenitor cells as defined by the terms herein.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean ±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the technology.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined within the description of the various aspects and embodiments of the technology of the following.

### BRIEF DESCRIPTION OF DRAWINGS

FIG. 1 shows the surface marker expression during neutrophil maturation.

FIG. 2 shows that severe congenital neutropenia is associated with a maturation block at or prior to the promyelocyte stage. Cell development for healthy individuals and SCN patients are shown. Definitions: CMP, common myeloid progenitor; GMP, granulocyte-macrophageprogenitor; MPP, multipotent progenitor; ST-HSC, short-term hematopoietic stem cell.

FIG. 3 shows that many ELANE mutations, mostly missense mutations, are associated with congenial neutropenia. Top shows severe congenital neutropenia phenotype and bottom shows cyclic neutropenia phenotype. Note there is some overlap with the same mutations associated with both phenotypes. Some SCN patients develop myelodysplastic syndrome (MDS) and/or acute myeloid leukemia (AML). The frameshift mutations are clustered at terminal exon 4 and exon 5. Totally 104 different ELANE mutations: 65 missense mutations; 15 frameshift; 8 termination; 8 intronic; 7 in-frame deletion or insertion; and 1 in 5′UTR.

FIG. 4 shows that frameshift mutations of ELANE result in −1 bp frameshift which leads to premature stop codon.

FIG. 5 shows that frameshift mutations of ELANE result in −1 bp frameshift which leads to premature stop codon. No frameshifts have been observed that result in −2 bp frameshift which would lead to a different aberrant protein. FIG. 5 presents SEQ ID NOs 375-380.

FIG. 6 presents a schematic of frameshift position and the consequence thereof. Frameshifts in late exons of ELANE that would cause SCN-like frameshifts and a neutrophil maturation arrest phenotype. In contrast frameshifts in early exons of ELANE would trigger nonsense mediated decay (NMD) and could rescue the phenotype caused by dominant acting SCN-associated mutations.

FIG. 7 shows that premature stop codons beyond 50 bp upstream of the last exon-exon junction evade NMD.

FIG. 8 shows that in vitro differentiation of neutrophils from CD34+ human hematopoietic stem and progenitor cells (HSPCs) results in mature neutrophil production (with CD11b+ CD16+ immunophenotype) that depends on dose of G-CSF. 3 ng/ml G-CSF is the minimum concentration to support efficient neutrophil maturation, so that is what was used in subsequent experiments.

FIG. 9 shows that on day 10 there is a range of cells from CD34+ HSPCs, CD34-CD117+CD16-promyelocytes, CD34-CD117-CD16-myelocytes, and CD34-CD117-CD16+ neutrophils (including bands and metamyelocytes).

FIG. 10 shows that schematic of guide RNA pooled screen to test all possible ELANE-targeting sgRNAs (chimeric single guide RNAs) as restricted by NGG protospacer adjacent motif (PAM) for SpCas9. HSPCs are infected with a lentiviral sgRNA library as well as with Cas9. Then cells are subject to in vitro neutrophil maturation conditions. Neutrophils and promyelocytes are sorted, genomic DNA isolated and guide RNA libraries prepared and sequenced to determine representation of guide RNAs in various cell types. The expected phenotype for guide RNAs that elicit a SCN-like phenotype would be relative enrichment in promyelocytes relative to neutrophils.

FIG. 11 shows the results of the sgRNA pooled screen show that sgRNAs targeting terminal portions of ELANE give an SCN-like phenotype with enrichment in promyelocytes relative to neutrophils. Many guides in exon 2 and exon 3 do not appear to elicit an SCN-like phenotype.

FIG. 12 shows the validation that a guide RNA targeting ELANE exon 5 causes a neutrophil maturation arrest phenotype with reduced neutrophils and increased promyelocytes. In contrast, a guide RNA targeting ELANE exon 2 avoids neutrophil maturation arrest.

FIG. 13 shows that the ELANE exon 5 targeting neutrophil maturation arrest phenotype may be partially rescued by increased concentration of G-CSF.

FIG. 14 shows that the editing at both exon 5 and exon 2 is highly efficient with almost no unedited alleles. For the exon 5 targeting, there is a shift from mainly −1 bp frameshifts in the promyelocytes to −2 bp and in-frame indels in the neutrophils. This indicates that the −1 bp frameshifts (which are the mutation type seen in SCN) are selectively associated with neutrophil maturation arrest. In contrast, there is no change in the pattern of editing between promyelocytes and neutrophils with exon 2 targeting, suggesting absence of selective pressure. Exon 5 frameshift—1 bp matches SCN associated mutation pattern. Exon 5 frameshift—2 bp has not been observed in SCN.

FIG. 15 shows that targeting indel mutations to ELANE exon 2 does no impact neutrophil maturation, whereas targeting ELANE exon 5 produces a SCN-like phenotype with neutrophil maturation arrest. Interestingly there is not only a relative increase in promyelocytes but also in HSPCs indicating the maturation block may sometimes occur earlier than the promyelocyte stage.

FIG. 16 shows that pairs of guide RNAs targeting exon 5 can selectively give −1 bp (top) or −2 bp (bottom) frameshifts respectively.

FIG. 17 demonstrates that inducing 1 bp frameshift in ELANE exon 5 produces stronger neutrophil maturation arrest phenotype as compared to 2 bp frameshift.

FIG. 18 demonstrates that inducing 1 bp frameshift in ELANE exon 5 produces stronger neutrophil maturation arrest phenotype as compared to 2 bp frameshift.

FIG. 19 shows a schematic inducing ELANE frameshifts at positions predicted to induce NMD results in preserved neutrophil maturation.

FIG. 20 demonstrates that three different guide sequences at ELANE exon 2 are shown to induce high efficiency of indels in CD34+ HSPCs (#2_1, #2_3, #2_4). Indel distribution as measured by Sanger sequence deconvolution. FIG. 20 presents SEQ ID NOs 381-384.

FIG. 21 demonstrates that pairs of guide RNAs result in precise targeted deletions if co-delivered. However, if delivery of individual guide RNAs is separated by at least 1 day the rate of precise targeted deletions is greatly reduced. Separating delivery of each guide RNA by 2 days abolishes large deletions.

FIG. 22 demonstrates that the sequential gene editing of ELANE to first generate SCN-like mutations (exon 5 1 bp frameshifts) and then test therapeutic rescue by targeting exon 2 for NMD induction. These results show that the neutrophil maturation block is rescued by exon 2 editing after exon 5 editing.

FIG. 23 demonstrates that the targeting of ELANE exon 2 induces nonsense mediated decay. ELANE mRNA was measured by RT-qPCR after editing of exon 2, exon 5, or exon 5 followed by exon 2.

FIG. 24 shows a cell proliferation curve of CD34+ HSPCs from a patient with a SCN (ELANE I118N) mutation. The figure demonstrates that CD34+ HSPCs from a patient with SCN (ELANE I118N) were edited at exon 2. Increased cell number was observed during neutrophil maturation.

FIGS. 25A and 25B demonstrate that CD34+ HSPCs from a patient with SCN (ELANE I118N) were edited at exon 2. (FIG. 25A) Loss of expression of ELANE mRNA were observed consistent with induction of NMD. (FIG. 25B) Editing ELANE exon 2 rescued neutrophil maturation arrest.

FIGS. 26A-26B show a schema of the CRISPR screen nominates potential therapeutic and disease modeling guide RNAs for severe congenital neutropenia targeting ELANE. (FIG. 26A) Scheme of CRISPR/Cas9 screening in human hematopoietic stem and progenitor cells (HSPCs). After 2-day recovery in vitro, human HSPCs cells were transduced with Sendai virus expressing SpCas9 and lentivirus expressing a library of 273 guide RNAs targeting the ELANE coding sequences. G-CSF were used to drive neutrophil lineage differentiation in vitro for 10 days, followed by sorting of both CD34-CD117-CD16+ (bands/neutrophils) and CD34-CD117+CD16-(promyelocytes/myelocytes) and subsequent deep sequencing to determine the abundance of each guide RNA. N =3 replicates. (FIG. 26B) Results of the pooled guide RNA CRISPR screen showing promyelocyte-to-neutrophil enrichment score as log2 fold change (L2FC). Guide RNAs targeting late exons of ELANE, in particular those at positions predicted to escape nonsense mediated decay (downstream from 55 nt upstream of final exon-exon boundary), tend to have positive promyelocyte-to-neutrophil enrichment scores, suggesting neutrophil maturation arrest. In contrast, guide RNAs targeting early exons of ELANE tend to have more neutral enrichment scores, suggesting intact neutrophil maturation.

FIG. 27 shows a May-Grünwald staining of HSPCs-derived in vitro cultures. In vitro neutrophil maturation recapitulates normal neutrophil development. After 10-day in vitro differentiation towards neutrophils, cells were sorted into CD34-CD117+CD16-, CD34-CD117-CD16- and CD34-CD117-CD16+ cell populations. May-Grünwald staining were performed to deterimine to the cell types in each sorted cell population, where CD34-CD117+CD16-group was composed of mostly promyeocytes, CD34-CD117-CD16- mostly promyelocytes and myelocytes, and CD34-CD117-CD16+ predominantly bands

FIGS. 28A-28E show that targeting ELANE early exons leads to nonsense mediated decay and overcomes neutrophil maturation arrest. Depicted are guides targeting 5 to mimic neutrophenia phenotype, while early exon targeting for therapy. FIGS. 28A and 28B show human HSPCs cells that were edited by RNP electroporation with guide RNAs targeting ELANE exon 2, ELANE exon 5 and a control guide RNA targeting a neutral locus separately. Expansion of different cell populations were calculated by total cell expansion fold (panel B, day 10 relative to day 1) and percentage of each population determined by flow cytometry at day 10 (N>=3). Indels were measured by Sanger sequencing with deconvolution. ELANE mRNA level was determined by real-time PCR at day 5 (panel A, totally 10-day neutrophil differentiation process). FIGS. 28C-28E show human HSPCs that were sequentially edited (48 hours between rounds of electroporation) with two different RNPs. (FIG. 28C) “E5-3+E2-3” indicates simultaneous delivery of both guide RNAs which results in interstitial deletion between the two genomic target sites. In contrast final four lanes labeled “E5-3, E2-3”, “E5-3, neutral” etc. indicate sequential delivery, with for example first delivery of RNP E5-3 targeting ELANE exon 5 and later delivery of RNP E2-3 targeting ELANE exon 2. “Neutral” indicates a neutral genomic locus. Sequential delivery avoids interstitial deletion. PCR was performed to amplify a genomic region of 3579 bp covering both exon 2 and exon 5. When the two guide RNAs ELANE E2-3 (exon 2) and E5-3 (exon 5) were present in the cells simultaneously, there was a deletion of long fragment between E2-3 and E5-3, leading to a shortened PCR product of 559 bp. PCR product of neutral locus was used as loading control. (FIG. 28D) Cell expansion as determined by cell counting and flow cytometry. Indels by Sanger sequencing. E. ELANE mRNA expression by RT-qPCR. These results show that targeting ELANE exon 5 (E5-3 guide RNA) produces a neutrophil maturation arrest phenotype, but that targeting ELANE exon 2 (guide RNA E2-3) produces ELANE transcript decay and overcomes neutrophil maturation arrest. N =3 replicates.

FIG. 29 shows that sequential gene editing enables disease modeling edits followed by therapeutic edits. The figure depicts that Sequential delivery enables double ELANE editing without long deletion. Upper panel: Scheme of locations of guide RNAs targeting ELANE exon 2 and exon 5, as well as primers for PCR. Middle panel: Expected sequence of PCR product with long-fragment deletion if both guide RNAs (Exon E2-3 and Exon 5-3) were present within the cells simultaneuously (SEQ ID NO: 386). Lower panel: Human HSPCs cells were edited by two guide RNAs targeting either ELANE exon 2, ELANE exon 5 or a control guide RNA targeting a neutral locus in 0-day, 1-day, 2-day, 3-day or 4-day interval. Human HSPCs cells which were edited by Exon E5-3, Exon E2-3 and neutral locus guide were used as controls. Genomic DNA was extracted at day 6, followed by PCR.

FIG. 30 shows increased Annexin V+ in promyelocytes/myelocytes due to ELANE exon 5 editing. Disease modeling edits cause neutrophil precursor cell death, while therapeutic edits avoid neutrophil precursor cell death. Human HSPCs cells were edited by guide RNAs targeting ELANE exon 2, ELANE exon 5 and a control guide RNA targeting a neutral locus separately. Annexin V staining were performed from day 2 to day 11 during a total of 11-day neutrophil differentiation course. Percentage of Annexin V+ cells in CD34+, CD34-CD117+CD16-, CD34-CD117-CD16− and CD34-CD117-CD16+ were calculated separately (N=3 replicates).

FIG. 31 shows that therapeutic gene editing of primary ELANE mutated severe congenital neutropenia (SCN) human cells overcomes disease pathobiology. CD34+ HSPCs were isolated from the bone marrow of SCN patient 1 with ELANE P139L mutation. Cells were electroporated with SpCas9:sgRNA RNP with gRNA e2-3 targeting ELANE early exon (or a control RNP targeting a functionally neutral locus). At the end of in vitro differentiation, cells were prepared as a cytospin to evaluate morphology. Neutrophils were counted by a hematopathologist blinded to sample identity. Neutrophil number was compared to the starting HSPC number.

FIG. 32 shows that therapeutic gene editing of primary ELANE mutated severe congenital neutropenia (SCN) patient cells overcomes disease pathobiology. CD34+ HSPCs were isolated from the bone marrow of ELANE mutated SCN patient 1 with P139L, patient 2 with I118N, patient 3 with L84P, and patient 4 with M1del mutations. Cells were electroporated with SpCas9:sgRNA RNP with gRNA e2-3 targeting ELANE early exon (or a control RNP targeting a functionally neutral locus). At the end of in vitro differentiation, cells were prepared as a cytospin to evaluate morphology. Neutrophils were counted by a hematopathologist blinded to sample identity. Neutrophil number was compared to the starting HSPC number.

FIG. 33 shows a marker panel used for mice bone marrow immunophenotyping. Human xenograft to mice recapitulates normal hematopoiesis including normal neutrophil development. Gene edited healthy donor human CD34+ HSPCs were electroporated with SpCas9:sgRNA RNP, then 24 hours later infused intravenously to immunodeficient mice (NBSGW strain mice). Immunophenotyping marker panel of NBSGW mice bone marrow 16 weeks after xeno-engraftment of edited human HSPCs is shown. Five populations were sorted for May-Grünwald staining, including hCD45+SSChighCD33lowCD16+ (exclusively bands/neutrophils), hCD45+SSChighCD33lowCD16dim (mostly segmented), hCD45+SSChighCD33lowCD16− (neutrophil precursors), hCD45+SSClowCD33highCD14+ (monocytes) and hCD45+SSClowCD33highCD14− (myeloid precursors).

FIG. 34 shows the myelopoiesis arrest in NSBGW mice due to ELANE exon 5, not exon 2, targeting. Therapeutic gene editing of ELANE in human hematopoietic stem cells preserves normal hematopoietic functions including neutrophil maturation. Cell population distribution of NBSGW mice BM (16 weeks after xeno-engraftment of edited human HSPCs). The disease modeling guide RNA (E5-3 targeting ELANE exon 5) produced neutrophil maturation arrest. In contrast the therapeutic class candidate guide RNA (E2-3 targeting ELANE exon 2) did not impair neutrophil development nor did it impact overall hematopoietic stem cell self-renewal or multilineage differentiation.

FIG. 35 shows the myelopoiesis arrest in NSBGW mice due to ELANE exon 5, not exon 2, targeting. Therapeutic gene editing of ELANE in human hematopoietic stem cells preserves normal hematopoietic functions including neutrophil maturation. Cell population distribution of NBSGW mice BM (16 weeks after xeno-engraftment of edited human HSPCs). The disease modeling guide RNA (E5-3 targeting ELANE exon 5) produced neutrophil maturation arrest. In contrast the therapeutic class candidate guide RNA (E2-3 targeting ELANE exon 2) did not impair neutrophil development nor did it impact overall hematopoietic stem cell self-renewal or multilineage differentiation.

FIG. 36 shows the myelopoiesis arrest in NSBGW mice due to ELANE exon 5, not exon 2, targeting. Therapeutic gene edits persist at high levels through all hematopoietic lineages. Different human cell populations were sorted from mice BM, and the indel allele frequency were analyzed in each population. ELANE early exon targeting guide RNAs (therapeutic edits) efficiently produce indels in the input cell graft, and these indels persisted at high levels in all hematopoietic lineages including in neutrophils 16 weeks after xenotransplantation. High editing efficiency was also observed in the ELANE late exon gene edited input cell product (disease modeling edits). These indels were preserved at high levels in all hematopoietic lineages except the neutrophil series. Indels were especially diminished in mature neutrophils, consistent with neutrophil maturation arrest of these disease modeling gene edited cells.

FIG. 37 shows the gene editing of ELANE exon 5 with −1 frame indels selectively models SCN in vivo. Therapeutic gene edits persist at high levels through all hematopoietic lineages. Most of the the therapeutic gene edits in the input graft, and in the engrafting BM HSPCs were comprised of −2 frame indels (ELANE exon 2 E2-3). These indels persisted at high levels across all hematopoietic lineages, including neutrophils, suggesting these edits have no negative impact on neutrophil development. In contrast, most of the the disease modeling gene edits in the input graft, and in the engrafting BM HSPCs were comprised of −1 frame indels (ELANE exon 5 E5-3). The surviving neutrophils after late exon editing showed depletion of −1 frame indels consistent with the failure of cells bearing these mutations to mature. As expected, unedited alleles were relatively enriched in the surviving neutrophils. Surprisingly, −2 frame indels were also enriched in surviving neutrophils.

FIG. 38 shows that ELANE frame −1 bp, not −2 bp, results in myelopoiesis arrest. Disease modeling ELANE late exon gene edits show that −1 frame indels produce neutrophil maturation arrest; in contrast −2 frame indels are compatible with intact neutrophil maturation. In order to further examine whether ELANE late exon frame −1 or −2 indels caused neutrophil maturation arrest, multiple guide RNAs targeting late exon 4 or exon 5, frameshifts expected to escape nonsense mediated decay, were tested. Indels were determined by amplicon sequencing 5 days after RNP electroporation, cell counting was performed at day 0 and day 10 (10-day differentiation period) and flow cytometry was done at day 10. Cell expansion fold was calculated by percentage of cell populations from flow cytometry and total cell expansion fold at day 10 relative to day 0. Pearson correlation was carried out between percentage of frame −1 and expansion fold of different populations.

FIG. 39 shows a correlation between different types of indels and neutrophil percentage in vitro. Disease modeling ELANE late exon gene edits show that −1 frame indels produce neutrophil maturation arrest; in contrast −2 frame indels are compatible with intact neutrophil maturation. In order to further examine whether ELANE late exon frame −1 or −2 indels caused neutrophil maturation arrest, multiple guide RNAs targeting late exon 4 or exon 5, frameshifts expected to escape nonsense mediated decay, were tested. Indels were determined by amplicon sequencing 5 days after RNP electroporation, cell counting was performed at day 0 and day 10 (10-day differentiation period) and flow cytometry was done at day 10. Cell expansion fold was calculated by percentage of cell populations from flow cytometry and total cell expansion fold at day 10 relative to day 0. Pearson correlation was carried out between percentage of frame −1 and expansion fold of different populations.

FIG. 40 shows the disease modeling of the ELANE late exon gene edits. It shows show that −1 frame indels produce neutrophil maturation arrest; in contrast −2 frame indels are compatible with intact neutrophil maturation. In order to further examine whether ELANE late exon frame −1 or −2 indels caused neutrophil maturation arrest, multiple guide RNAs targeting late exon 4 or exon 5, frameshifts expected to escape nonsense mediated decay, were tested. Indels were determined by amplicon sequencing 5 days after RNP electroporation, cell counting was performed at day 0 and day 10 (10-day differentiation period) and flow cytometry was done at day 10. Cell expansion fold was calculated by percentage of cell populations from flow cytometry and total cell expansion fold at day 10 relative to day 0. Pearson correlation was carried out between percentage of frame −1 and expansion fold of different populations.

FIG. 41 shows that naturally occurring ELANE late exon gene edits are −1 frame indels, not −2 frame indels, are associated with severe congenital neutropenia. Congenital neutropenia genotype data were analyzed.

FIG. 42 shows naturally occurring ELANE late exon gene edits. Pooled CRISPR screen show that −1 frame indels from AA 180-238, not −2 frame indels or later −1 frame indels, are associated with severe congenital neutropenia. CRISPR dense mutagenesis provides clues into disease mechanisms and theraputic potential. ELANE late exon frameshift mutations could be classified based on the nature of the termination codon encoded (shown with asterisk). For −1 frame, depending on position of frameshift, 4 distinct termination codons are possible (4 patterns). Pattern 1-3 are associated with congenital neutropenia. Likewise guide RNAs predicted to model pattern 1-3 were associated with a neutrophil maturation arrest phenotype, but not guide RNAs modeling pattern 4.

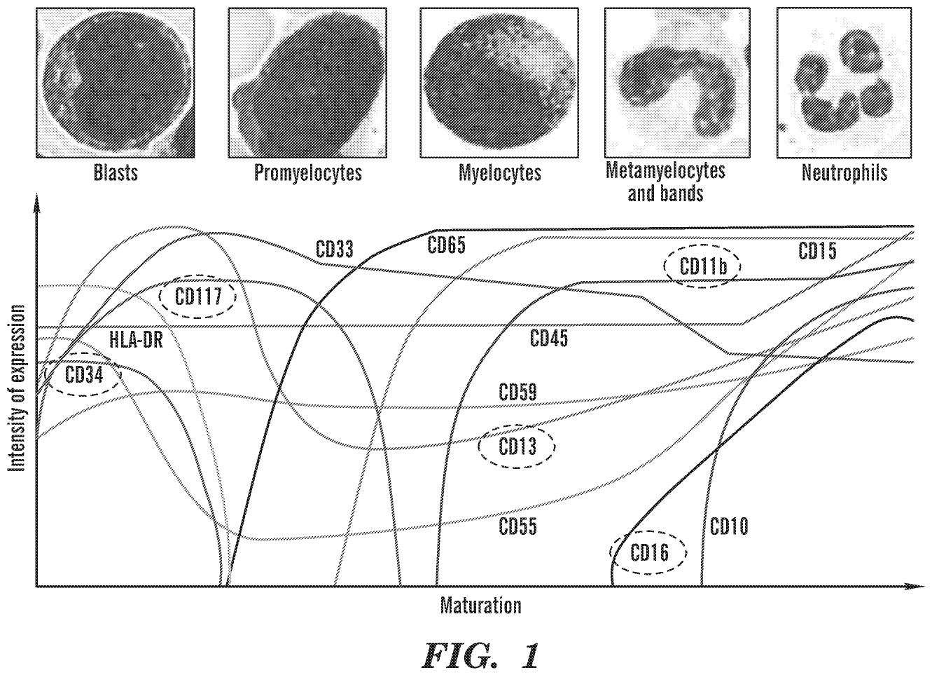

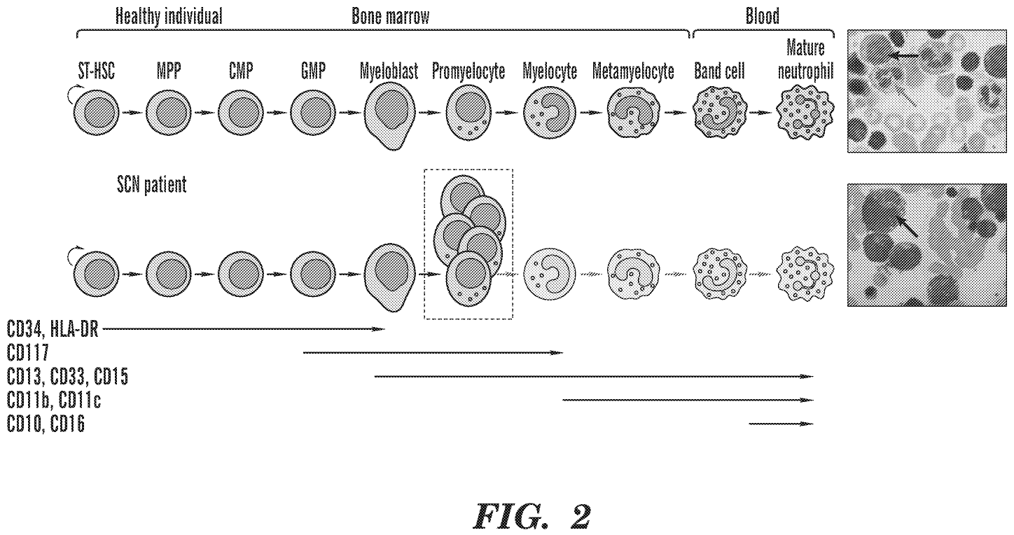

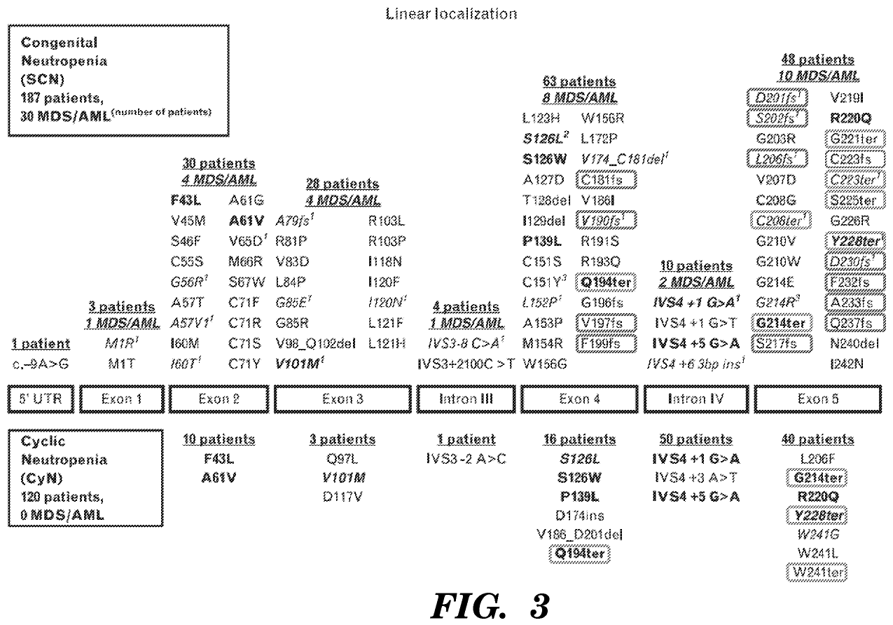

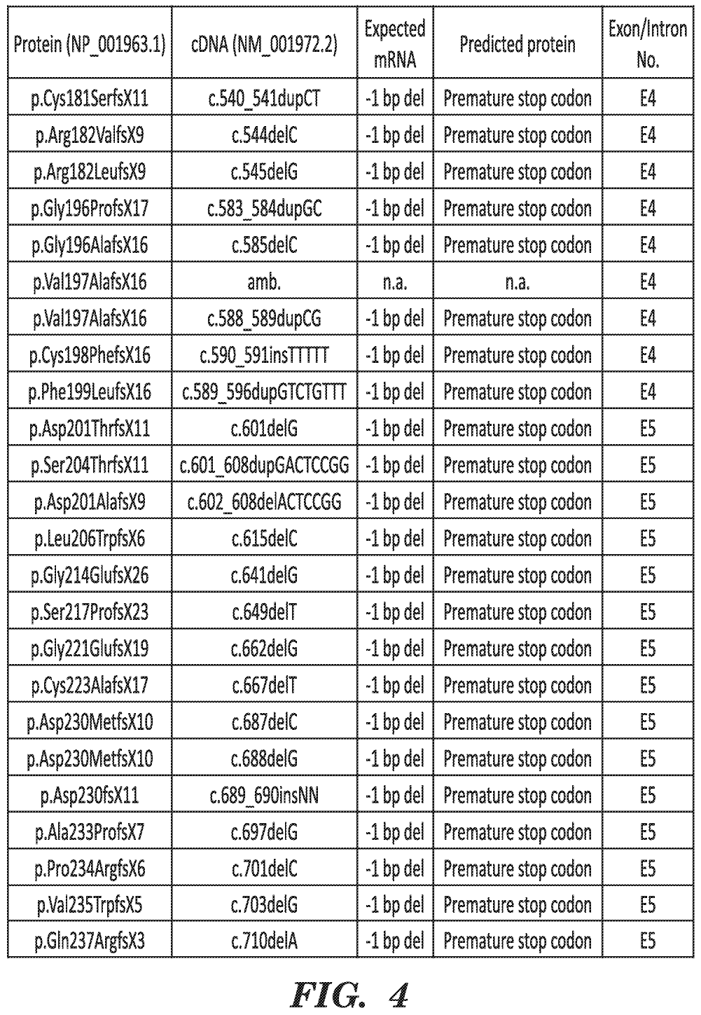

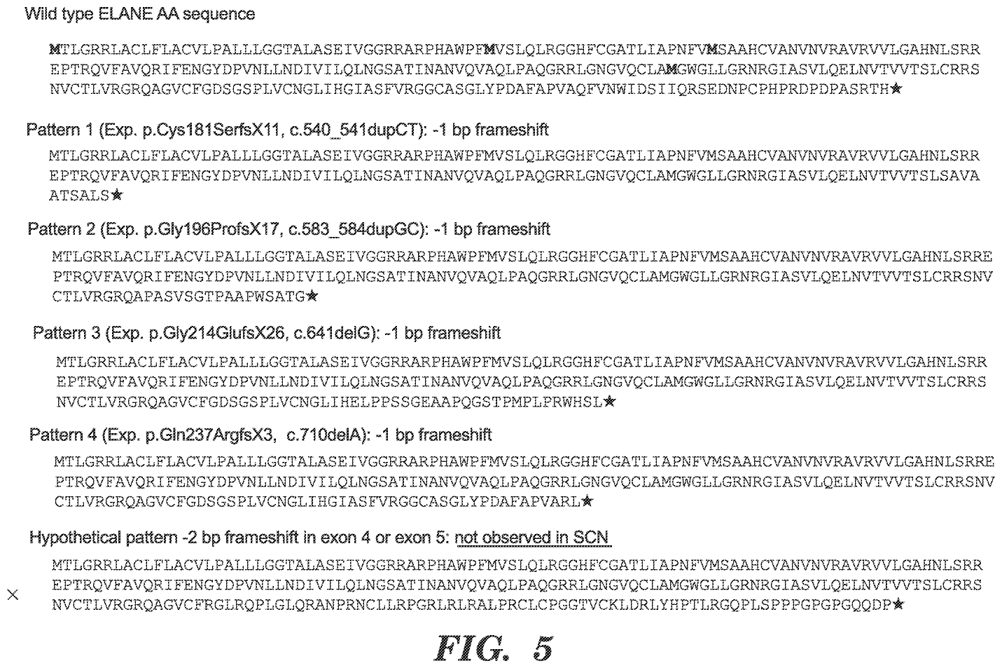

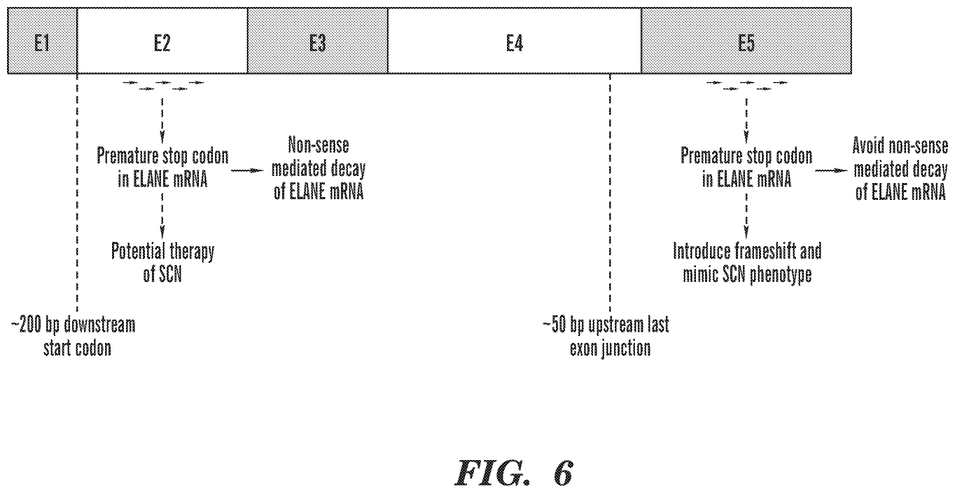

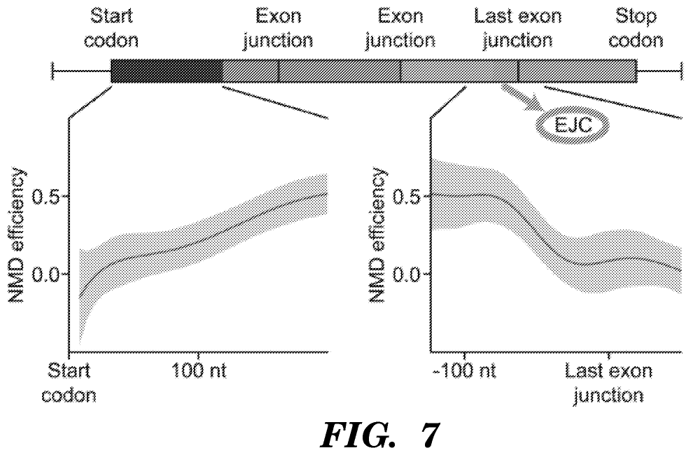

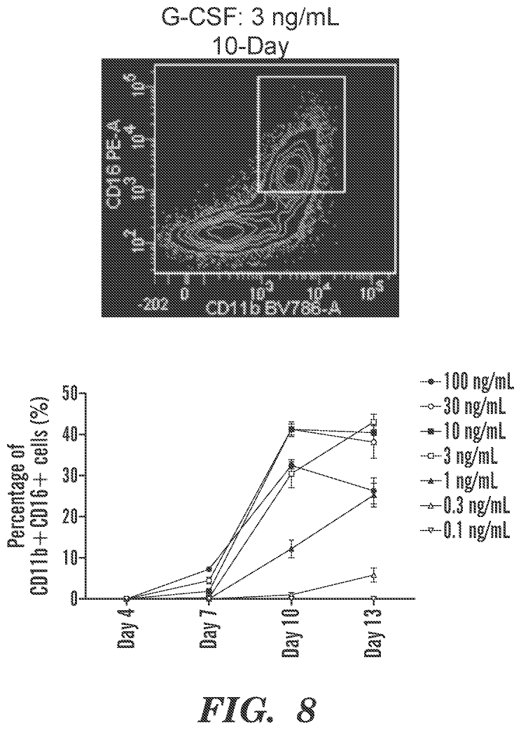

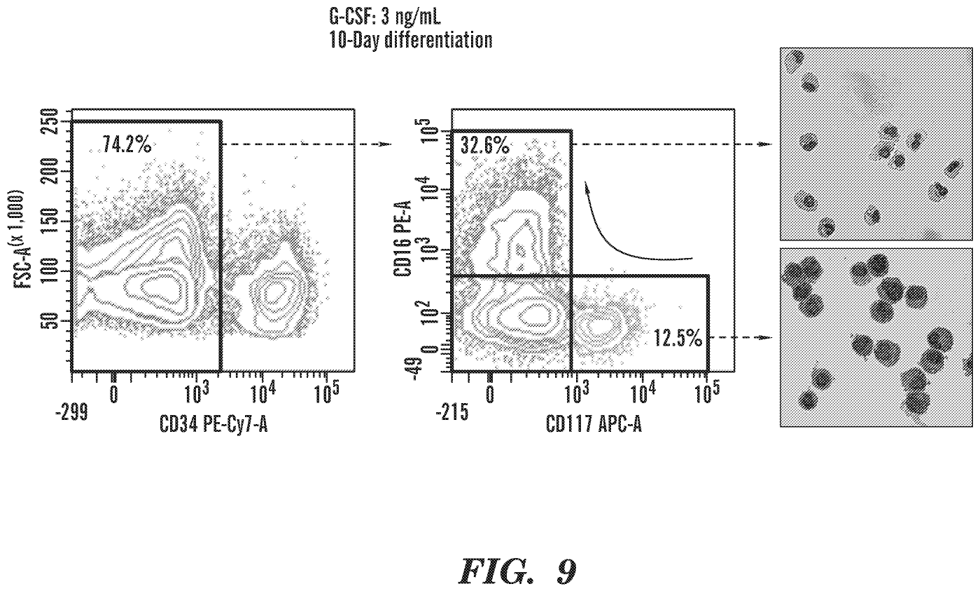

### DETAILED DESCRIPTION

Reagents and methods described herein are designed for the inhibition of the ELANE gene. The ELANE gene is required for the production of neutrophil elastase, a protein found in neutrophils. When an immune response is initiated in response to an infection, neutrophils release neutrophil elastase as a means to modifies the function of certain cells and proteins to fight the infection. Elastases form a subfamily of serine proteases that hydrolyze many proteins in addition to elastin. Humans have six elastase genes which encode structurally similar proteins. The encoded preproprotein is proteolytically processed to generate the active protease. Following activation, this protease hydrolyzes proteins within specialized neutrophil lysosomes, called azurophil granules, as well as proteins of the extracellular matrix. The enzyme may play a role in degenerative and inflammatory diseases through proteolysis of collagen-IV and elastin. This protein also degrades the outer membrane protein A (OmpA) of E. coli as well as the virulence factors of such bacteria as Shigella, Salmonella and Yersinia.

Mutations in the ELANE gene have been found to result in Cyclic Neutropenia and Severe Congenital Neutropenia, disease associated with abnormal neutrophil elastase formation and neutrophil death, and a neutrophil deficiency. Frame shift mutation identified in the ELANE gene have been shown to result in stable neutrophil elastase, which fails to undergo normal degradation. This stable form of neutrophil elastase accumulates in the neutrophils, damaging or killing the neutrophil. Data presented herein show that inhibition of the stable ELANE gene via CRISPR gene editing system reverses the disease state.

Abnormal neutropil elastase also contributes to the pathology of a variety of human diseases, including inflammatory and degenerative conditions such as chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis and chronic kidney disease⁴⁻⁹. Methods and compositions as described herein to inhibit the levels and/or activity of the ELANE can be considered a therapeutic approach for all these inflammatory conditions in which abnormal neutrophil elastase contributes to pathology.

Methods and compositions described herein require that the levels and/or activity of the ELANE gene are inhibited. As used herein, “Elastase, Neutrophil Expressed,” or “ELANE,” also known as GE, NE, HLE, HNE, ELA2, SCN1, and PMN-E refers to a gene that encodes the neutrophil elastase protein. ELANE sequences are known for a number of species, e.g., human ELANE (NCBI Gene ID: 1991) polypeptide (e.g., NCBI Ref Seq NP_001963.1) and mRNA (e.g., NCBI Ref Seq NM_001972.4). ELANE can refer to human ELANE, including naturally occurring variants, molecules, and alleles thereof. ELANE refers to the mammalian ELANE of, e.g., mouse, rat, rabbit, dog, cat, cow, horse, pig, and the like. The nucleic sequence of SEQ ID NO: 385 comprises a nucleic sequence which encodes ELANE.

| (SEQ ID NO: 385) | |
|---|---|
| | cccaccatga ccctcggccg ccgactcgcg tgtcttttcc |
| 61 | tcgcctgtgt cctgccggcc ttgctgctgg ggggcaccgc gctggcctcg gagattgtgg |
| 121 | ggggccggcg agcgcggccc cacgcgtggc ccttcatggt gtccctgcag ctgcgcggag |
| 181 | gccacttctg cggcgccacc ctgattgcgc ccaacttcgt catgtcggcc gcgcactgcg |
| 241 | tggcgaatgt aaacgtccgc gcggtgcggg tggtcctggg agcccataac ctctcgcggc |
| 301 | gggagcccac ccggcaggtg ttcgccgtgc agcgcatctt cgaaaacggc tacgaccccg |
| 361 | taaacttgct caacgacatc gtgattctcc agctcaacgg gtcggccacc atcaacgcca |
| 421 | acgtgcaggt ggcccagctg ccggctcagg gacgccgcct gggcaacggg gtgcagtgcc |
| 481 | tggccatggg ctggggcctt ctgggcagga accgtgggat cgccagcgtc ctgcaggagc |
| 541 | tcaacgtgac ggtggtgacg tccctctgcc gtcgcagcaa cgtctgcact ctcgtgaggg |
| 601 | gccggcaggc cggcgtctgt ttcggggact ccggcagccc cttggtctgc aacgggctaa |
| 661 | tccacggaat tgcctccttc gtccggggag gctgcgcctc agggctctac cccgatgcct |
| 721 | ttgccccggt ggcacagttt gtaaactgga tcgactctat catccaacgc tccgaggaca |
| 781 | acccctgtcc ccacccccgg gacccggacc cggccagcag gacccactga |

The methods and compositions described herein relate, in part, to the discovery that in vitro for Cas9:sgRNA ribonucleoprotein (RNP) electroporation of progenitor cells in a solution comprising glycerol is an effective method for producing viable edited progenitor cells that have (i) high on-target indel frequency (the result of targeted editing directed by the specific modified synthetic sgRNA), (ii) no detectable off-target editing, (iii) reduced ELANE mRNA or protein expression, and (v) lack detectable genotoxicity. Furthermore, this electroporation approach eliminates the need for selection of the resultant cells after CRISPR-base gene editing. Importantly, Electroporation in the presence of glycerol increases the viability of cells now comprising RNP.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In some embodiments, a target sequence is located in the nucleus or cytoplasm of a cell. In some embodiments, the target sequence may be within an organelle of a eukaryotic cell, for example, mitochondrion or chloroplast. A sequence or template that may be used for recombination into the targeted locus comprising the target sequences is referred to as an “editing template” or “editing polynucleotide” or “editing sequence”. In aspects of the invention, an exogenous template polynucleotide may be referred to as an editing template. In an aspect of the invention the recombination is homologous recombination.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of a CRISPR system are introduced into a cell such that expression of the elements of the CRISPR system direct formation of a CRISPR complex at one or more target sites. For example, an NLS-Cas fusion enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

### Modified Synthetic Nucleic Acid and Ribonucleoproteins (RNP)

One aspect described herein provides a modified synthetic nucleic acid having a sequence comprising, consisting of, or consisting essentially of SEQ ID NOs 1-374, or as listed in Table 1, wherein there is at least one chemical modification to a nucleotide in the nucleic acid molecule.

In one embodiment, the modified synthetic nucleic acid that targets and hybridizes to a target sequence of a DNA molecule. As used herein, “hybridizes” or “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

In one embodiment, the at least one modification is selected from 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′- terminal cap known from nature, an xRNA base (analogous to “xDNA” base), an yRNA base (analogous to “yDNA” base), a PEG substituent, and a conjugated linker to a dye or non-fluorescent label (or tag).

In one embodiment, a chemical modification is located at one or more terminal nucleotides in the nucleic acid molecule. The location of a chemical modification is not limiting and can be found at any nucleotide along the sequence of a modified synthetic nucleic acid. For example, a chemical modification can be located only at the 3′ end, or only at the 5′ end, or at both the 5′ and 3′ ends of the synthetic nucleic acid molecule, or the chemical modification is located to first three nucleotides and to the last three nucleotides of the synthetic nucleic acid molecule.

In one embodiment, the modified synthetic molecule further comprises a crRNA/tracrRNA sequence.

In one embodiment, the modified synthetic nucleic acid described herein is a single guide RNAs. In general, a guide RNA sequence is any polynucleotide sequence having sufficient complementarity with a target polynucleotide sequence to hybridize with the target sequence (e.g., ELANE) and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In one embodiment, the ELANE gene is edited to produce loss-of-function, through stop-gain, or frameshift mutation, or induction of nonsense-mediated decay, or translational repression, or knockdown of transcript are used. In one embodiment, the ELANE gene is edited to encode a premature termination codon. In another embodiment, the ELANE gene is edited to alter splicing, such that a premature termination codon is encoded.

In one embodment, the ELANE gene is edited using a gene editing system other than CRISPR. Non-limiting examples of encompassed gene editing methods include, e.g., zinc finger nucleases (ZFNs), transcription activator-like effector nucleases (TALENs), Homologous recombination, meganucleases, and base-editors.

Meganuclease may be, but is not limited to, a naturally-occurring meganuclease, which recognizes 15 to 40 base pair cleavage sites, which are usually classified into four families: LAGLIDADG family, the GIY-YIG family, His-Cyst box family, and HNH family. The exemplary meganuclease includes I-Scel, I-Ceul, PI-PspI, PI-SceI, I-SceIV, I-CsmI, I-PanI, I-SceII, I-PpoI, I-SceIII, I-Crel, I-TevI, I-TevII, and I-TevIII.

In some embodiments, the ELANE gene is edited using a deaminase fusion protein. In some embodiments, the deaminase includes, but is not limited, to a cytosine deaminase-based editor (e.g. the target base is a cytosine (C) base and the deamination of the target C base results in a C to thymine (T) change to produce loss-of-function alleles through stop-gain or splice site mutations. In some embodiments, the deaminase includes but is not limited to an adenosine deaminase-based base editor (e.g. the target base is an adenosine (A) base and the deamination of the target A base results in A to guanine (G) change to produce loss-of-function alleles through stop-gain or splice site mutations.

In one embodiment, programmable nucleases are used to edit the ELANE gene. The term “programmable nuclease” used in the present disclosure refers to all forms of nuclease that is capable of recognizing and cleaving a specific site on a desired genome. In particular, it may include, but is not limited to, a transcription activator-like effector nuclease (TALEN) fused with a transcription activator-like effector (TAL) domain derived from a plant pathogenic gene, which is a domain recognizing a specific target sequence on a genome, and a cleavage domain, zinc-finger nuclease, meganuclease, RGEN (RNA-guided engineered nuclease) derived from CRISPR, which is a microbial immune system, Cpf1, Ago homolog (DNA-guided endonuclease), etc. The programmable nucleases recognize specific base sequences in the genome of animal and plant cells, including human cells, to cause double strand breaks (DSBs). The double strand breaks include both the blunt end or the cohesive end by cleaving the double strands of DNA. DSBs are efficiently repaired by homologous recombination or non-homologous end-joining (NHEJ) mechanisms within the cell, which allows researchers to introduce desired mutations into on-target sites during this process. The programmable nucleases may be artificial or manipulated non-naturally occurring. As used herein, the term “on-target site” relates to a site to which a mutation is to be introduced by using programmable nucleases, and may be selected arbitrarily depending on the purpose thereof It may be a non-coding DNA sequence that can be present within a specific gene and does not produce a protein. The programmable nucleases have sequence specificity, and thus work at an on-target site, but may work at an off-target site depending on the target sequence. As used herein, the term “off-target site” relates to a site where the programmable nucleases have activity at a site having a sequence that is not identical to the target sequence of the programmable nucleases. That is, it refers to a site other than an on-target site that is cleaved by the programmable nucleases. In particular, the off-target site in the present disclosure includes not only the actual off-target site for a specific programmable nuclease but also the site where it is likely to become an off-target site. The off-target site may be, but is not limited to, a site cleaved by programmable nucleases in vitro.

In one embodiment, any modified synthetic nucleic acid sequence described herein is used in combination with a DNA-targeting endonuclease Cas protein. Non-limiting examples of Cas proteins include Cpf1, C2c1, C2c3, Cas12a, Cas12b, Cas12c, Cas12d, Cas12e, Cas13a, Cas13b, and Cas13c. Cas1, Cas1B, Cas2, Cas3, Cas4, Cas5, Cas6, Cas7, Cas8, Cas9 (also known as Csn1 and Csx12), Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, and Cas13c homologs thereof, or modified versions thereof. These enzymes are known; for example, the amino acid sequence of S. pyogenes Cas9 protein may be found in the SwissProt database under accession number Q99ZW2. In some embodiments, the Cas protein comprises DNA cleavage activity, such as Cas9. In some embodiments the Cas protein is Cas9, and may be Cas9 from S. pyogenes (for example spCas9 or variant thereof), from Staphylococcus aureus (SaCas9) or from S. pneumoniae. In some embodiments, the Cas9 Cas9 enzyme is from, or is derived from, spCas9 or saCas9. By derived, Applicants mean that the derived enzyme is largely based, in the sense of having a high degree of sequence homology with, a wildtype enzyme, but that it has been mutated (modified) in some way as described herein.

In some embodiments, the Cas protein directs cleavage of one or both strands at the location of a target sequence, such as within the target sequence and/or within the complement of the target sequence. In some embodiments, the Cas protein directs cleavage of one or both strands within about 1, 2, 3, 4, 5, 6, 7, 8, 9, 10, 15, 20, 25, 50, 100, 200, 500, or more base pairs from the first or last nucleotide of a target sequence.

In one embodiment, the Cas protein comprising at least one nuclear localization signal sequences (NLSs). In some embodiments, the Cas protein comprises at least one NLSs at or near the amino-terminus, at least one NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. Typically, an NLS consists of one or more short sequences of positively charged lysines or arginines exposed on the protein surface, but other types of NLS are known. Non-limiting examples of NLSs include an NLS sequence derived from: the NLS of the SV40 virus large T-antigen, having the amino acid sequence PKKKRKV (SEQ ID NO: 387); the NLS from nucleoplasmin (e.g. the nucleoplasmin bipartite NLS with the sequence KRPAATKKAGQAKKKK (SEQ ID NO: 388)); the c-myc NLS having the amino acid sequence PAAKRVKLD (SEQ ID NO: 389) or RQRRNELKRSP (SEQ ID NO: 390); the hRNPA1 M9 NLS having the sequence NQSSNFGPMKGGNFGGRSSGPYGGGGQYFAKPRNQGGY (SEQ ID NO: 391); the sequence RMRIZFKNKGKDTAELRRRRVEVSVELRKAKKDEQILKRRNV (SEQ ID NO: 392) of the IBB domain from importin-alpha; the sequences VSRKRPRP (SEQ ID NO: 393) and PPKKARED (SEQ ID NO: 394) of the myoma T protein; the sequence PQPKKKPL (SEQ ID NO: 395) of human p53; the sequence SALIKKKKKMAP (SEQ ID NO: 396) of mouse c-abl IV; the sequences DRLRR (SEQ ID NO: 397) and PKQKKRK (SEQ ID NO: 398) of the influenza virus NS1; the sequence RKLKKKIKKL (SEQ ID NO: 399) of the Hepatitis virus delta antigen; the sequence REKKKFLKRR (SEQ ID NO: 400) of the mouse Mx1 protein; the sequence KRKGDEVDGVDEVAKKKSKK (SEQ ID NO: 401) of the human poly(ADP-ribose) polymerase; the sequence RKCLQAGMNLEARKTKK (SEQ ID NO: 402) of the steroid hormone receptors (human) glucocorticoid; the sequence GKRKLITSEEERSPAKRGRKS (SEQ ID NO: 403) of 53BP1; the sequence KRKRRP (SEQ ID NO. 404) of BRCA1; the sequence KRKGSPCDTLASSTEKRRRE (SEQ ID NO. 405) of SRC-1; and the sequence KRNFRSALNRKE (SEQ ID NO: 406) of IRF3.

In one embodiment, the chemical modification is only found on the 5′ end of the modified synthetic nucleic acid molecule. In another embodiment, the chemical modification is found only on the 3′-end. Methods of chemical modification are known in the art. For example, as described in Hendel et al., 2015, Nature Biotechnology, the reference is incorporated herein in its entirety. Chemically modified guide RNAs are commercially available and can purchased, e.g., from Synthego.

In one embodiment of this aspect and all other aspects described herein, the chemical modification is selected from the group consisting of 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′-F-arabin, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag). Chemical modifications of gRNA are further described in, e.g., Hendel A, et al. Nat Biotechnol. 2015 September; 33(9): 985-989, which is incorporated herein by reference in its entirety. Methods for generating chemical modifications on gRNA are further reviewed in, e.g., International Patent Application WO2016/089433, which is incorporated herein by reference in its entirety.

In one embodiment of this aspect and all other aspects described herein, the chemical modification is located only at the 3′ end, or added only at the 5′ end, or added at both the 5′ and 3′ ends of the modified synthetic nucleic acid molecule.

In one embodiment of any one aspects described herein, the chemical modification is located to first three nucleotides and to the last three nucleotides of the modified synthetic nucleic acid molecule.

In one embodiment of any one aspects described herein, the nucleic acid sequence further comprising a crRNA/tracrRNA sequence. The crRNA/tracrRNA sequence is a hybrid sequence for the binding of DNA-targeting endonuclease is a Cas (CRISPR-associated) protein in forming the gene editing ribonucleoprotein (RNP) complex. In one embodiment, the tracr sequence may comprise or consist of all or a portion of a wild-type tracr sequence (e.g. about or more than about 20, 26, 32, 45, 48, 54, 63, 67, 85, or more nucleotides of a wild-type tracr sequence), and may also form part of a CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

One aspect provides a ribonucleoprotein (RNP) complex comprising any of the modified synthetic nucleic acids described herein and a DNA-targeting endonuclease Cas protein.

In one embodiment, the RNP complex is for use in an ex vivo method of producing a progenitor cell or a population of progenitor cell wherein the cells or the differentiated progeny thereof have decreased ELANE mRNA or protein expression.

In another embodiment, the RNP complex is for use in an ex vivo method of producing an isolated genetic engineered human cell or a population of genetic engineered human cells having at least one genetic modification.

In one embodiment, any modified synthetic nucleic acid or RNP described herein is used in the ex vivo targeted genome editing of the ELANE gene in a progenitor cell purpose.

In one embodiment, any modified synthetic nucleic acid or RNP described herein is used in an ex vivo method of producing a progenitor cell or a population of progenitor cells wherein the cells, or the differentiated progeny therefrom, have decreased ELANE mRNA or protein expression. One skilled in the art can determine if ELANE mRNA or protein expression using standard techniques, e.g., via PCR-based assays or western-blotting to measure the mRNA or protein level, respectively.

In one embodiment, any modified synthetic nucleic acid or RNP described herein is used an ex vivo method of producing an isolated genetic engineered human cell or a population of progenitor cells having at least one genetic modification. One skilled in the art can determine if a genetic modification is present in the genome, e.g., by sequencing the cell or population thereof and comparing the sequence to a reference sequence. As used herein, “reference” sequence refers to the sequence of an otherwise identical cell that is not contacted by a composition described herein.

In one embodiment, the isolated progenitor cell is an isolated human cell. In one embodiment, the isolated human cell is a hematopoietic progenitor cell or a hematopoietic stem cell. In another embodiment, the isolated human cell is an embryonic stem cell, a somatic stem cell, a progenitor cell, or a bone marrow cell. In one embodiment, the hematopoietic progenitor is a cell of the erythroid lineage.

In one embodiment, the isolated progenitor cell or isolated cell is an induced pluripotent stem cell.

In one embodiment, the progenitor cell or human cell acquires at least one genetic modification. Exemplary modifications include a deletion, insertion or substitution of the genetic sequence of the cell, as compared to a wild-type sequence. One skilled in the art can determine if a genetic modification is present in the genome, e.g., by sequencing the cell or population thereof and comparing the sequence to a reference sequence. As used herein, “reference” sequence refers to the sequence of an otherwise identical cell that is not contacted by a composition described herein.

Another aspect provided herein is a method of treating a disease associated with abnormal ELANE gene expression, the method comprising, administering to a subject in need thereof a gene editing agent that targets the ELANE gene, wherein the edited ELANE gene comprises at least one mutation selected from the group consisting of a loss-of-function mutation, a through stop-gain mutation, a frameshift mutation, a premature termination codon, and a splicing mutation that encodes a premature termination codon. One skilled in the art can assess abnormal ELANE gene expression can be determined using standard techniques, e.g., via genome sequencing, or using a functional assay, for example, assessing NE expression. One skilled in the art can determine if a mutation has occurred, e.g., comparing the sequence of the edited ELANE to the wild-type, unedited sequence (e.g., not contacted by a gene editing agent described herein).

In one embodiment, the disease is selected from the group consisting of chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis, chronic kidney disease, cyclic neutropenia, and severe congential neutropenia.

In one embodiment, the gene editing agent is selected from the group consisting of clustered regularly interspaced short palindromic repeats (CRISPR), Transcription activator-like effector nucleases (TALEN), Meganuclease, Zinc finger nucleases, Homologous recombination, and deaminase fusion proteins. As used herein, “gene editing agent” refers to any agent designed to target the NDA sequence of a gene, e.g., ELANE, and induce at least one mutation, such as a base pair deletion, insertation, or substitution.

In one embodiment, the gene editing agent is any modified synthetic nucleic acid, composition, or RNP complex described herein.

In one embodiment, the at least one mutation results in induction of nonsense-mediated decay, translational repression, or knockdown of transcript. One skilled in the art can determine is decay, repression or knockdown has occurred using known techniques, e.g., by assessing ELANE gene and/or protein levels using PCR-based assays or westernblotting, respectively.

In one embodiment, the abnormal ELANE gene expression results in abnormal neutrophil elastase protein. As used herein, “abnormal” refers to modulated function or levels. For example, abnormal neutrophil elastase protein can refer to neutrophil elastase protein that no longer functions in a normal manner, such as wold-type neutrophil elastase protein. Alternatively, abnormal neutrophil elastase protein can refer to increased or descreased levels of neutrophil elastase protein in the cell, as compared to wild-type levels of neutrophil elastase protein.

### Compositions

One aspect provides a composition comprising any of the modified synthetic nucleic acid molecule described herein. In one embodiment, the composition further comprises a DNA-targeting endonuclease Cas protein. In one embodiment, the Cas protein is Cas9.

Another aspect provides a composition comprising any of the RNP complexes described herein

In one embodiment, the compositions described herein further comprise a pharmaceutically acceptable carrier. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically, such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

In various embodiment, the composition is a pharmaceutical composition.

In one embodiment, any composition described herein is used in the ex vivo targeted genome editing of the ELANE gene in a progenitor cell purpose.

In one embodiment, any composition described herein is used in an ex vivo method of producing a progenitor cell or a population of progenitor cells wherein the cells, or the differentiated progeny therefrom, have decreased ELANE mRNA or protein expression. One skilled in the art can determine if ELANE mRNA or protein expression using standard techniques, e.g., via PCR-based assays or western-blotting to measure the mRNA or protein level, respectively.

In one embodiment, any composition described herein is used an ex vivo method of producing an isolated genetic engineered human cell or a population of progenitor cells having at least one genetic modification. One skilled in the art can determine if a genetic modification is present in the genome, e.g., by sequencing the cell or population thereof and comparing the sequence to a reference sequence. As used herein, “reference” sequence refers to the sequence of an otherwise identical cell that is not contacted by a composition described herein.

In one embodiment, the isolated progenitor cell or isolated cell is a hematopoietic progenitor cell or a hematopoietic stem cell. In one embodiment, the hematopoietic progenitor is a cell of the erythroid lineage.

In one embodiment, the isolated progenitor cell or isolated cell is an induced pluripotent stem cell.

In one embodiment, the progenitor cell or human cell acquires at least one genetic modification. Exemplary modifications include a deletion, insertion or substitution of the genetic sequence of the cell, as compared to the wild-type sequence. One skilled in the art can determine if a genetic modification is present in the genome, e.g., by sequencing the cell or population thereof and comparing the sequence to a reference sequence. As used herein, “reference” sequence refers to the sequence of an otherwise identical cell that is not contacted by a composition described herein.

### Electroporation

In various embodiments, any modified synthetic nucleic acid, RNP complex, or composition thereof described herein is placed in a cell, e.g., for its cellular expression, via electroporation. In a preferred embodiment, the RNP complex is electroporated in a solution having glycerol, e.g., 2-4% glycerol.

It was found herein that electroporation of cells, as described herein, in a solution comprising glycerol increases the cell viability following electroporation, as compared to electroporation in a solution that does not comprise glycerol. Accordingly, in one embodiment, the step of electroporation is performed in a solution comprising glycerol. In one embodiment, the solution (e.g., a suitable buffer used for electroporation) comprises at least 1% glycerol. In one embodiment, the solution comprises 2-4% glycerol. In one embodiment, the solution comprising less than 1%, at least 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more glycerol. In one embodiment, the amount of glycerol in the solution ranges from 2-4%. In one embodiment, the amount of glycerol in the solution ranges from 1-2%, 1-3%, 1-4%, 1-5%, 1-6%, 1-7%, 1-8%, 1-9%, 1-10%, 1-20%, 1-30%, 5-10%, 5-15%, 5-20%, 5-25%, 5-30%, 10-15%, 10-20%, 10-25%, 10-30%, 15-20%, 15-25%, 15-30%, 20-25%, 20-30%, 25-30%, 2-5%, 2-6%, 2-7%, 2-8%, 2-9%, 2-10%, 3-4%, 3-5%, 3-7%, 3-8%, 3-9%, 3-10%, 4-5%, 4-6%. 4-7%. 4-8%. 4-9%, 4-10%, 5-6%, 5-7%, 5-8%, 5-9%, 6-7%, 6-8%, 6-9%, 6-10%, -8%, 7-9%, 8-9%, 8-10%, or 9-10%. Glycerol can be purified glycerol or unpurified glycerol. Glycerol can be naturally occurring or synthesized. Glycerol can be derived from various processes known in the art, e.g., from plant or animal sources in which it occurs as triglerides, or propylene. In one embodiment, the solution comprises a glycerol derivative. Glycerol derivatives are further described in, e.g., U.S. Patent Application US2008/029360, which is incorporated herein by reference in its entirety.

In various embodiment, expression of any modified synthetic nucleic acid or RNP complex, or composition thereof described herein results in the inhibition of expression and/or activity (e.g., translation of a polypeptide encoded by the ELANE gene) of the ELANE gene. In one embodiment, expression and/or activity (e.g., translation of a polypeptide) of the ELANE gene in the cell is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell, e.g., a cell that is not treated in any method or reagent disclosed herein. In one embodiment, the level of the polypeptide encoded by of the ELANE gene, i.e., neutrophil elastase, in the cell is at least 5% lower is at least 10% lower, at least 20% lower, at least 30% lower, at least 40% lower, at least 50% lower, at least 60% lower, at least 70% lower, at least 80% lower, at least 90% lower, at least 1-fold lower, at least 2-fold lower, at least 5-fold lower, at least 10 fold lower, at least 100 fold lower, at least 1000-fold lower, or more compared to a control cell that is not treated in any method disclosed herein. One skilled in the art can determine if the level of the ELANE gene, or the polypeptide encoded therefrom, is reduced in a cell expressing any of the reagents described herein using standard techniques, e.g., via PCR-based assays or western-blotting to measure the mRNA or protein level, respectively. Measuring the level of polypeptide encoded by the ELANE gene can determine the level of activity of the ELANE gene.

In one embodiment, the cells obtained after electroporation of the modified synthetic nucleic acid, RNP complex, or composition thereof described herein can be cryopreserved till they are needed for administration into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells or iPSCs are autologous to the mammal, meaning the cells are derived from the same mammal. In another of the embodiment of the described method, the hematopoietic progenitor or stem cells or iPSCs are non-autologous to the mammal, meaning the cells are not derived from the same mammal, but another mammal of the same species. For example, the mammal is a human.

### Methods for Producing a Progenitor Cell

One aspect provided herein is a method for producing a progenitor cell or a population of progenitor cells having decreased ELANE mRNA or protein expression comprising contacted an isolated progenitor cell with an effective amount of any modified synthetic nucleic acid, RNP complex, or any composition thereof, whereby the contacted cells or the differentiated progeny cells therefrom have decreased ELANE mRNA or protein expression.

In one embodiment, the contacted cell having decreased ELANE mRNA or protein expression comprises at least one genetic mutation, e.g., a substitution, deletion, or addition, as compared to the wild-type sequence.

In one embodiment, the isolated progenitor cell is a hematopoietic progenitor cell or a hematopoietic stem cell. In one embodiment, the hematopoietic progenitor is a cell of the erythroid lineage. Methods of isolating hematopoietic progenitor cell are well known in the art, e.g., by flow cytometric purification of CD34+ or CD133+ cells, microbeads conjugated with antibodies against CD34 or CD133, markers of hematopoietic progenitor cell. Commercial kits are also available, e.g., MACS® Technology CD34 MicroBead Kit, human, and CD34 MultiSort Kit, human, and STEMCELL™ Technology EasySep™ Mouse Hematopoietic Progenitor Cell Enrichment Kit.

In another embodiment, the hematopoietic stem cells, hematopoietic progenitor cells, embryonic stem cells, somatic stem cells, or progenitor cells are collected from peripheral blood, cord blood, chorionic villi, amniotic fluid, placental blood, or bone marrow.

In one embodiment, isolated progenitor cell is an iPSCs described herein.

In one embodiment, the contacted cell or its progeny is administered to the mammal.

In further embodiment of any one treatment method described, the method comprises chemotherapy and/or radiation therapy to remove or reduced the endogenous hematopoietic progenitor or stem cells in the mammal.

In one embodiment, the contacted cells having at least one genetic modification can be cryopreserved and stored until the cells are needed for administration into a mammal.

In one embodiment, the contacted cells having at least one genetic modification can be cultured ex vivo to expand or increase the number of cells prior to storage, e.g., by cryopreservation, or prior to use, e.g., transplanted into a recipient mammal, e.g., a patient.

In one embodiment of this aspect and all other aspects described herein, the contacted population of hematopoietic progenitor or stem cells having decreased ELANE expression is cryopreserved and stored or reintroduced into the mammal. In another embodiment, the cryopreserved population of hematopoietic progenitor or stem cells having decreased ELANE expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs described herein.

In another embodiment, the cryopreserved population of contacted cell, e.g., hematopoietic progenitor or stem cells having decreased ELANE expression is thawed and then reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs derived from the mammal. In any embodiment of the method, the method further comprises selecting a mammal in need of decreased ELANE expression; a subject having or suspected of having SCN.

In further embodiment of this method, the population of hematopoietic progenitor or stem cells with genetic modification or targeted gene editing in the genomic DNA and having reduced ELANE expression is cryopreserved and stored or reintroduced into the mammal. In further embodiment of this method, the method comprises chemotherapy and/or radiation therapy to remove or reduced the endogenous hematopoietic progenitor or stem cells in the mammal. In any of the embodiment of the described method, the hematopoietic progenitor or stem cells can be substituted with an iPSCs derived from the mammal. In any embodiment of the method, the method further comprises selecting a mammal in need of decreased ELANE expression, e.g., a subject having or suspected of having SCN. In any of the embodiment of the described method, the hematopoietic progenitor or stem cells can be substituted with an iPSCs described herein. In any of the embodiment of the described method, the hematopoietic progenitor or stem cells or iPSCs are analogous to the mammal, meaning the cells are derived from the same mammal. In another of the embodiment of the described method, the hematopoietic progenitor or stem cells or iPSCs are non-analogous to the mammal, meaning the cells are not derived from the same mammal, but another mammal of the same species. For example, the mammal is a human.

One aspect herein is a population of genetically edited human cells having at least one genetic modification resulting in decreased ELANE mRNA or protein expression. In one embodiment, the genetically edited human cells are isolated.

One aspect herein is a population of genetically edited progenitor cells having at least one genetic modification resulting in decreased ELANE mRNA or protein expression.

Various aspects herein provide a composition comprising genetically edited human cells having at least one genetic modification resulting in decreased ELANE mRNA or protein expression, or genetically edited progenitor cells having at least one genetic modification resulting in decreased ELANE mRNA or protein expression.

In one embodiment of use of the composition described herein, the cells of any compositions described are autologous, to the mammal who is the recipient of the cells in a transplantation procedure, i.e., the cells of the composition are derived or harvested from the mammal prior to any described modification.

In one embodiment of use of the composition described herein, the cells of any compositions described are non-autologous to the mammal who is the recipient of the cells in a transplantation procedure, i.e., the cells of the composition are not derived or harvested from the mammal prior to any described modification.

In one embodiment, a dose of genetically modified cells is delivered to a subject intravenously. In one embodiment, genetically modified hematopoietic cells are intravenously administered to a subject.

In one embodiment, patients receive a dose of genetically modified cells, e.g., hematopoietic stem cells, of about 1×10⁵ cells/kg, about 5×10⁵ cells/kg, about 1×10⁶ cells/kg, about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 5×10⁷ cells/kg, about 1×10⁸ cells/kg, or more in one single intravenous dose. In certain embodiments, patients receive a dose of genetically modified cells, e.g., hematopoietic stem cells described herein or genetic engineered cells described herein or progeny thereof, of at least 1×10⁵ cells/kg, at least 5×10⁵ cells/kg, at least 1×10⁶ cells/kg, at least 2×10⁶ cells/kg, at least 3×10⁶ cells/kg, at least 4×10⁶ cells/kg, at least 5×10⁶ cells/kg, at least 6×10⁶ cells/kg, at least 7×10⁶ cells/kg, at least 8×10⁶ cells/kg, at least 9×10⁶ cells/kg, at least 1×10⁷ cells/kg, at least 5×10⁷ cells/kg, at least 1×10⁸ cells/kg, or more in one single intravenous dose.

In one embodiment, patients receive a dose of genetically modified cells, e.g., hematopoietic stem cells, of about 1×10⁵ cells/kg to about 1×10⁸ cells/kg, about 1×10⁶ cells/kg to about 1×10⁸ cells/kg,×10⁶ cells/kg to about 8×10⁶ cells/kg, about 2×10⁶ cells/kg to about 5×10⁶ cells/kg, about 3×10⁶ cells/kg to about 5×10⁶ cells/kg, about 3×10⁶ cells/kg to about 4×10⁸ cells/kg, or any intervening dose of cells/kg.

In various embodiments, the methods described here provide more robust and safe gene therapy than existing methods and comprise administering a population or dose of cells comprising about 5% transduced/ genetically modified cells, about 10% transduced/genetically modified cells, about 15% transduced/genetically modified cells, about 20% transduce/genetically modified d cells, about 25% transduced/genetically modified cells, about 30% transduced/genetically modified cells, about 35% transduced/genetically modified cells, about 40% transduced/genetically modified cells, about 45% transduced/genetically modified cells, or about 50% transduce/genetically modified cells, to a subject.

In one embodiment, the invention provides genetically modified cells, such as a stem cell, e.g., hematopoietic stem cell, with the potential to expand or increase a population of erythroid cells. Hematopoietic stem cells are the origin of erythroid cells and thus, are preferred.

In one embodiment, the hematopoietic stem cell or hematopoietic progenitor cell being contacted is of the erythroid lineage.

In one embodiment, the contacted hematopoietic stem cells described herein or genetic engineered cells described herein or the progeny cells thereof are treated ex vivo with prostaglandin E2 and/or antioxidant N-acetyl-L-cysteine (NAC) to promote subsequent engraftment in a recipient subject.

Engraftment analysis was performed 4, 8 and 12 weeks post transplantation in peripheral blood and bone marrow. For example, harvest a sample of blood from these locations and determine the ELANE expression by any method known in the art.

In one embodiment, the contact cell is a human cell. In one embodiment, the contacted cell is an embryonic stem cell, a somatic stem cell, a progenitor cell, a bone marrow cell, a hematopoietic stem cell, or a hematopoietic progenitor cell.

In one embodiment, the method comprises, prior to contacting, obtaining a sample or a population of embryonic stem cells, somatic stem cells, progenitor cells, bone marrow cells, hematopoietic stem cells, or hematopoietic progenitor cells from the subject.

In one embodiment, the cells that is contacted with a nucleic acid molecule describe herein, or a composition describe herein comprising a nucleic acid molecule.

In one embodiment, the embryonic stem cells, somatic stem cells, progenitor cells, bone marrow cells, hematopoietic stem cells, hematopoietic progenitor cells are isolated from the host subject, transfected, cultured (optional), and transplanted back into the same host, i. e. an autologous cell transplant. In another embodiment, the embryonic stem cells, somatic stem cells, progenitor cells, bone marrow cells, hematopoietic stem cells, or hematopoietic progenitor cells are isolated from a donor who is an HLA-type match with a host (recipient) who is diagnosed with or at risk of developing a SCN. Donor-recipient antigen type-matching is well known in the art. The HLA-types include HLA-A, HLA-B, HLA-C, and HLA-D. These represent the minimum number of cell surface antigen matching required for transplantation. That is the transfected cells are transplanted into a different host, i.e., allogeneic to the recipient host subject. The donor's or subject's embryonic stem cells, somatic stem cells, progenitor cells, bone marrow cells, hematopoietic stem cells, or hematopoietic progenitor cells can be contacted (electroporated) with a nucleic acid molecule described herein, the contacted cells are culture expanded, and then transplanted into the host subject. In one embodiment, the transplanted cells engraft in the host subject. The transfected cells can also be cryopreserved after transfected and stored, or cryopreserved after cell expansion and stored.

In one aspect of any method, the embryonic stem cell, somatic stem cell, progenitor cell, bone marrow cell, hematopoietic stem cell, or hematopoietic progenitor cell is autologous or allogeneic to the subject.

### Hematopoietic Progenitor Cells

In one embodiment, the hematopoietic progenitor cell is contacted ex vivo or in vitro. In a specific embodiment, the cell being contacted is a cell of the erythroid lineage. In one embodiment, the cell composition comprises cells having decreased ELANE expression.

“Hematopoietic progenitor cell” as the term is used herein, refers to cells of a stem cell lineage that give rise to all the blood cell types including the myeloid (monocytes and macrophages, neutrophils, basophils, eosinophils, erythrocytes, megakaryocytes/platelets, dendritic cells), and the lymphoid lineages (T-cells, B-cells, NK-cells). A “cell of the erythroid lineage” indicates that the cell being contacted is a cell that undergoes erythropoiesis such that upon final differentiation it forms an erythrocyte or red blood cell (RBC). Such cells belong to one of three lineages, erythroid, lymphoid, and myeloid, originating from bone marrow hematopoietic progenitor cells. Upon exposure to specific growth factors and other components of the hematopoietic microenvironment, hematopoietic progenitor cells can mature through a series of intermediate differentiation cellular types, all intermediates of the erythroid lineage, into RBCs. Thus, cells of the “erythroid lineage”, as the term is used herein, comprise hematopoietic progenitor cells, rubriblasts, prorubricytes, erythroblasts, metarubricytes, reticulocytes, and erythrocytes.

In some embodiment, the hematopoietic progenitor cell has at least one of the cell surface marker characteristic of hematopoietic progenitor cells: CD34+, CD59+, Thy1/CD90+, CD381o/-, and C-kit/CD117+. Preferably, the hematopoietic progenitor cells have several of these markers.

In some embodiments, the hematopoietic progenitor cells of the erythroid lineage have the cell surface marker characteristic of the erythroid lineage: CD71 and Ter119.

Stem cells, such as hematopoietic progenitor cells, are capable of proliferation and giving rise to more progenitor cells having the ability to generate a large number of mother cells that can in turn give rise to differentiated or differentiable daughter cells. The daughter cells themselves can be induced to proliferate and produce progeny that subsequently differentiate into one or more mature cell types, while also retaining one or more cells with parental developmental potential. The term “stem cell” refers then, to a cell with the capacity or potential, under particular circumstances, to differentiate to a more specialized or differentiated phenotype, and which retains the capacity, under certain circumstances, to proliferate without substantially differentiating. In one embodiment, the term progenitor or stem cell refers to a generalized mother cell whose descendants (progeny) specialize, often in different directions, by differentiation, e.g., by acquiring completely individual characters, as occurs in progressive diversification of embryonic cells and tissues. Cellular differentiation is a complex process typically occurring through many cell divisions. A differentiated cell may derive from a multipotent cell which itself is derived from a multipotent cell, and so on. While each of these multipotent cells may be considered stem cells, the range of cell types each can give rise to may vary considerably. Some differentiated cells also have the capacity to give rise to cells of greater developmental potential. Such capacity may be natural or may be induced artificially upon treatment with various factors. In many biological instances, stem cells are also “multipotent” because they can produce progeny of more than one distinct cell type, but this is not required for “stem-ness.” Self-renewal is the other classical part of the stem cell definition, and it is essential as used in this document. In theory, self-renewal can occur by either of two major mechanisms. Stem cells may divide asymmetrically, with one daughter retaining the stem state and the other daughter expressing some distinct other specific function and phenotype. Alternatively, some of the stem cells in a population can divide symmetrically into two stems, thus maintaining some stem cells in the population as a whole, while other cells in the population give rise to differentiated progeny only. Generally, “progenitor cells” have a cellular phenotype that is more primitive (i.e., is at an earlier step along a developmental pathway or progression than is a fully differentiated cell). Often, progenitor cells also have significant or very high proliferative potential. Progenitor cells can give rise to multiple distinct differentiated cell types or to a single differentiated cell type, depending on the developmental pathway and on the environment in which the cells develop and differentiate.

In the context of cell ontogeny, the adjective “differentiated”, or “differentiating” is a relative term. A “differentiated cell” is a cell that has progressed further down the developmental pathway than the cell it is being compared with. Thus, stem cells can differentiate to lineage-restricted precursor cells (such as a hematopoietic progenitor cell), which in turn can differentiate into other types of precursor cells further down the pathway (such as an erythrocyte precursor), and then to an end-stage differentiated cell, such as an erythrocyte, which plays a characteristic role in a certain tissue type, and may or may not retain the capacity to proliferate further.

### Induced Pluripotent Stem Cells

In some embodiments, the genetic engineered human cells described herein are derived from isolated pluripotent stem cells. An advantage of using iPSCs is that the cells can be derived from the same subject to which the progenitor cells are to be administered. That is, a somatic cell can be obtained from a subject, reprogrammed to an induced pluripotent stem cell, and then re-differentiated into a hematopoietic progenitor cell to be administered to the subject (e.g., autologous cells). Since the progenitors are essentially derived from an autologous source, the risk of engraftment rejection or allergic responses is reduced compared to the use of cells from another subject or group of subjects. In some embodiments, the hematopoietic progenitors are derived from non-autologous sources. In addition, the use of iPSCs negates the need for cells obtained from an embryonic source. Thus, in one embodiment, the stem cells used in the disclosed methods are not embryonic stem cells.

Although differentiation is generally irreversible under physiological contexts, several methods have been recently developed to reprogram somatic cells to induced pluripotent stem cells. Exemplary methods are known to those of skill in the art and are described briefly herein below.

As used herein, the term “reprogramming” refers to a process that alters or reverses the differentiation state of a differentiated cell (e.g., a somatic cell). Stated another way, reprogramming refers to a process of driving the differentiation of a cell backwards to a more undifferentiated or more primitive type of cell. It should be noted that placing many primary cells in culture can lead to some loss of fully differentiated characteristics. Thus, simply culturing such cells included in the term differentiated cells does not render these cells non-differentiated cells (e.g., undifferentiated cells) or pluripotent cells. The transition of a differentiated cell to pluripotency requires a reprogramming stimulus beyond the stimuli that lead to partial loss of differentiated character in culture. Reprogrammed cells also have the characteristic of the capacity of extended passaging without loss of growth potential, relative to primary cell parents, which generally have capacity for only a limited number of divisions in culture.

The cell to be reprogrammed can be either partially or terminally differentiated prior to reprogramming. In some embodiments, reprogramming encompasses complete reversion of the differentiation state of a differentiated cell (e.g., a somatic cell) to a pluripotent state or a multipotent state. In some embodiments, reprogramming encompasses complete or partial reversion of the differentiation state of a differentiated cell (e.g., a somatic cell) to an undifferentiated cell (e.g., an embryonic-like cell). Reprogramming can result in expression of particular genes by the cells, the expression of which further contributes to reprogramming. In certain embodiments described herein, reprogramming of a differentiated cell (e.g., a somatic cell) causes the differentiated cell to assume an undifferentiated state (e.g., is an undifferentiated cell). The resulting cells are referred to as “reprogrammed cells,” or “induced pluripotent stem cells (iPSCs or iPS cells).”

Reprogramming can involve alteration, e.g., reversal, of at least some of the heritable patterns of nucleic acid modification (e.g., methylation), chromatin condensation, epigenetic changes, genomic imprinting, etc., that occur during cellular differentiation. Reprogramming is distinct from simply maintaining the existing undifferentiated state of a cell that is already pluripotent or maintaining the existing less than fully differentiated state of a cell that is already a multipotent cell (e.g., a hematopoietic stem cell). Reprogramming is also distinct from promoting the self-renewal or proliferation of cells that are already pluripotent or multipotent, although the compositions and methods described herein can also be of use for such purposes, in some embodiments.

The specific approach or method used to generate pluripotent stem cells from somatic cells (broadly referred to as “reprogramming”) is not critical to the claimed invention. Thus, any method that re-programs a somatic cell to the pluripotent phenotype would be appropriate for use in the methods described herein.

Reprogramming methodologies for generating pluripotent cells using defined combinations of transcription factors have been described induced pluripotent stem cells. Yamanaka and Takahashi converted mouse somatic cells to ES cell-like cells with expanded developmental potential by the direct transduction of Oct4, Sox2, Klf4, and c-Myc (Takahashi and Yamanaka, 2006). iPSCs resemble ES cells as they restore the pluripotency-associated transcriptional circuitry and much of the epigenetic landscape. In addition, mouse iPSCs satisfy all the standard assays for pluripotency: specifically, in vitro differentiation into cell types of the three germ layers, teratoma formation, contribution to chimeras, germline transmission (Maherali and Hochedlinger, 2008), and tetraploid complementation (Woltjen et al., 2009).

Subsequent studies have shown that human iPS cells can be obtained using similar transduction methods (Lowry et al., 2008; Park et al., 2008; Takahashi et al., 2007; Yu et al., 2007b), and the transcription factor trio, OCT4, SOX2, and NANOG, has been established as the core set of transcription factors that govern pluripotency (Jaenisch and Young, 2008). The production of iPS cells can be achieved by the introduction of nucleic acid sequences encoding stem cell-associated genes into an adult, somatic cell, historically using viral vectors.

iPS cells can be generated or derived from terminally differentiated somatic cells, as well as from adult stem cells, or somatic stem cells. That is, a non-pluripotent progenitor cell can be rendered pluripotent or multipotent by reprogramming. In such instances, it may not be necessary to include as many reprogramming factors as required to reprogram a terminally differentiated cell. Further, reprogramming can be induced by the non-viral introduction of reprogramming factors, e.g., by introducing the proteins themselves, or by introducing nucleic acids that encode the reprogramming factors, or by introducing messenger RNAs that upon translation produce the reprogramming factors (see e.g., Warren et al., Cell Stem Cell, 2010 Nov. 5;7(5):618-30). Reprogramming can be achieved by introducing a combination of nucleic acids encoding stem cell-associated genes including, for example Oct-4 (also known as Oct-3/4 or Pouf51), Sox1, Sox2, Sox3, Sox 15, Sox 18, NANOG, Klf1, Klf2, Klf4, Klf5, NR5A2, c-Myc, 1-Myc, n-Myc, Rem2, Tert, and LIN28. In one embodiment, reprogramming using the methods and compositions described herein can further comprise introducing one or more of Oct-3/4, a member of the Sox family, a member of the Klf family, and a member of the Myc family to a somatic cell. In one embodiment, the methods and compositions described herein further comprise introducing one or more of each of Oct 4, Sox2, Nanog, c-MYC and Klf4 for reprogramming. As noted above, the exact method used for reprogramming is not necessarily critical to the methods and compositions described herein. However, where cells differentiated from the reprogrammed cells are to be used in, e.g., human therapy, in one embodiment the reprogramming is not effected by a method that alters the genome. Thus, in such embodiments, reprogramming is achieved, e.g., without the use of viral or plasmid vectors.

The efficiency of reprogramming (i.e., the number of reprogrammed cells) derived from a population of starting cells can be enhanced by the addition of various small molecules as shown by Shi, Y., et al (2008) Cell-Stem Cell 2:525-528, Huangfu, D., et al (2008) Nature Biotechnology 26(7):795-797, and Marson, A., et al (2008) Cell-Stem Cell 3:132-135. Thus, an agent or combination of agents that enhance the efficiency or rate of induced pluripotent stem cell production can be used in the production of patient-specific or disease-specific iPSCs. Some non-limiting examples of agents that enhance reprogramming efficiency include soluble Wnt, Wnt conditioned media, BIX-01294 (a G9a histone methyltransferase), PD0325901 (a MEK inhibitor), DNA methyltransferase inhibitors, histone deacetylase (HDAC) inhibitors, valproic acid, 5′-azacytidine, dexamethasone, suberoylanilide, hydroxamic acid (SAHA), vitamin C, and trichostatin (TSA), among others.

Other non-limiting examples of reprogramming enhancing agents include: Suberoylanilide Hydroxamic Acid (SAHA (e.g., MK0683, vorinostat) and other hydroxamic acids), BML-210, Depudecin (e.g., (−)-Depudecin), HC Toxin, Nullscript (4-(1,3-Dioxo-1H,3H-benzo[de]isoquinolin-2-yl)-N-hydroxybutanamide), Phenylbutyrate (e.g., sodium phenylbutyrate) and Valproic Acid ((VPA) and other short chain fatty acids), Scriptaid, Suramin Sodium, Trichostatin A (TSA), APHA Compound 8, Apicidin, Sodium Butyrate, pivaloyloxymethyl butyrate (Pivanex, AN-9), Trapoxin B, Chlamydocin, Depsipeptide (also known as FR901228 or FK228), benzamides (e.g., CI-994 (e.g., N-acetyl dinaline) and MS-27-275), MGCD0103, NVP-LAQ-824, CBHA (m-carboxycinnaminic acid bishydroxamic acid), JNJ16241199, Tubacin, A-161906, proxamide, oxamflatin, 3-Cl-UCHA (e.g., 6-(3-chlorophenylureido)caproic hydroxamic acid), AOE (2-amino-8-oxo-9,10-epoxydecanoic acid), CHAP31 and CHAP 50. Other reprogramming enhancing agents include, for example, dominant negative forms of the HDACs (e.g., catalytically inactive forms), siRNA inhibitors of the HDACs, and antibodies that specifically bind to the HDACs. Such inhibitors are available, e.g., from BIOMOL International, Fukasawa, Merck Biosciences, Novartis, Gloucester Pharmaceuticals, Aton Pharma, Titan Pharmaceuticals, Schering AG, Pharmion, MethylGene, and Sigma Aldrich.

To confirm the induction of pluripotent stem cells for use with the methods described herein, isolated clones can be tested for the expression of a stem cell marker. Such expression in a cell derived from a somatic cell identifies the cells as induced pluripotent stem cells. Stem cell markers can be selected from the non-limiting group including SSEA3, SSEA4, CD9, Nanog, Fbx15, Ecat1, Esg1, Eras, Gdf3, Fgf4, Cripto, Dax1, Zpf296, Slc2a3, Rex1, Utf1, and Nat1. In one embodiment, a cell that expresses Oct4 or Nanog is identified as pluripotent. Methods for detecting the expression of such markers can include, for example, RT-PCR and immunological methods that detect the presence of the encoded polypeptides, such as Western blots or flow cytometric analyses. In some embodiments, detection does not involve only RT-PCR, but also includes detection of protein markers. Intracellular markers may be best identified via RT-PCR, while cell surface markers are readily identified, e.g., by immunocytochemistry.

The pluripotent stem cell character of isolated cells can be confirmed by tests evaluating the ability of the iPSCs to differentiate to cells of each of the three germ layers. As one example, teratoma formation in nude mice can be used to evaluate the pluripotent character of the isolated clones. The cells are introduced to nude mice and histology and/or immunohistochemistry is performed on a tumor arising from the cells. The growth of a tumor comprising cells from all three germ layers, for example, further indicates that the cells are pluripotent stem cells.

### Somatic Cells for Reprogramming

Somatic cells, as that term is used herein, refer to any cells forming the body of an organism, excluding germline cells. Every cell type in the mammalian body—apart from the sperm and ova, the cells from which they are made (gametocytes) and undifferentiated stem cells—is a differentiated somatic cell. For example, internal organs, skin, bones, blood, and connective tissue are all made up of differentiated somatic cells.

Additional somatic cell types for use with the compositions and methods described herein include: a fibroblast (e.g., a primary fibroblast), a muscle cell (e.g., a myocyte), a cumulus cell, a neural cell, a mammary cell, a hepatocyte and a pancreatic islet cell. In some embodiments, the somatic cell is a primary cell line or is the progeny of a primary or secondary cell line. In some embodiments, the somatic cell is obtained from a human sample, e.g., a hair follicle, a blood sample, a biopsy (e.g., a skin biopsy or an adipose biopsy), a swab sample (e.g., an oral swab sample), and is thus a human somatic cell.

Some non-limiting examples of differentiated somatic cells include, but are not limited to, epithelial, endothelial, neuronal, adipose, cardiac, skeletal muscle, immune cells, hepatic, splenic, lung, circulating blood cells, gastrointestinal, renal, bone marrow, and pancreatic cells. In some embodiments, a somatic cell can be a primary cell isolated from any somatic tissue including, but not limited to brain, liver, gut, stomach, intestine, fat, muscle, uterus, skin, spleen, endocrine organ, bone, etc. Further, the somatic cell can be from any mammalian species, with non-limiting examples including a murine, bovine, simian, porcine, equine, ovine, or human cell. In some embodiments, the somatic cell is a human somatic cell.

When reprogrammed cells are used for generation of hematopoietic progenitor cells to be used in the therapeutic treatment of disease, it is desirable, but not required, to use somatic cells isolated from the patient being treated. For example, somatic cells involved in diseases, and somatic cells participating in therapeutic treatment of diseases and the like can be used. In some embodiments, a method for selecting the reprogrammed cells from a heterogeneous population comprising reprogrammed cells and somatic cells they were derived or generated from can be performed by any known means. For example, a drug resistance gene or the like, such as a selectable marker gene can be used to isolate the reprogrammed cells using the selectable marker as an index.

Reprogrammed somatic cells as disclosed herein can express any number of pluripotent cell markers, including: alkaline phosphatase (AP); ABCG2; stage specific embryonic antigen-1 (SSEA-1); SSEA-3; SSEA-4; TRA-1-60; TRA-1-81; Tra-2-49/6E; ERas/ECAT5, E-cadherin; β-III-tubulin; α-smooth muscle actin (α-SMA); fibroblast growth factor 4 (Fgf4), Cripto, Daxl; zinc finger protein 296 (Zfp296); N-acetyltransferase-1 (Nati); (ES cell associated transcript 1 (ECAT1); ESG1/DPPA5/ECAT2; ECAT3; ECAT6; ECAT7; ECAT8; ECAT9; ECAT10; ECAT15-1; ECAT15-2; Fth117; Sal14; undifferentiated embryonic cell transcription factor (Utf1); Rex1; p53; G3PDH; telomerase, including TERT; silent X chromosome genes; Dnmt3a; Dnmt3b; TRIM28; F-box containing protein 15 (Fbx15); Nanog/ECAT4; Oct3/4; Sox2; Klf4; c-Myc; Esrrb; TDGF1; GABRB3; Zfp42, FoxD3; GDF3; CYP25A1; developmental pluripotency-associated 2 (DPPA2); T-cell lymphoma breakpoint 1 (Tcl1); DPPA3/Stella; DPPA4; other general markers for pluripotency, etc. Other markers can include Dnmt3L; Sox15;Stat3; Grb2; β-catenin, and Bmi1. Such cells can also be characterized by the down-regulation of markers characteristic of the somatic cell from which the induced pluripotent stem cell is derived.

### Cell Compositions

The methods of administering human hematopoietic progenitor cells or genetic engineered cells described herein or their progeny to a subject as described herein involve the use of therapeutic compositions comprising hematopoietic progenitor cells. Therapeutic compositions contain a physiologically tolerable carrier together with the cell composition and optionally at least one additional bioactive agent as described herein, dissolved or dispersed therein as an active ingredient. In a preferred embodiment, the therapeutic composition is not substantially immunogenic when administered to a mammal or human patient for therapeutic purposes, unless so desired.

In general, the hematopoietic progenitor cells described herein or genetic engineered cells described herein or their progeny are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the hematopoietic progenitor cells as described herein using routine experimentation.

A cell composition can also be emulsified or presented as a liposome composition, provided that the emulsification procedure does not adversely affect cell viability. The cells and any other active ingredient can be mixed with excipients which are pharmaceutically acceptable and compatible with the active ingredient and in amounts suitable for use in the therapeutic methods described herein.

Additional agents included in a cell composition as described herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions as described herein that is effective in the treatment of a particular disorder or condition will depend on the nature of the disorder or condition, and can be determined by standard clinical techniques.

In some embodiments, the compositions of isolated genetic engineered cells described further comprises a pharmaceutically acceptable carrier. In one embodiment, the pharmaceutically acceptable carrier does not include tissue or cell culture media.

In some embodiments, the compositions of modified synthetic nucleic acid molecules described further comprises a pharmaceutically acceptable carrier. In one embodiment, the pharmaceutically acceptable carrier does not include tissue or cell culture media.

In some embodiments, the compositions comprising the nucleic acid molecules described further comprises a pharmaceutically acceptable carrier. In one embodiment, the pharmaceutically acceptable carrier does not include tissue or cell culture media.

### Administration & Efficacy

As used herein, the terms “administering,” “introducing” and “transplanting” are used interchangeably in the context of the placement of cells, e.g. hematopoietic progenitor cells, as described herein into a subject, by a method or route which results in at least partial localization of the introduced cells at a desired site, such as a site of injury or repair, such that a desired effect(s) is produced. The cells e.g. hematopoietic progenitor cells, or their differentiated progeny can be administered by any appropriate route which results in delivery to a desired location in the subject where at least a portion of the implanted cells or components of the cells remain viable. The period of viability of the cells after administration to a subject can be as short as a few hours, e.g., twenty-four hours, to a few days, to as long as several years, i.e., long-term engraftment. For example, in some embodiments of the aspects described herein, an effective amount of hematopoietic progenitor cells or engineered cells with reduced ELANE expression is administered via a systemic route of administration, such as an intraperitoneal or intravenous route.

When provided prophylactically, hematopoietic progenitor cells or engineered cells with reduced ELANE expression described herein can be administered to a subject in advance of any symptom of a SCN. Accordingly, the prophylactic administration of a hematopoietic progenitor cell population serves to prevent a SCN, as disclosed herein.

When provided therapeutically, hematopoietic progenitor cells are provided at (or after) the onset of a symptom or indication of SCN, e.g., fevers, acute inflammation of the lungs (e.g., pneumonia), ear infections, and/or inflammation of tissues, such as the gums (gingivitis) or the delicate mucous membranes that line the mouth (stomatitis).

In some embodiments of the aspects described herein, the hematopoietic progenitor cell population or engineered cells with reduced ELANE expression being administered according to the methods described herein comprises allogeneic hematopoietic progenitor cells obtained from one or more donors. As used herein, “allogeneic” refers to a hematopoietic progenitor cell or biological samples comprising hematopoietic progenitor cells obtained from one or more different donors of the same species, where the genes at one or more loci are not identical. For example, a hematopoietic progenitor cell population or engineered cells with reduced ELANE expression being administered to a subject can be derived from umbilical cord blood obtained from one more unrelated donor subjects, or from one or more non-identical siblings. In some embodiments, syngeneic hematopoietic progenitor cell populations can be used, such as those obtained from genetically identical animals, or from identical twins. In other embodiments of this aspect, the hematopoietic progenitor cells are autologous cells; that is, the hematopoietic progenitor cells are obtained or isolated from a subject and administered to the same subject, i.e., the donor and recipient are the same.

For use in the various aspects described herein. an effective amount of hematopoietic progenitor cells or engineered cells with reduced ELANE expression, comprises at least 10² cells, at least 5×10² cells, at least 10³ cells, at least 5×10³ cells, at least 10⁴ cells, at least 5×10⁴ cells, at least 10⁵ cells, at least 2×10⁵ cells, at least 3×10⁵ cells, at least 4×10⁵ cells, at least 5×10⁵ cells, at least 6×10⁵ hematopoietic progenitor cells, at least 7×10⁵ cells, at least 8×10⁵ cells, at least 9×10⁵ cells, at least 1×10⁶ cells, at least 2×10⁶ cells, at least 3×10⁶ cells, at least 4×10⁶ cells, at least 5×10⁶ cells, at least 6×10⁶ cells, at least 7×10⁶ cells, at least 8×10⁶ cells, at least 9×10⁶ cells, or multiples thereof. The hematopoietic progenitor cells or engineered cells with reduced ELANE expression can be derived from one or more donors, or can be obtained from an autologous source. In some embodiments of the aspects described herein, the hematopoietic progenitor cells are expanded in culture prior to administration to a subject in need thereof.

In one embodiment, the term “effective amount” as used herein refers to the amount of a population of human hematopoietic progenitor cells or their progeny needed to alleviate at least one or more symptom of a neutropenia, e.g., Cyclic neutropenia or SCN, and relates to a sufficient amount of a composition to provide the desired effect, e.g., treat a subject having a SCN. The term “therapeutically effective amount” therefore refers to an amount of hematopoietic progenitor cells, or genetic engineered cells described herein or their progeny or a composition comprising hematopoietic progenitor cells, or genetic engineered cells described herein or their progeny that is sufficient to promote a particular effect when administered to a typical subject, such as one who has or is at risk for SCN. An effective amount as used herein would also include an amount sufficient to prevent or delay the development of a symptom of the disease, alter the course of a symptom disease (for example but not limited to, slow the progression of a symptom of the disease), or reverse a symptom of the disease. It is understood that for any given case, an appropriate “effective amount” can be determined by one of ordinary skill in the art using routine experimentation.

As used herein, “administered” refers to the delivery of a hematopoietic stem cell composition as described herein into a subject by a method or route which results in at least partial localization of the cell composition at a desired site. A cell composition can be administered by any appropriate route which results in effective treatment in the subject, i.e. administration results in delivery to a desired location in the subject where at least a portion of the composition delivered, i.e. at least 1×10⁴ cells are delivered to the desired site for a period of time. Modes of administration include injection, infusion, instillation, or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. For the delivery of cells, administration by injection or infusion is generally preferred.

In one embodiment, the cells as described herein are administered systemically. The phrases “systemic administration,” “administered systemically”, “peripheral administration” and “administered peripherally” as used herein refer to the administration of a population of hematopoietic progenitor cells other than directly into a target site, tissue, or organ, such that it enters, instead, the subject's circulatory system and, thus, is subject to metabolism and other like processes.

The efficacy of a treatment comprising a composition as described herein for the treatment of a SCN can be determined by the skilled clinician. However, a treatment is considered “effective treatment,” as the term is used herein, if any one or all of the signs or symptoms of, e.g., fevers, acute inflammation of the lungs (e.g., pneumonia), ear infections, and/or inflammation of tissues, such as the gums (gingivitis) or the delicate mucous membranes that line the mouth (stomatitis), or other clinically accepted symptoms or markers of disease are improved or ameliorated, e.g., by at least 10% following treatment with any reagent, composition, or cell described herein. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease; or (2) relieving the disease, e.g., causing regression of symptoms; and (3) preventing or reducing the likelihood of the development of a future onset of the disease.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

The disclosure described herein, in a preferred embodiment, does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Furthermore, the disclosure described herein does not concern the destruction of a human embryo.

This invention is further illustrated by the following example which should not be construed as limiting. The contents of all references cited throughout this application, as well as the figures and table are incorporated herein by reference.

Some embodiments of the invention described herein can be defined according to any of the following numbered paragraphs:

1. A modified synthetic nucleic acid comprising a nucleic acid sequence shown in Table 1, SEQ ID NOS: 1-374, wherein there is at least one chemical modification to a nucleotide in the nucleic acid molecule.

2. The modified synthetic nucleic acid of any preceding paragraph, wherein the at least one chemical modifications is located at one or more terminal nucleotides in nucleic acid molecule.

3. The modified synthetic nucleic acid of any preceding paragraph, wherein the at least one chemical modification is selected from the group consisting of 2′-O-methyl 3′phosphorothioate (MS), 2′-O-methyl-3′-phosphonoacetate (MP), 2′-0-Ci-4alkyl, 2′-H, 2′-O-Ci.3alkyl-O-Ci.3alkyl, 2′-F, 2′-NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag).

4. The modified synthetic nucleic acid of any preceding paragraph, wherein the at least one chemical modification is located only at the 3′ end, or added only at the 5′ end, or added at both the 5′ and 3′ ends of the synthetic nucleic acid molecule.

5. The modified synthetic nucleic acid of any preceding paragraph wherein the at least one chemical modification is located to first three nucleotides and to the last three nucleotides of the synthetic nucleic acid molecule.

6. The modified synthetic nucleic acid of any preceding paragraph, wherein the nucleic acid sequence further comprising a crRNA/tracrRNA sequence.

7. The modified synthetic nucleic acid of any preceding paragraph, wherein the nucleic acid molecule is a single guide RNA (sgRNA).

8. The modified synthetic nucleic acid of any preceding paragraph, for use in the ex vivo targeted genome editing of the ELANE gene in a progenitor cell purpose.

9. The modified synthetic nucleic acid of any preceding paragraph, for use in an ex vivo method of producing a progenitor cell or a population of progenitor cells wherein the cells or the differentiated progeny therefrom have decreased ELANE mRNA or protein expression.

10. The modified synthetic nucleic acid of any preceding paragraph, for use in an ex vivo method of producing an isolated genetic engineered human cell or a population of genetic engineered human cells having at least one genetic modification.

11. The modified synthetic nucleic acid of any preceding paragraph, wherein the modified synthetic nucleic acid molecule is used in combination with a DNA-targeting endonuclease Cas (CRISPR-associated) protein in a ribonucleoprotein (RNP) complex.

12. The modified synthetic nucleic acid of any preceding paragraph, wherein the Cas protein is Cas 9.

13. The modified synthetic nucleic acid of any preceding paragraph, wherein the RNP complex is used in the electroporation of cells.

14. The modified synthetic nucleic acid of any preceding paragraph, wherein the step of electroporation is performed in a solution comprising glycerol.

15. The modified synthetic nucleic acid of any preceding paragraph, wherein the isolated human cell is a hematopoietic progenitor cell or a hematopoietic stem cell.

16. The modified synthetic nucleic acid of any preceding paragraph, wherein the hematopoietic progenitor is a cell of the erythroid lineage.

17. The modified synthetic nucleic acid of any preceding paragraph, wherein the isolated human cell is an induced pluripotent stem cell.

18. The modified synthetic nucleic acid of any preceding paragraph, wherein the progenitor cell or ascquires at least one genetic modification.

19. The modified synthetic nucleic acid of any preceding paragraph, wherein the at least one genetic modification is a deletion, insertion or substitution of the genetic sequence of the cell.

20. A composition comprising a modified synthetic nucleic acid molecule of any preceding paragraph.

21. The composition of any preceding paragraph, further comprising a DNA-targeting endonuclease Cas (CRISPR-associated) protein.

22. The composition of any preceding paragraph, wherein the Cas protein is Cas 9.

23. The composition of any preceding paragraph for use in an ex vivo method of producing a progenitor cell or a population of progenitor cells wherein the cells or the differentiated progeny therefrom have decreased ELANE mRNA or protein expression.

24. The composition of any preceding paragraph for use in an ex vivo method of producing an isolated genetic engineered human cell or a population of progenitor cells having at least one genetic modification.

25. The composition of any preceding paragraph, wherein the composition is used in the electroporation of cells.

26. The composition of any preceding paragraph, wherein the step of electroporation is performed in a solution comprising glycerol.

27. A ribonucleoprotein (RNP) complex comprising a DNA-targeting endonuclease Cas (CRISPR-associated) protein and a modified synthetic nucleic acid molecule of any preceding paragraph.

28. The RNP complex of any preceding paragraph for use in an ex vivo method of producing a progenitor cell or a population of progenitor cell wherein the cells or the differentiated progeny thereof have decreased ELANE mRNA or protein expression.

29. The RNP complex of any preceding paragraph for use in an ex vivo method of producing an isolated genetic engineered human cell or a population of genetic engineered human cells having at least one genetic modification.

30. The RNP complex of any preceding paragraph, wherein the RNP complex is used in the electroporation of cells.

31. The RNP complex of any preceding paragraph, wherein wherein the step of electroporation is performed in a solution comprising glycerol.

32. A method for producing a progenitor cell or a population of progenitor cells having decreased ELANE mRNA or protein expression, the method comprising contacting an isolated progenitor cell with an effective amount of a modified synthetic nucleic acid of any preceding paragraph, a composition of any preceding paragraph, or a ribonucleoprotein (RNP) complex of any preceding paragraph, whereby the contacted cells or the differentiated progeny cells therefrom have decreased ELANE mRNA or protein expression.

33. The method of any preceding paragraph, wherein the at least one genetic modification is a deletion, insertion or substitution of the genetic sequence of the cell.

34. The method of any preceding paragraph, wherein the isolated progenitor cell is a hematopoietic progenitor cell or a hematopoietic stem cell.

35. The method of any preceding paragraph, wherein the hematopoietic progenitor is a cell of the erythroid lineage.

36. The method of any preceding paragraph, wherein the isolated progenitor cell is an induced pluripotent stem cell.

37. The method of any preceding paragraph, wherein the isolated progenitor cell is contacted ex vivo or in vitro.

38. The method of any preceding paragraph, wherein the contacted progenitor cell acquires at least one genetic modification.

39. The method of any preceding paragraph, wherein the contacted progenitor cell or contacted cell are further electroporated.

40. The method of any preceding paragraph, wherein the step of electroporation is performed in a solution comprising glycerol.

41. A population of genetically edited human cells having at least one genetic modification resulting in decreased ELANE mRNA or protein expression.

42. The population of any preceding paragraph, wherein the genetically edited human cells are isolated.

43. A population of genetically edited progenitor cells having at least one genetic modification resulting in decreased ELANE mRNA or protein expression.

44. A composition comprising isolated genetically edited human cells of any preceding paragraph.

45. A composition comprising genetically edited progenitor cells of any preceding paragraph.

46. A method of treating a disease associated with abnormal ELANE gene expression, the method comprising, administering to a subject in need thereof a gene editing agent that targets the ELANE gene, wherein the edited ELANE gene comprises at least one mutation selected from the group consisting of a loss-of-function mutation, a through stop-gain mutation, a frameshift mutation, a premature termination codon, and a splicing mutation that encodes a premature termination codon.

47. The method of any preceding paragraph, wherein the disease is selected from the group consisting of chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis, chronic kidney disease, cyclic neutropenia, and severe congential neutropenia.

48. The method of any preceding paragraph, wherein the gene editing agent is selected from the group consisting of clustered regularly interspaced short palindromic repeats (CRISPR), Transcription activator-like effector nucleases (TALEN), Meganuclease, Zinc finger nucleases, Homologous recombination, and deaminase fusion proteins.

49. The method of any preceding paragraph, wherein the at least one mutation results in induction of nonsense-mediated decay, translational repression, or knockdown of transcript.

50. The method of any preceding paragraph, wherein the abnormal ELANE gene expression results in abnormal neutrophil elastase protein.

51. The method of claim 46, wherein the gene editing agent is a modified synthetic nucleic acid of any preceding paragraph, a composition of any preceding paragraph, or a ribonucleoprotein (RNP) complex of any preceding paragraph.

### EXAMPLES

### Therapeutic Gene Editing for Elane-Associated Severe Congenital Neutropenia

Knockout of ELANE with NMD induction might bypass dominantly acting missense or NMD-escape frameshift mutations. By preventing production of abnormal ELANE, wild-type ELANE from the non-mutant allele could continue to provide normal neutrophil elastase function. Even biallelic loss-of-function mutations might be tolerable. Although neutrophil elastase is a major serine protease, neutrophils have other serine proteases such as proteinase 3 and cathespin G that might partially replace the function in certain contexts. For example, in a mouse model, ELANE deficiency is dispensable for response to Aspergillus pneumonia whereas neutrophil oxidative function is essential for survival1. The Papillon-Lefevre syndrome results from deficiency of cathespin C (dipeptidyl peptidase I, DPPI, CTSC). The consequence is absence of the major neutrophil secondary granule serine proteases, including neutrophil elastase, cathespin G, proteinase 3, and neutrophil serine protease 42. Although patients suffer from periodontitis, they do not have marked immunodeficiency. The relatively mild phenotype of combined neutrophil serine protease deficiency suggests that isolated deficiency of ELANE would be unlikely to result in major infectious risk.

Severe congenital neutropenia (SCN) is a life-threatening disorder caused by dominant mutations of ELANE (neutrophil elastase) that interfere with normal neutrophil maturation. Despite ameliorating G-CSF therapy, patients remain at elevated lifelong risk of myeloid leukemia. Recently the inventors developed a protocol for highly efficient gene editing of human hematopoietic stem and progenitor cells (HSPCs) by electroporation of Cas9 and sgRNA ribonucleoprotein (RNP). Here the inventors show by genome editing HSPCs that ELANE frameshift alleles that mimic SCN-associated mutations escape nonsense-mediated decay (NMD) and result in neutrophil maturation arrest. Conversely the inventors demonstrate that highly efficient ELANE gene editing of upstream exons elicits NMD and avoids neutrophil maturation arrest. Upstream exon ELANE gene editing overcomes neutrophil maturation arrest in HSPCs engineered to have SCN-associated ELANE mutations as well as primary HSPCs from ELANE-mutant SCN patients. This autologous HSC editing approach represents a universal therapeutic gene editing strategy for ELANE mutant neutropenia.

CRISPR/Cas9 gene editing systems enable programmable targeting of the Cas9 endonuclease to specific genomic targets. This modality promises to enable genetic modification of autologous HSCs for amelioration of blood disorders. Although templated homology repair can directly correct disease-associated mutations in principle, challenges include the requirement for additional delivery of extrachromosomal DNA template, need for bespoke correction strategies for sundry patient-specific mutations, concurrent nonhomologous repair, and relative resistance of quiescent HSCs to homologous repair. Nonhomologous end joining would be a simpler and more efficient repair outcome but depends on identification of a suitable therapeutic target for gene disruption. For example, mere knockout of CCR5 coreceptor may prevent HIV infection³ and mere disruption of the BCL11A erythroid enhancer could result in therapeutic fetal hemoglobin induction⁴. Here we investigate the hypothesis that production of Cas9-mediated NMD-triggering ELANE frameshift alleles could be a universal therapeutic gene editing strategy to relieve the neutrophil maturation arrest caused by SCN-associated ELANE mutations.

### Methods

Preparation of 3xNLS-SpCas9.

The plasmid expressing 3xNLS-SpCas9 was constructed in the pET21a expression plasmid (Novagen). The recombinant S. pyogenes Cas9 with a 6xHis tag and c-Myc-like nuclear localization signal (NLS) at the N-terminus55, SV40 and nucleoplasmin NLS at the C-terminus was expressed in E. coli Rosetta (DE3)pLysS cells (EMD Millipore). Cells were grown at 37 degrees to an OD600 of −0.2, then shifted to 18 degrees and induced at an OD600 of ˜0.4 for 16 hours with IPTG (1 mM final concentration). Following induction, cells were resuspended with Nickel-NTA buffer (20 mM TRIS+500 mM NaCl+20 mM imidazole+1 mM TCEP, pH 8.0) supplemented with HALT protease inhibitor and lysed with M-110s Microfluidizer (Microfluidics) following the manufacturer's instructions. The protein was purified with Ni-NTA resin and eluted with elution buffer (20 mM TRIS, 250 mM NaCl, 250 mM Imidazole, 10% glycerol, pH 8.0). Subsequently, 3xNLS-SpCas9 protein was further purified by cation exchange chromatography (Column=5 ml HiTrap-S, Buffer A=20 mM HEPES pH 7.5+1 mM TCEP, Buffer B=20 mM HEPES pH 7.5+1 M NaCl+1 mM TCEP, Flow rate=5 ml/min, CV=column volume=5 ml) and size-exclusion chromatography (SEC) on Hiload 16/600 Superdex 200 pg column (Isocratic size-exclusion running buffer=20 mM HEPES pH 7.5, 150 mM NaCl, 1 mM TCEP), then reconstituted in a formulation of 20 mM HEPES+150 mM NaCl, pH 7.4.

RNP Electroporation.

Electroporation was performed using Lonza 4D Nucleofector (V4XP-3032 for 20 μl Nucleocuvette Strips or V4XP-3024 for 100 μl Nucleocuvettes) as the manufacturer's instructions. The modified synthetic sgRNA (2′-O-methyl 3′ phosphorothioate modifications in the first and last 3 nucleotides) was from Synthego. sgRNA concentration is calculated using the full-length product reporting method, which is 3-fold lower than the OD reporting method. For 20 μl Nucleocuvette Strips, the RNP complex was prepared by mixing 3xNLS-SpCas9 (200 pmol) and sgRNA (200 pmol, full-length product reporting method) and incubating for 15 min at room temperature immediately before electroporation. 30% glycerol solution was added to Cas9 protein prior to addition of sgRNA. 50 K HSPCs resuspended in 20 μl P3 solution were mixed with RNP and transferred to a cuvette for electroporation with program EO-100. For 100 μl cuvette electroporation, the RNP complex was made by mixing 1000 pmol Cas9 and 1000 pmol sgRNA. 5M HSPCs were resuspended in 100 μl P3 solution for RNP electroporation as described above. The electroporated cells were resuspended with X-VIVO media with cytokines and changed into EDM 24 h later for in vitro differentiation.

In Vitro Neutrophil Differentiation from HSPCs.

In order to obtain mature neutrophils in vitro, we carried out a two-stage in vitro differentiaiton from HSPCs (also CD34+ cells). After thawing, CD34+ cells were cultured in StemSpan SFEM medium (StemCell Technologies, Vancouver, Canada) with 1x CD34+ cells expansion supplement (StemCell Technologies, Vancouver, Canada) for 2 days for recovery. To induce neutrophil differentiation, the CD34+ cells were cultured in StemSpan SFEM medium supplemented with the cytokine cocktail of 50 ng/mL SCF, 100 ng/mL Flt3L, 5 ng/mL IL-6, 5 ng/mL GM-CSF and 3 ng/mL G-CSF for 4 days (stage 1). The cells were further cultured for 6-7 days in the same medium without GM-CSF for neutrphil maturation (stage 2). For consecutive culture, the medium was changed every three days, and fresh cytokines were added. All these cytokines were of human origin and purchased from PeproTech (Rocky Hill, N.J.).

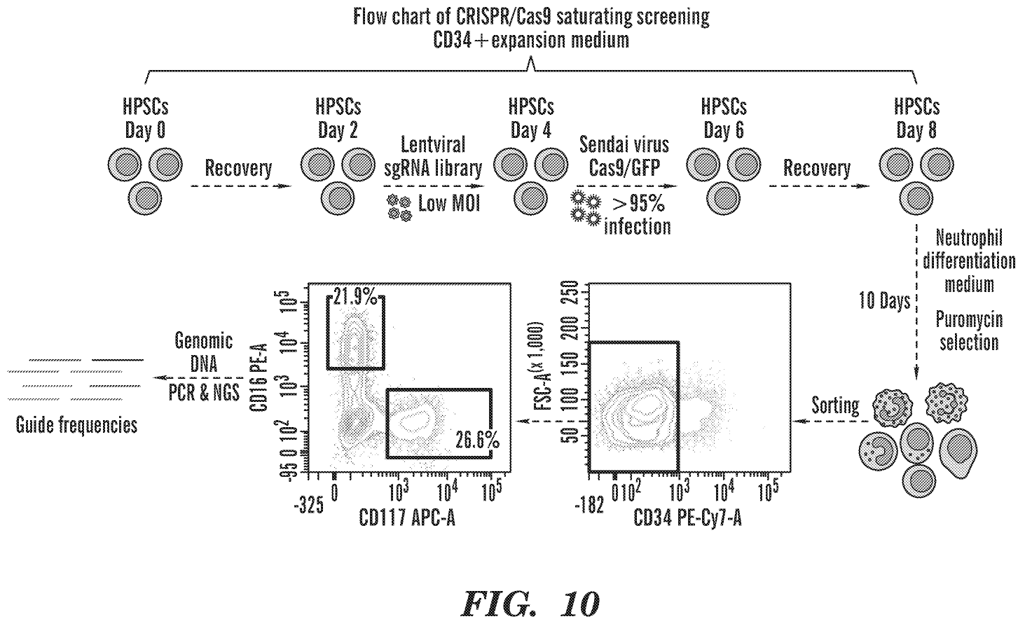

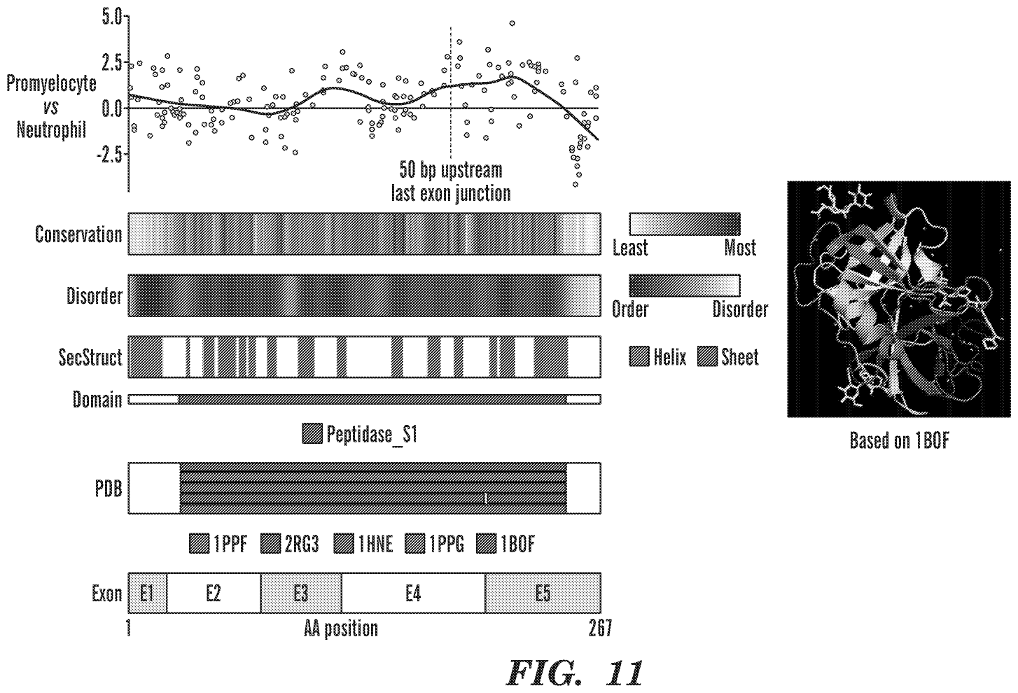

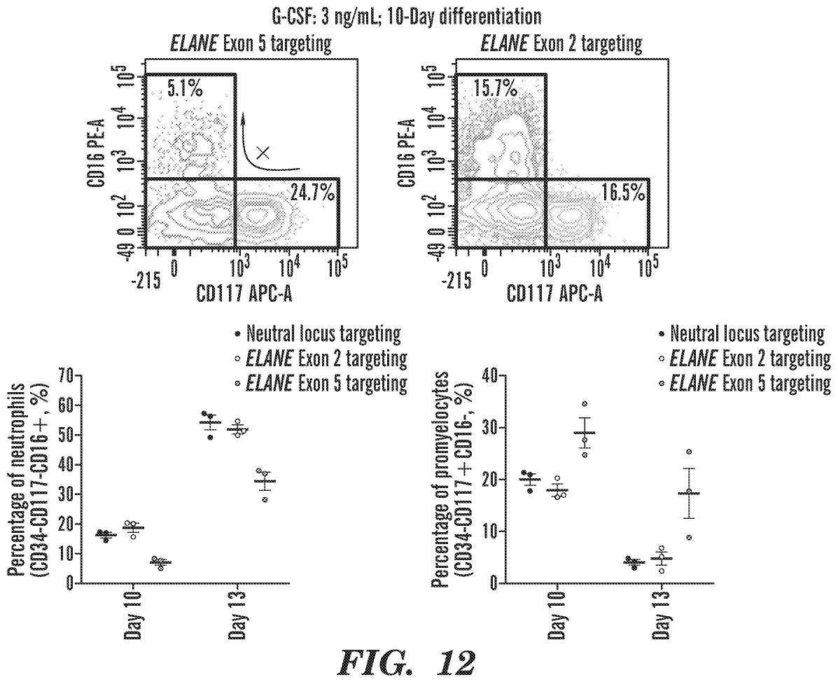

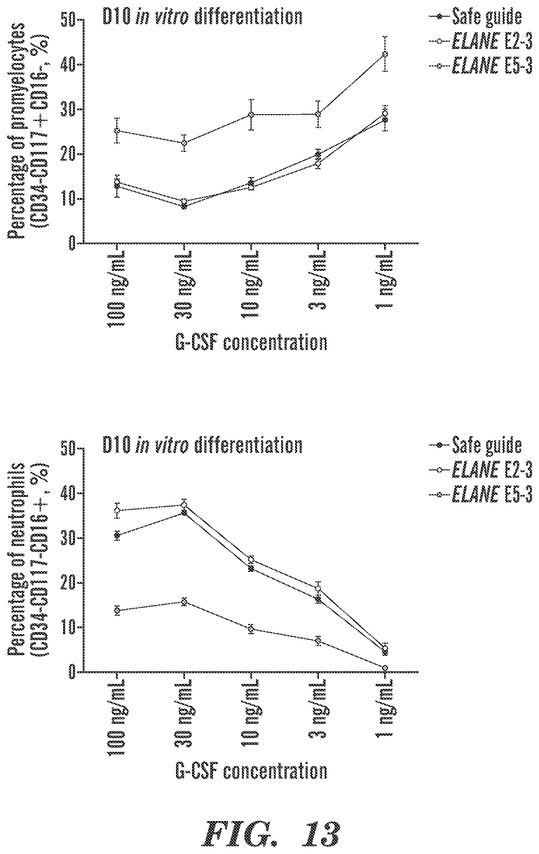

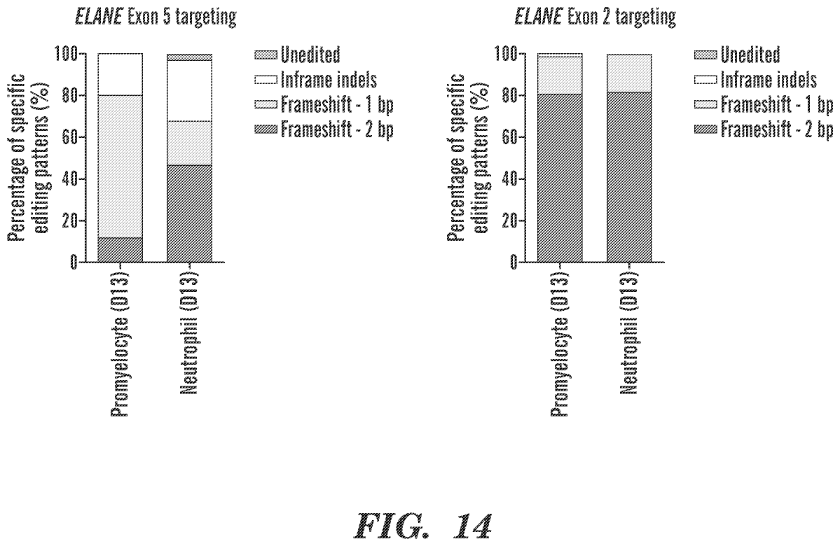

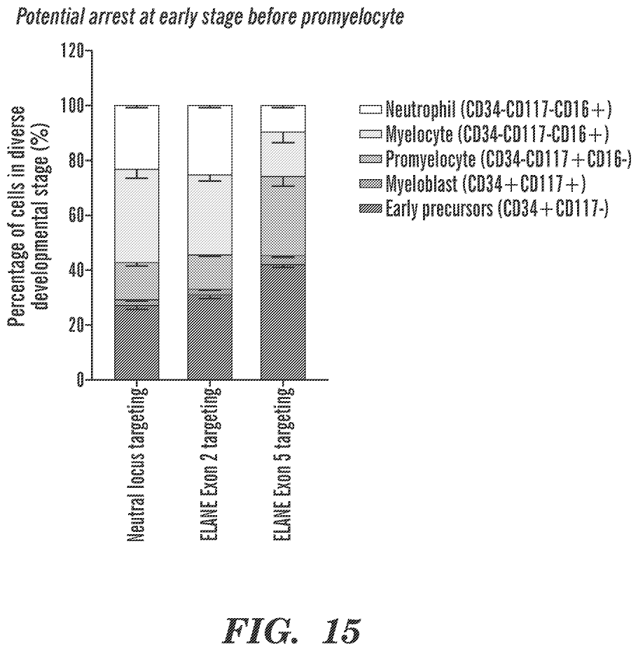

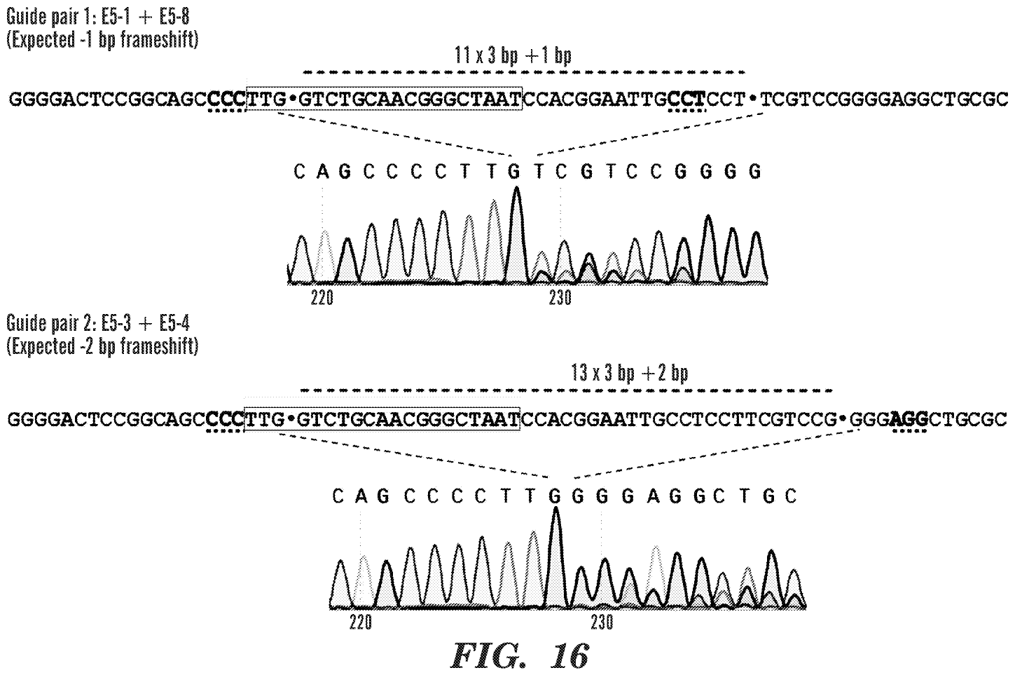

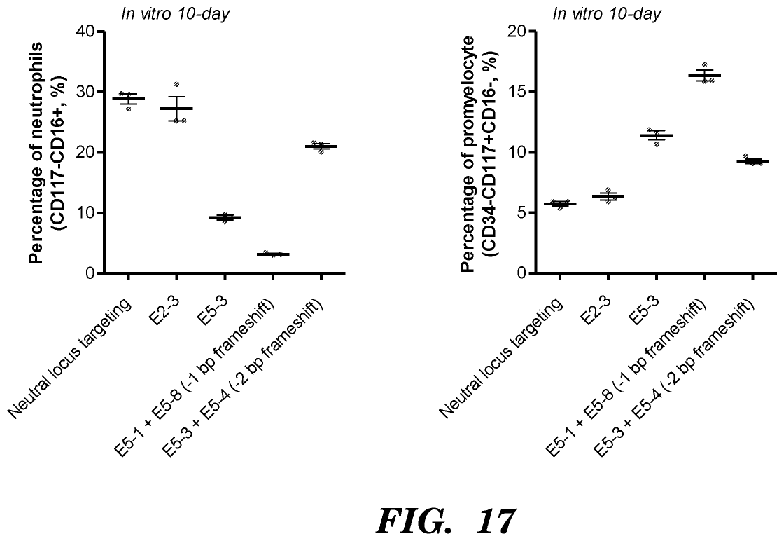

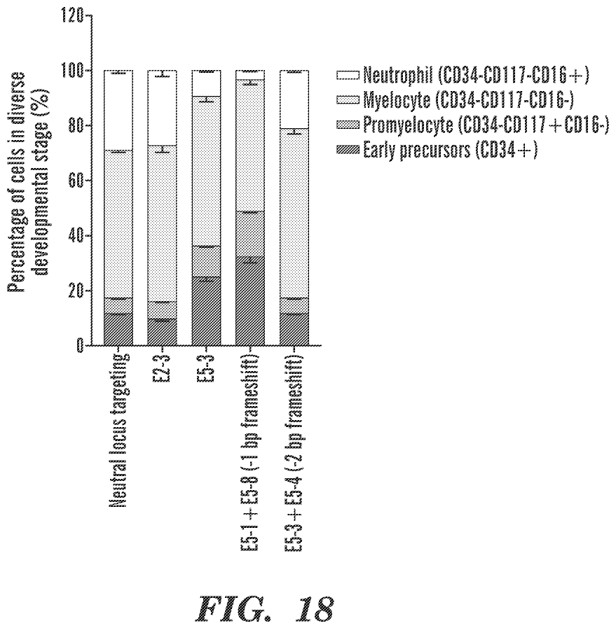

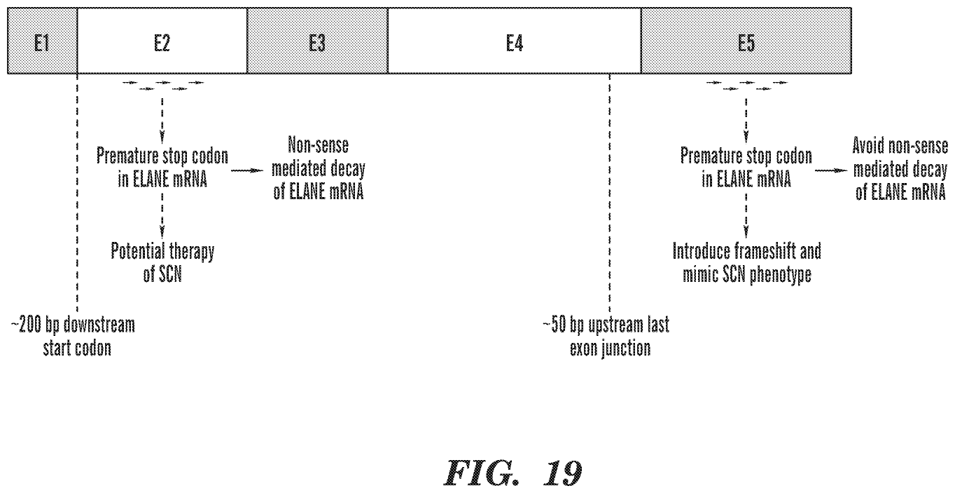

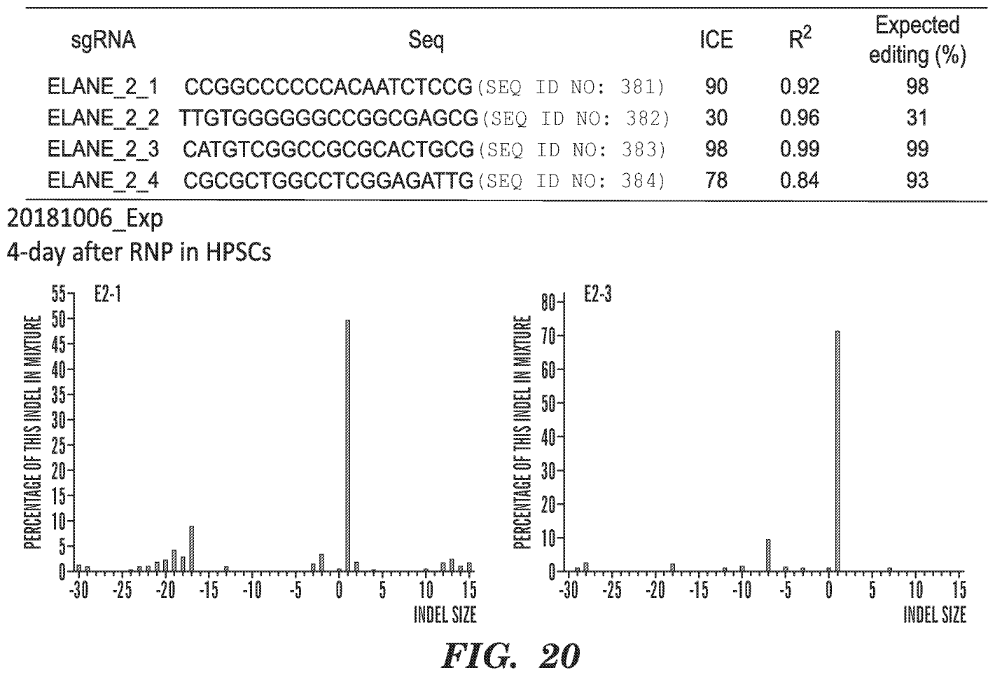

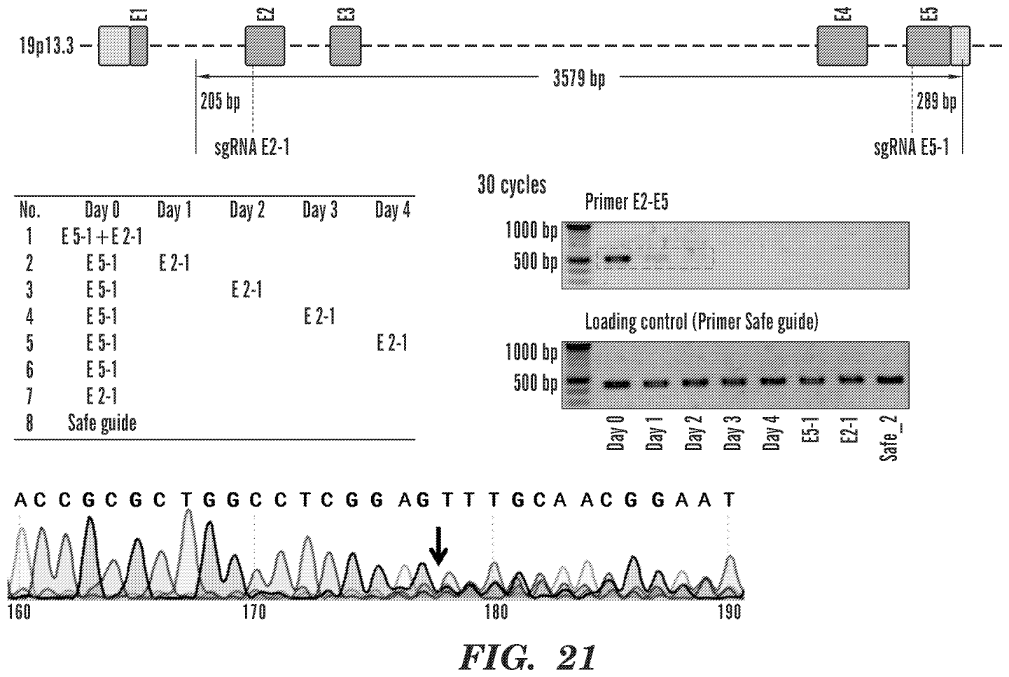

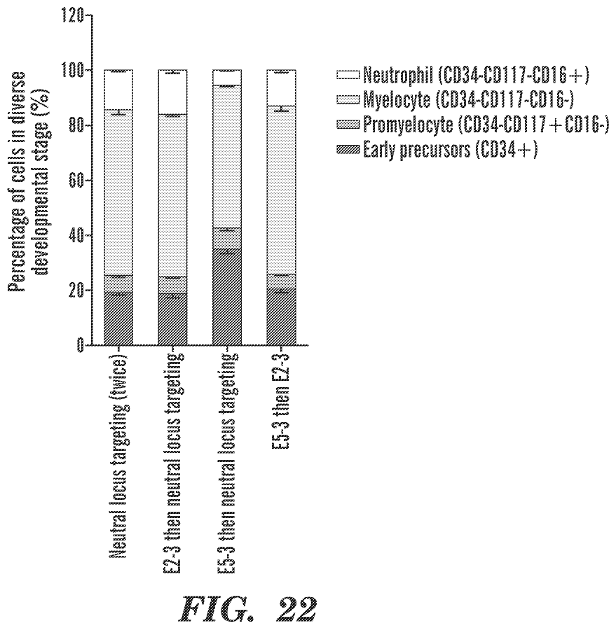

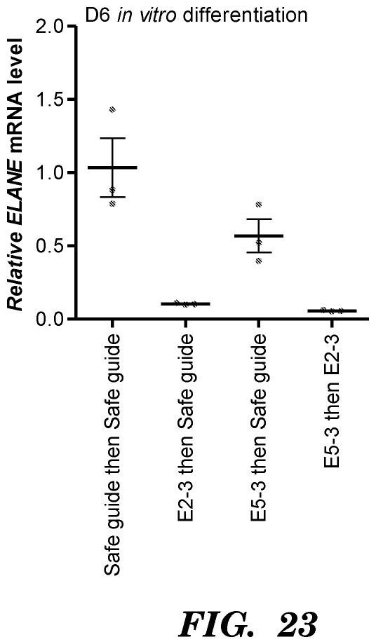

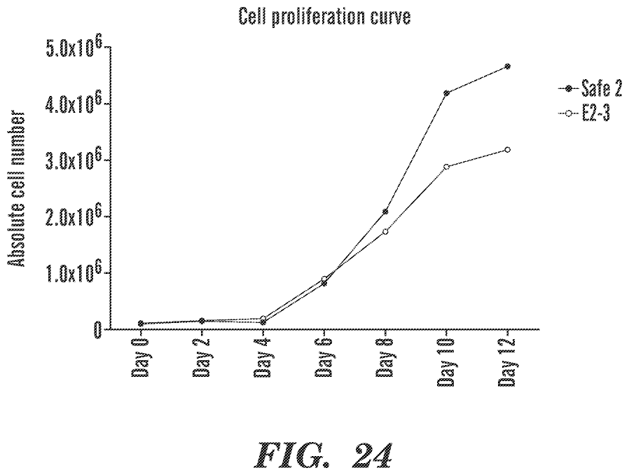

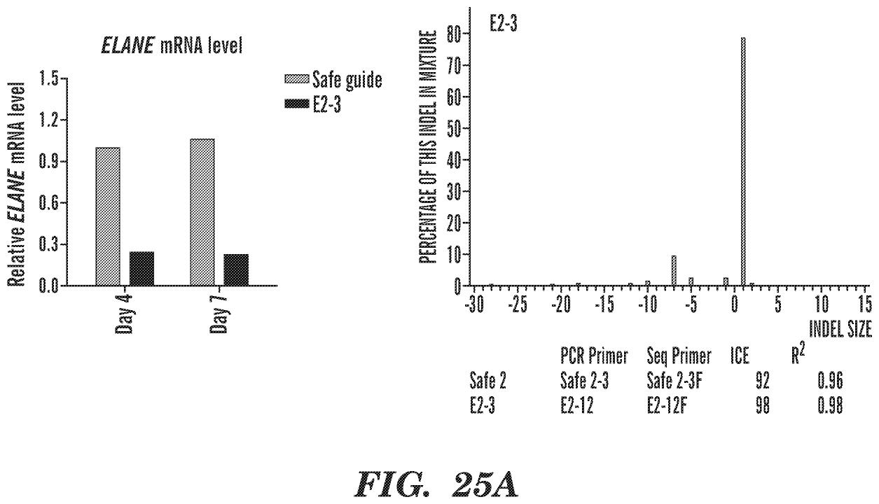

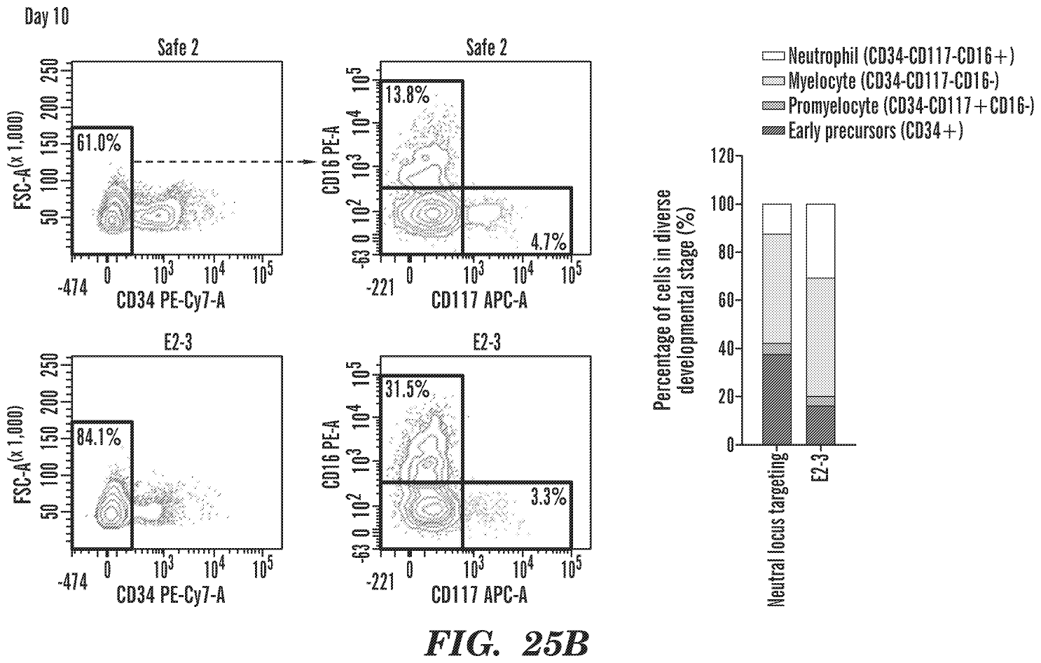

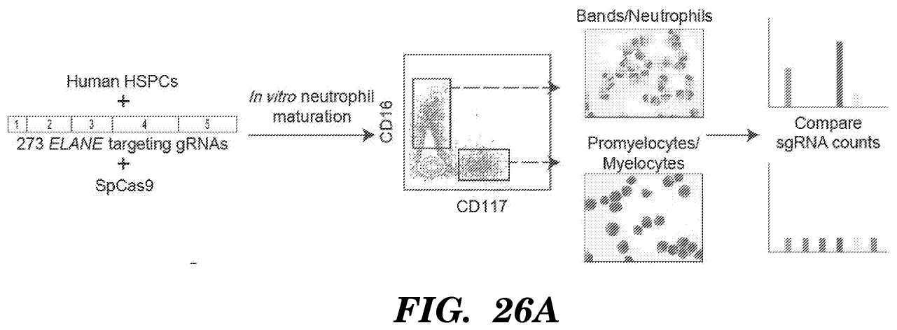

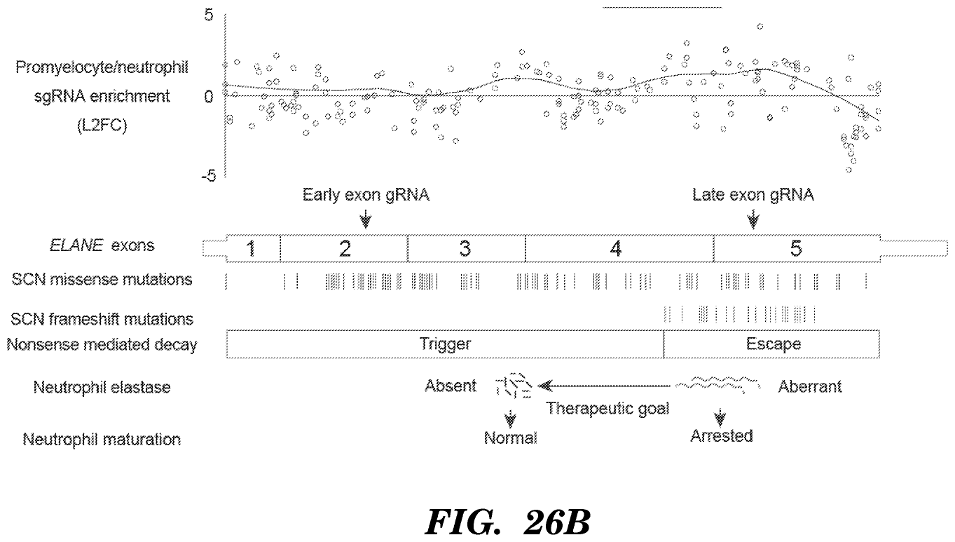

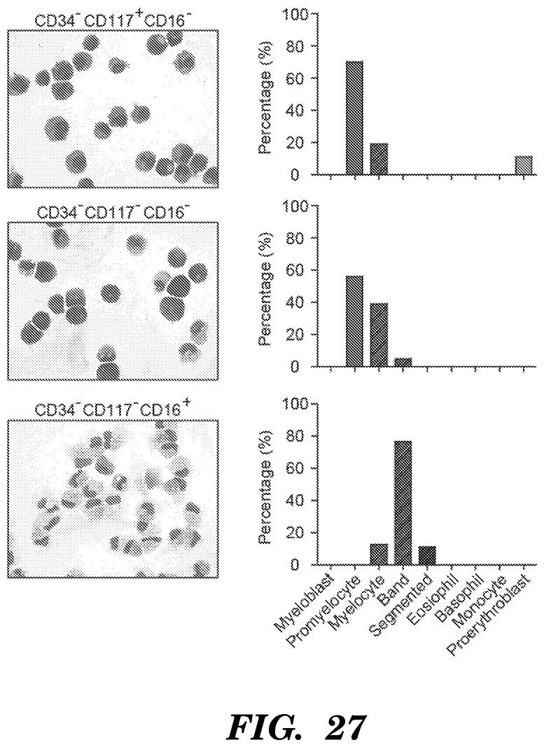

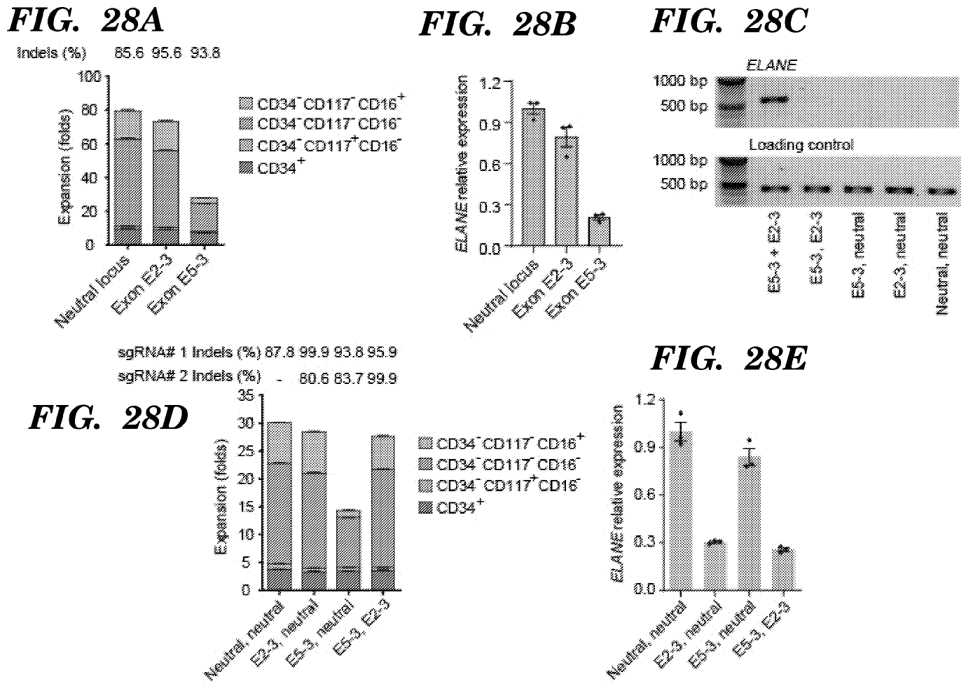

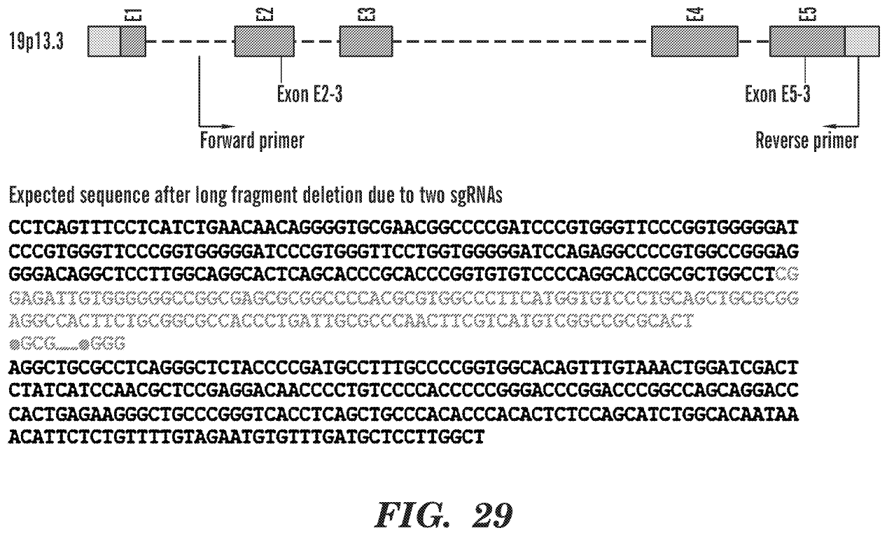

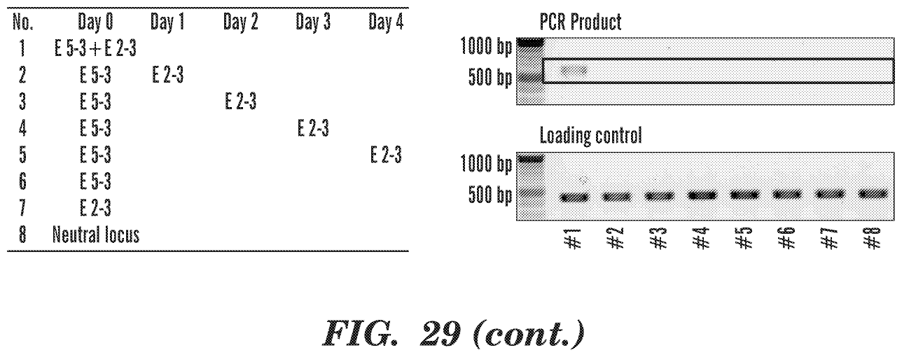

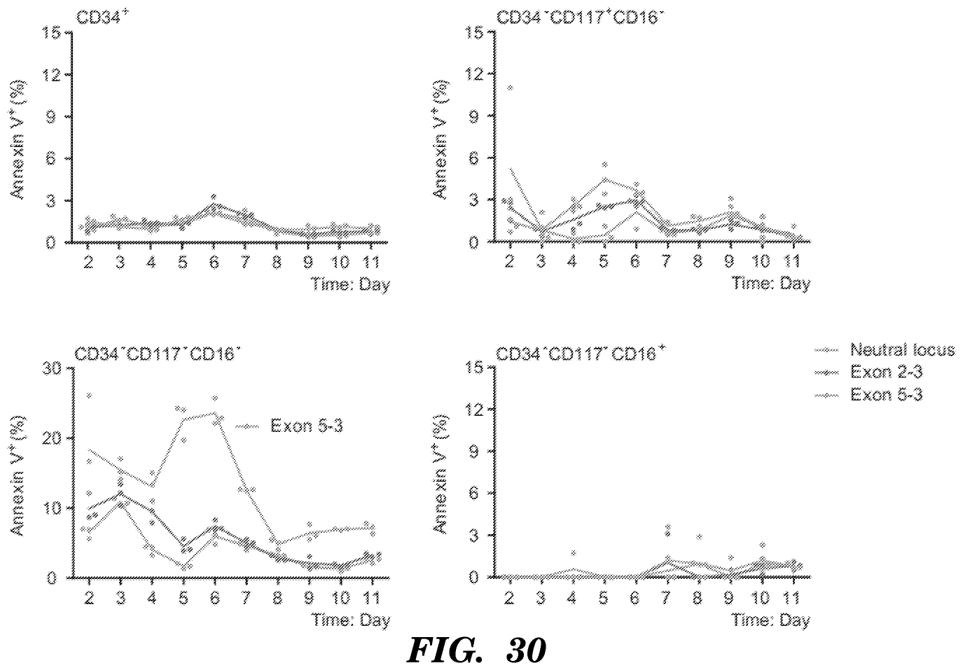

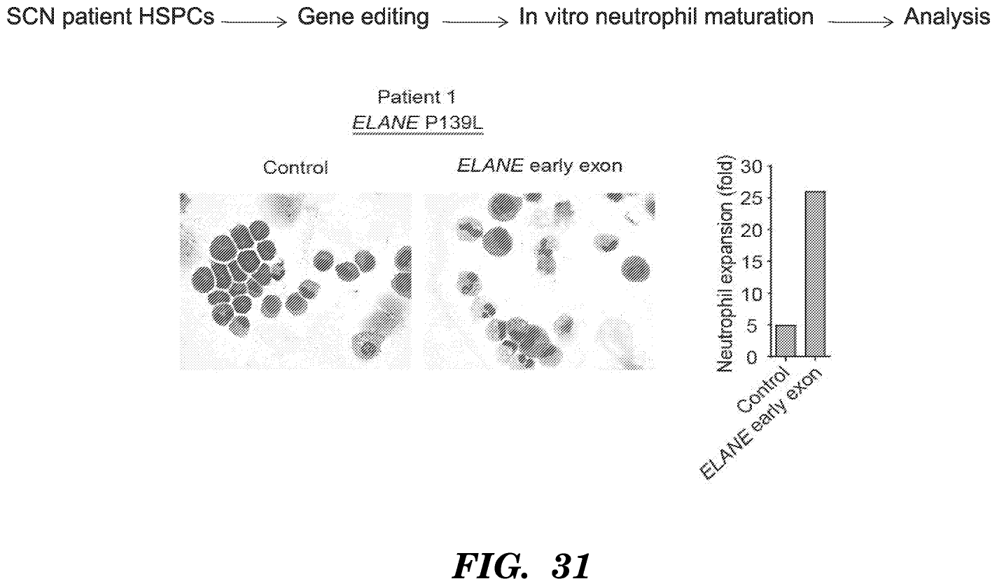

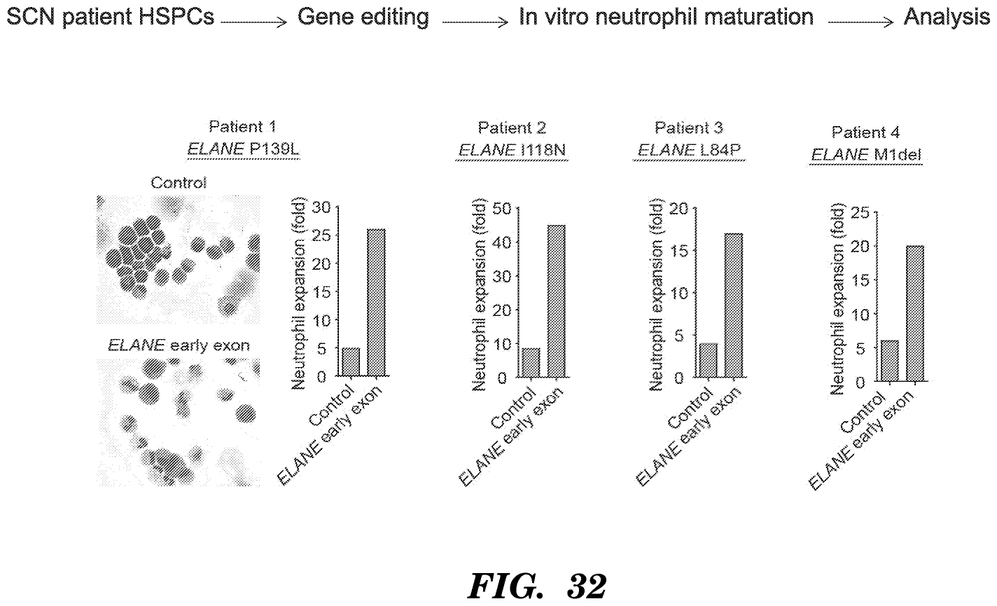

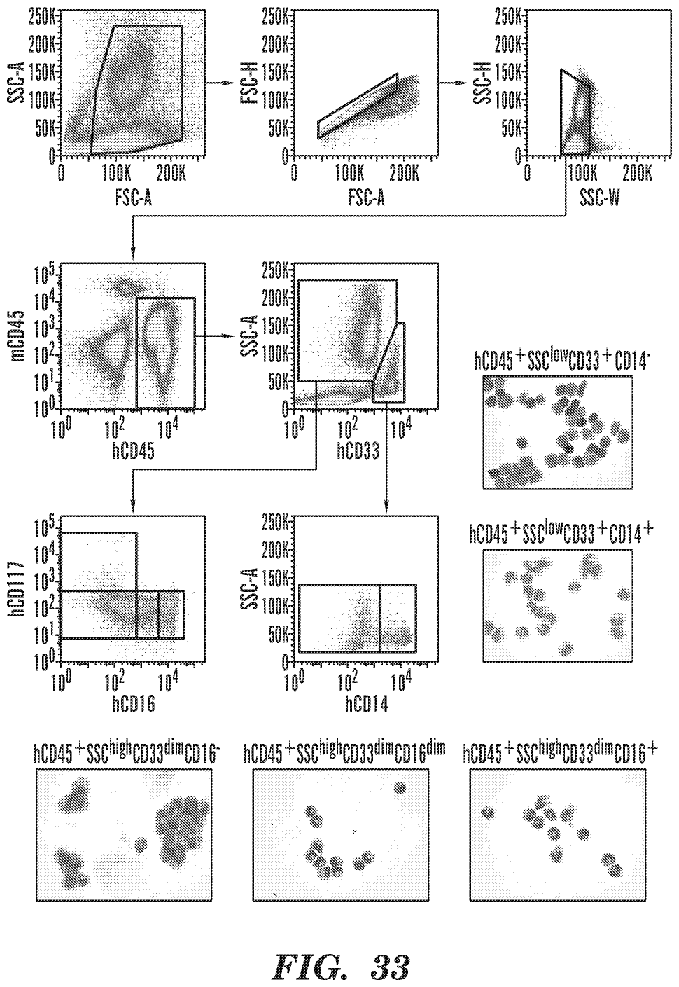

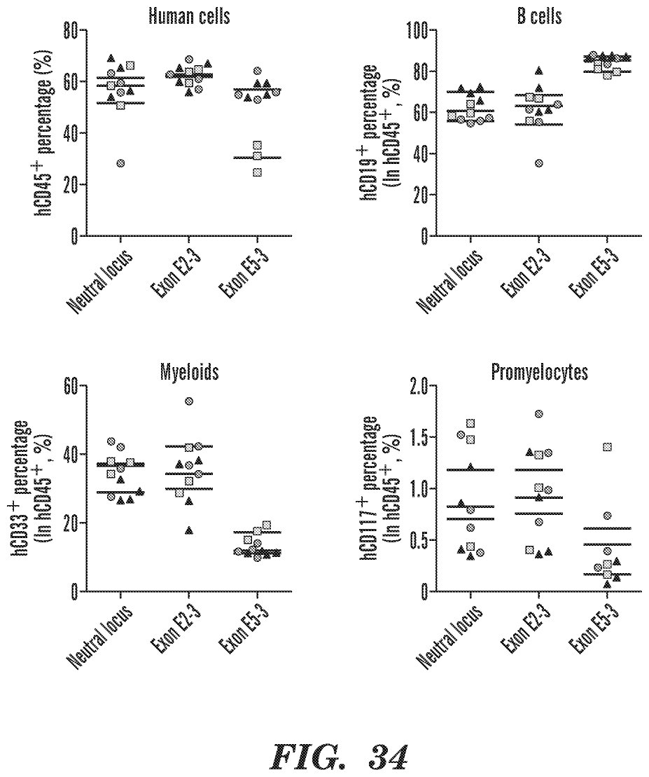

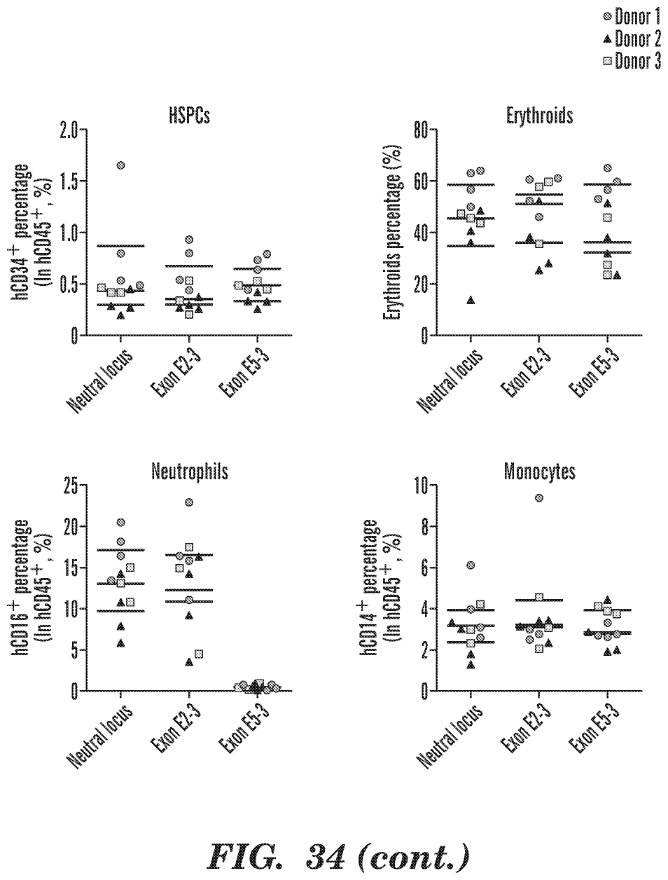

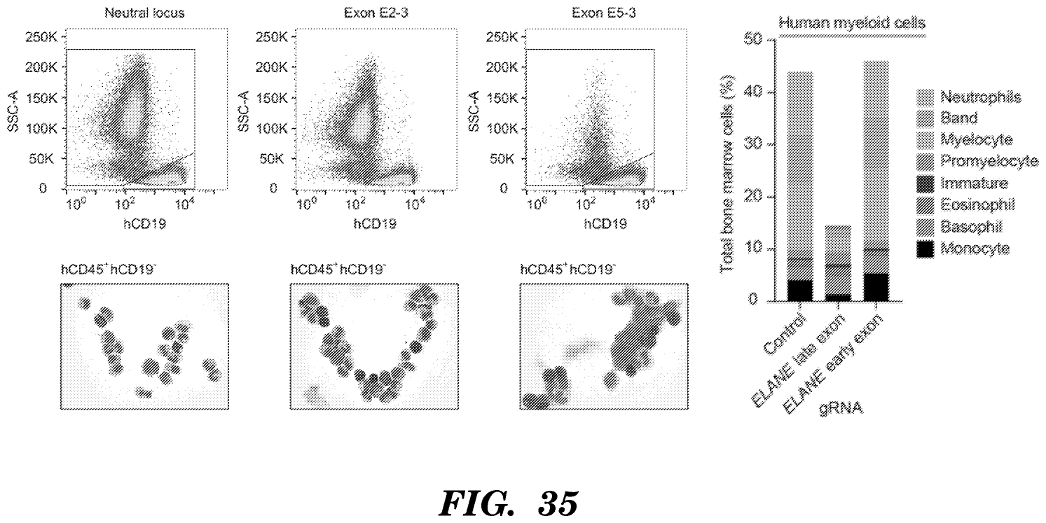

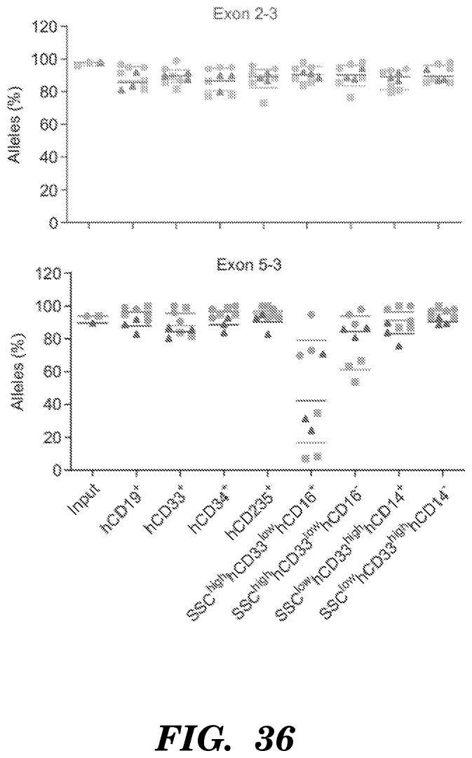

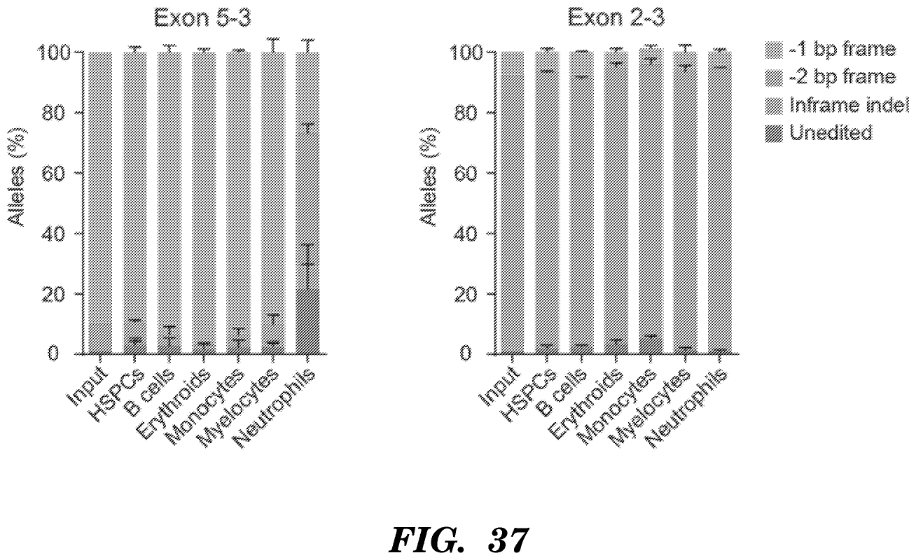

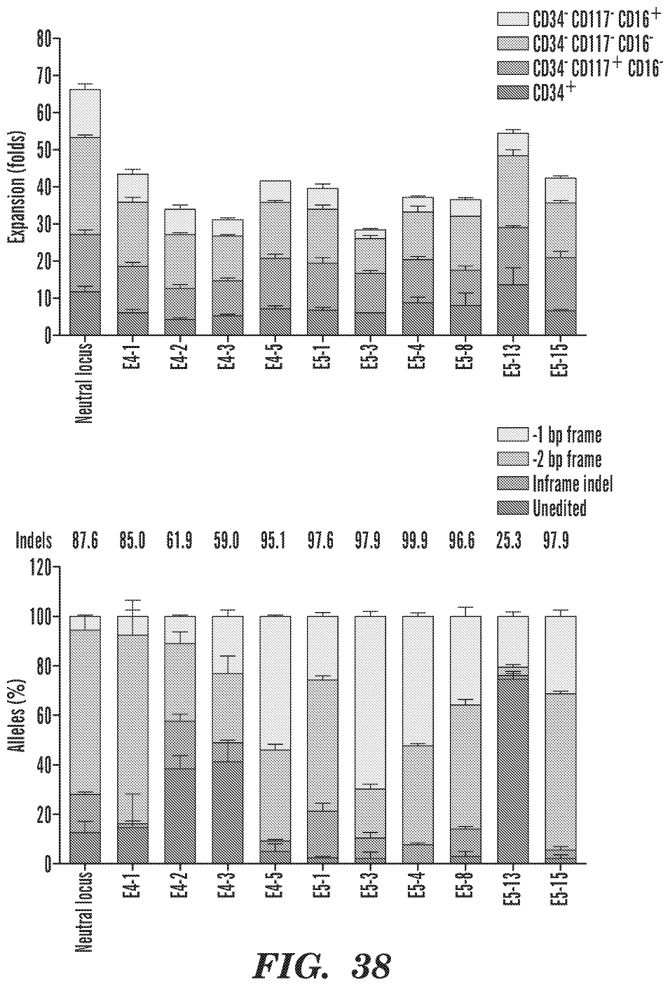

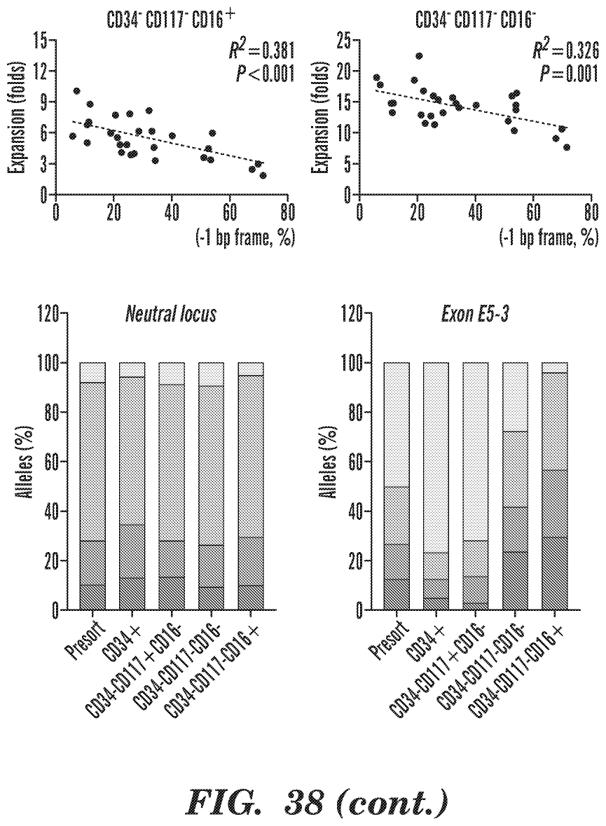

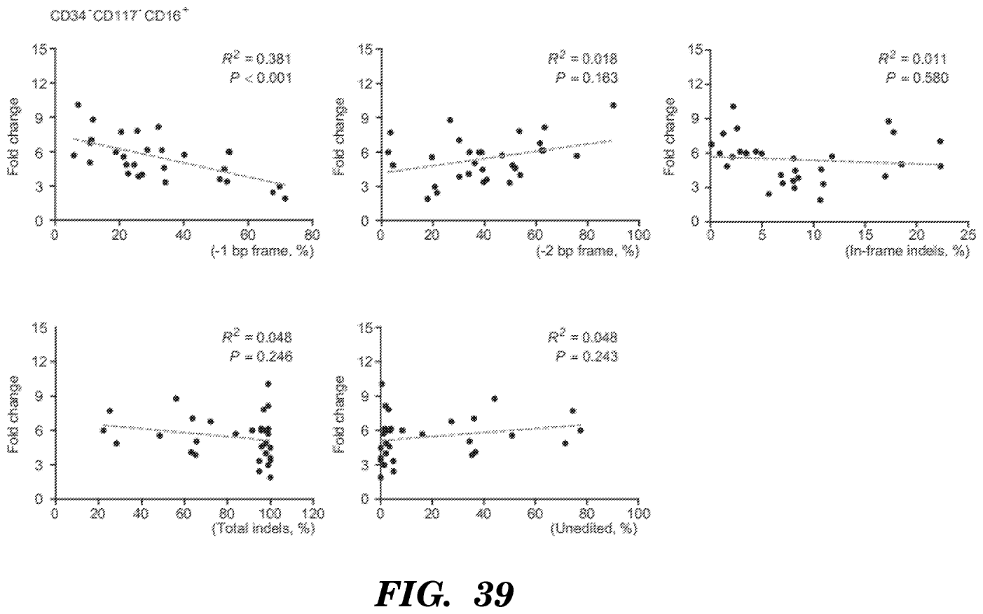

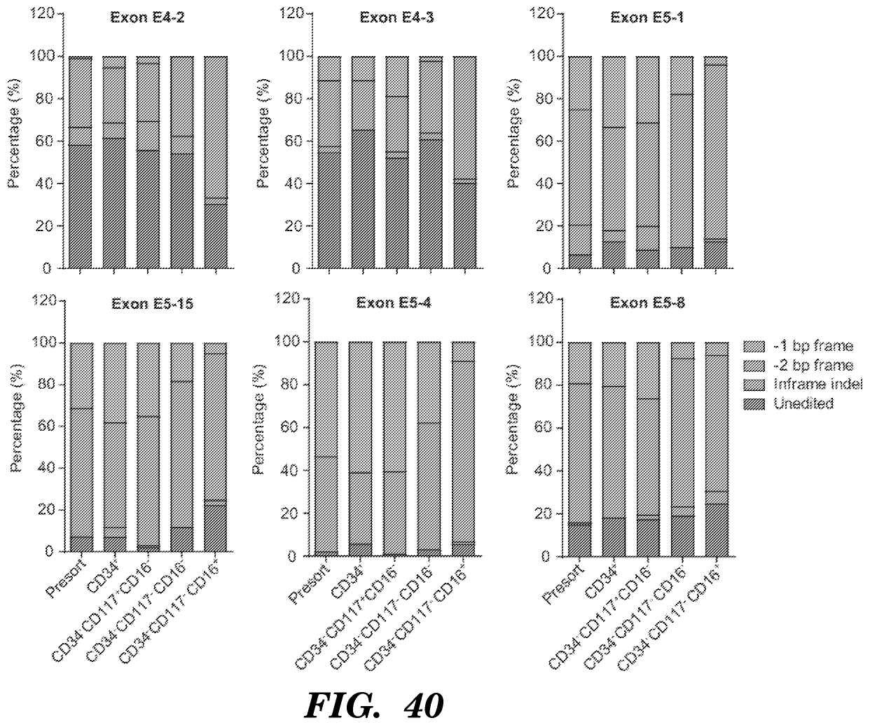

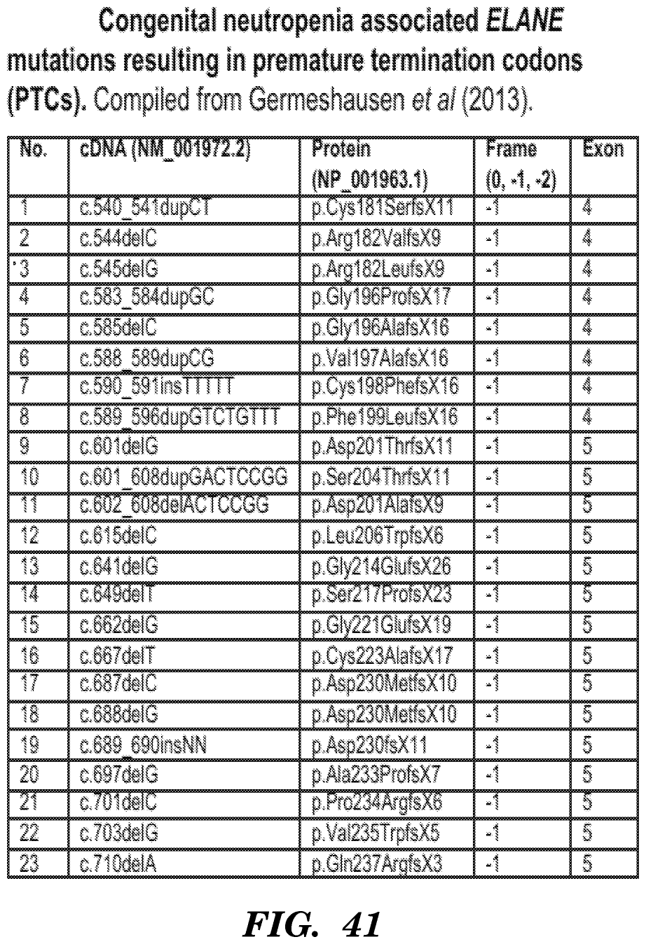

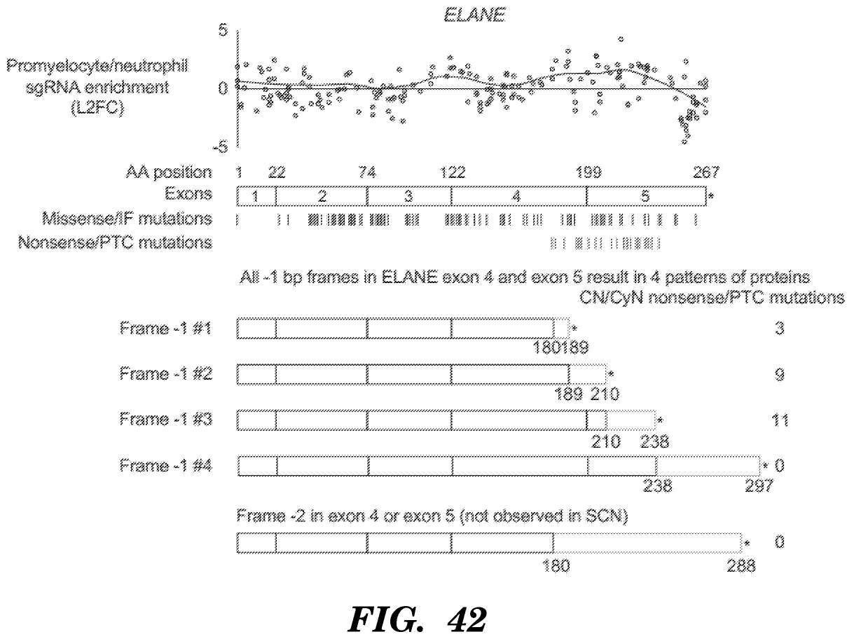

Data presented herein show, for the very first time, a successful ELANE knockout in a mutant ELANE background with the effect of successfully rescuing and overcoming the neutrophil maturation arrest. This is a remarkable result as many of the guide RNA might lead to nonsense-mediated decay of ELANE, however without prospective testing there was no way to know if the approach would work for the gene ELANE and if the residual ELANE protein expressed would be able to overcome the neutrophil maturation arrest. Thus, as described for example in FIG. 10, an unbiased guide RNA pooled screen was needed to identify ELANE-targeting sgRNAs (chimeric single guide RNAs) that are successfully able to target ELANE by nonsense-mediated decay to a residual protein level that allows the ELANE protein to successfully rescue the neutrophil maturation arrest.

### REFERENCES

The contents of all references cited within this document are incorporated herein by reference in their entirety.
1. Prüfer, S. et al. Oxidative burst and neutrophil elastase contribute to clearance of Aspergillus fumigatus pneumonia in mice. Immunobiology 219, 87-96 (2014).
2. Sorensen, O. E. et al. Papillon-Lefevre syndrome patient reveals species-dependent requirements for neutrophil defenses. J. Clin. Invest. 124, 4539-4548 (2014).
3. Tebas, P. et al. Gene Editing of CCR5 in Autologous CD4 T Cells of Persons Infected with HIV. N. Engl. J. Med. 370, 901-910 (2014).
4. Canver, M. C. et al. BCL11A enhancer dissection by Cas9-mediated in situ saturating mutagenesis. Nature 527, 192-197 (2015).
5. Neutrophil Elastase Inhibitors and Chronic Kidney Disease.
Bronze-da-Rocha E, Santos-Silva A.
Int J Biol Sci. 2018 Jul. 27;14(10):1343-1360.
6. Diazaborines as New Inhibitors of Human Neutrophil Elastase.
Antonio J P M, Gonçalves L M, Guedes R C, Moreira R, Gois P M P.
A C S Omega. 2018 Jul. 31;3(7):7418-7423.
7. Synthesis, biological evaluation, and molecular modelling studies of potent human neutrophil elastase (HNE) inhibitors.
Giovannoni M P, Schepetkin I A, Quinn M T, Cantini N, Crocetti L, Guerrini G, Iacovone A, Paoli P,
Rossi P, Bartolucci G, Menicatti M, Vergelli C.
J Enzyme Inhib Med Chem. 2018 December;33(1):1108-1124.
8. Neutrophil elastase inhibitors as potential anti-inflammatory therapies.
Abdel-Magid A F.
ACS Med Chem Lett. 2014 Sep. 8;5(11):1182-3.
9. Targeting COPD: advances on low-molecular-weight inhibitors of human neutrophil elastase.
Lucas S D, Costa E, Guedes R C, Moreira R.
Med Res Rev. 2013 June;33 Suppl 1:E73-101.
10. Genome-scale measurement of off-target activity using Cas9 toxicity in high-throughput screens.
Morgens D W, Wainberg M, Boyle E A, Ursu O, Araya C L, Tsui C K, Haney M S, Hess G T, Han K, Jeng E E, Li A, Snyder M P, Greenleaf W J, Kundaje A, Bassik M C. Nat Commun. 2017 May 5;8:15178. doi: 10.1038/ncomms15178. PMID: 28474669.
11. Germeshausen M, Deerberg S, Peter Y, Reimer C, Kratz C P, Ballmaier M. The spectrum of ELANE mutations and their implications in severe congenital and cyclic neutropenia. Hum Mutat. 2013 June;34(6):905-14.

Table 1 presents sgRNAs tested using methods described herein to target the ELANE gene for knockdown. In Table 1, “ELANE” refers to a sgRNA that is specifically designed to target the ELANE gene. “AAV” refers to a sgRNA that specifically targets the AAV genome. “Safe guides” refer to a sgRNA that is a control, e.g., does not target an essential gene. Safe guides are further described in, e.g., Morgens D W, et al. Nat Commun. 2017 May 5;8:15178. doi: 10.1038/ncomms15178. PMID: 28474669, which is incorporated herein by reference in its entirety.

sgRNA were assigned a functional score that measured its utility in targeting the ELANE gene for degradation. To determine is the sgRNA targeted the ELANE gene, the ratio of promyelocyte to neutrophil enrichment was measured in a cell following contact with a given sgRNA. A negative fold change indicates that the sgRNA is efficient in targeting the ELANE gene for degradation.

In one embodiment, the sgRNA for targeting the ELANE gene has a promyelocyte to neutrophil enrichment fold change of at least −4, −3.5, −3, −2.5, −2, −1.5, −1, −0.5, 0, 0.5 1, 1.5, 2, 2.5, 3, 3.5, 4, 4.5, 5, or more. In a preferred embodiment, the sgRNA for targeting the ELANE gene has a promyelocyte to neutrophil enrichment fold change that is less than 0.

TABLE

| | | | Promyelocyte to |
|---|---|---|---|
| SEQ | | | neutrophil enrichment |
| ID NO: | sgRNA name | Sequence | (log 2 fold change) |
| 1 | ELANE_Exon5_43 | GGGTCCGGGTCCCGGGGGTG | -4.1397 |
| 2 | ELANE_Exon5_47 | CTGGCCGGGTCCGGGTCCCG | -3.6195 |
| 3 | ELANE_Exon3_44 | CCGGGCGGGGCGGGGGGCGC | -3.5373 |
| 4 | ELANE_Exon3_43 | GTGCCGCCGGGCGGGGCGGG | -3.2839 |
| 5 | ELANE_Exon3_39 | CCAGGTGCCGCCGGGCGGGG | -3.1967 |
| 6 | ELANE_Exon5_45 | CCGGGTCCGGGTCCCGGGGG | -3.1546 |
| 7 | ELANE_Exon3_30 | CCGCCCCGCCCGGCGGCACC | -3.0634 |
| 8 | ELANE_Exon3_41 | AGGTGCCGCCGGGCGGGGCG | -2.8821 |
| 9 | ELANE_Exon1_30 | TAAGAGGAGCCGGGCGGGCA | -2.75 |
| 10 | ELANE_Exon5_44 | CGGGTCCGGGTCCCGGGGGT | -2.722 |
| 11 | ELANE_Exon5_42 | CGGGTCCCGGGGGTGGGGAC | -2.6656 |
| 12 | Safe_guide_21 | GAGGAACAGATTCATGAAAG | -2.5845 |
| 13 | ELANE_Exon3_4 | GAGCCCCGCCTCTCCCTCCC | -2.4002 |
| 14 | ELANE_Exon3_24 | GCGGCGGGAGCCCACCCGGC | -2.3865 |
| 15 | ELANE_Exon3_42 | GGTGCCGCCGGGCGGGGCGG | -2.3447 |
| 16 | ELANE_Exon5_41 | GGGTCCCGGGGGTGGGGACA | -2.2908 |
| 17 | ELANE_Exon5_54 | CTGTCCCCACCCCCGGGACC | -2.1574 |
| 18 | ELANE_Exon3_15 | GGGTGGGCTCCCGCCGCGAG | -2.1498 |
| 19 | ELANE_Exon3_40 | CAGGTGCCGCCGGGCGGGGC | -2.1393 |
| 20 | ELANE_Exon5_40 | GGTCCCGGGGGTGGGGACAG | -2.1267 |
| 21 | ELANE_Exon3_38 | TCTCCAGGTGCCGCCGGGCG | -2.103 |
| 22 | ELANE_Exon5_46 | TGGCCGGGTCCGGGTCCCGG | -2.1017 |
| 23 | ELANE_Exon3_32 | GCGCCCCCCGCCCCGCCCGG | -2.0943 |
| 24 | ELANE_Exon5_59 | GGGACCCGGACCCGGCCAGC | -2.0889 |
| 25 | ELANE_Exon3_33 | CCTGCGCCCCCCGCCCCGCC | -2.0457 |
| 26 | Safe_guide_60 | GAATTACATTTCTGCTAAGA | -1.9501 |
| 27 | ELANE_Exon2_16 | TTGTGGGGGGCCGGCGAGCG | -1.8858 |
| 28 | ELANE_Exon5_49 | TGCTGGCCGGGTCCGGGTCC | -1.8732 |
| 29 | ELANE_Exon3_5 | CCAGGACCACCCGCACCGCG | -1.8306 |
| 30 | Safe_guide_73 | GCAATTTATATTTGAGTTCA | -1.8089 |
| 31 | Safe_guide_75 | GTGTGTAGAAATCATATGTG | -1.6479 |
| 32 | ELANE_Exon5_12 | GGGGGACTCCGGCAGCCCCT | -1.6479 |
| 33 | ELANE_Exon5_57 | CCACCCCCGGGACCCGGACC | -1.6479 |
| 34 | Safe_guide_2 | GCACTTCCAGAAAATGCAGC | -1.6446 |
| 35 | ELANE_Exon5_60 | CCCGGGCAGCCCTTCTCAGT | -1.638 |
| 36 | Safe_guide_94 | GAGTCACTAAAAAGAAATAA | -1.6365 |
| 37 | Safe_guide_69 | GTGCCCAGACATTACCTTAA | -1.6017 |
| 38 | ELANE_Exon2_37 | CATGTCGGCCGCGCACTGCG | -1.5988 |
| 39 | Safe_guide_11 | GTAAGAATTTAATGAGCCTC | -1.5914 |
| 40 | Safe_guide_20 | GGATGATTATAATTCTCTAA | -1.5774 |
| 41 | ELANE_Exon5_48 | GCTGGCCGGGTCCGGGTCCC | -1.5499 |
| 42 | ELANE_Exon3_14 | GCTCCCGCCGCGAGAGGTTA | -1.5333 |
| 43 | Safe_guide_83 | GTGAGAGAAGAACTCTCTCT | -1.5167 |
| 44 | ELANE_Exon4_13 | CCCAGGCGGCGTCCCTGAGC | -1.4996 |
| 45 | Safe_guide_40 | GTTGGGGACATAGTTTGATT | -1.4986 |
| 46 | ELANE_Exon3_45 | CGGGCGGGGCGGGGGGCGCA | -1.4954 |
| 47 | ELANE_Exon1_50 | AGGCCGGCAGGACACAGGCG | -1.4736 |
| 48 | ELANE_Exon4_50 | CGAAACAGACGCCGGCCTGC | -1.4561 |
| 49 | Safe_guide_39 | GTGCTATTCGCATATAGGGC | -1.4356 |
| 50 | Safe_guide_84 | GTCCAAGAGACTCATAAAGA | -1.3555 |
| 51 | ELANE_Exon1_15 | TGGAGGGCGCTGGCCGGCCG | -1.3185 |
| 52 | ELANE_Exon2_20 | GGCGAGCGCGGCCCCACGCG | -1.3042 |
| 53 | Safe_guide_82 | GACCTTTCTTTGAAGAAAAC | -1.3006 |
| 54 | ELANE_Exon3_34 | CGTGATTCTCCAGGTGCCGC | -1.2535 |
| 55 | Safe_guide_32 | GAGCGTACTATATTCTTAAA | -1.2386 |
| 56 | ELANE_Exon2_27 | GTGTCCCTGCAGCTGCGCGG | -1.2125 |
| 57 | ELANE_Exon1_27 | ACAGGGCTATAAGAGGAGCC | -1.2091 |
| 58 | ELANE_Exon1_11 | GGAGAGGAAGTGGAGGGCGC | -1.2089 |
| 59 | ELANE_Exon4_26 | GCCCAGAAGGCCCCAGCCCA | -1.1906 |
| 60 | ELANE_Exon1_46 | GACACGCGAGTCGGCGGCCG | -1.1856 |
| 61 | Safe_guide_72 | GATGGTGTTCAAGCTTTCTA | -1.1853 |
| 62 | ELANE_Exon4_55 | GGGGAGCAGAGGGACACCCA | -1.1606 |
| 63 | ELANE_Exon3_7 | GCAGAAACGTCCGCGCGGTG | -1.1588 |
| 64 | ELANE_Exon4_16 | GTGGCCCAGCTGCCGGCTCA | -1.1507 |
| 65 | AAVS1_4 | GTGGGGGTTAGACGAATATC | -1.0685 |
| 66 | Safe_guide_24 | GAAAATGGAGTATAGGGTTT | -1.0313 |
| 67 | ELANE_Exon1_55 | AAACTCACCCCCCAGCAGCA | -0.98211 |
| 68 | ELANE_Exon5_25 | GTCCGGGGAGGCTGCGCCTC | -0.98071 |
| 69 | ELANE_Exon1_29 | GGCTATAAGAGGAGCCGGGC | -0.97734 |
| 70 | ELANE_Exon5_56 | TCTCAGTGGGTCCTGCTGGC | -0.9732 |
| 71 | ELANE_Exon3_36 | ATTCTCCAGGTGCCGCCGGG | -0.97221 |
| 72 | ELANE_Exon1_45 | ACACGCGAGTCGGCGGCCGA | -0.95026 |
| 73 | Safe_guide_37 | GCCCTTCTCCTCATATGGTT | -0.94508 |
| 74 | ELANE_Exon4_19 | GCCGGCTCAGGGACGCCGCC | -0.91736 |
| 75 | ELANE_Exon5_26 | TCCGGGGAGGCTGCGCCTCA | -0.91012 |
| 76 | ELANE_Exon2_24 | GTGGCCTCCGCGCAGCTGCA | -0.88867 |
| 77 | ELANE_Exon2_14 | ACGCGTGGGGCCGCGCTCGC | -0.8885 |
| 78 | ELANE_Exon2_22 | CAGCTGCAGGGACACCATGA | -0.88051 |
| 79 | ELANE_Exon3_37 | TTCTCCAGGTGCCGCCGGGC | -0.85769 |
| 80 | ELANE_Exon1_13 | AGGAAGTGGAGGGCGCTGGC | -0.85552 |
| 81 | ELANE_Exon1_14 | GAGGCCGTTGCATTGCCCCA | -0.84069 |
| 82 | ELANE_Exon1_33 | GGAGCCGGGCGGGCACGGAG | -0.82418 |
| 83 | Safe_guide_64 | GGTAAAGTCAGATAAAAATA | -0.82191 |
| 84 | ELANE_Exon4_15 | GGTGGCCCAGCTGCCGGCTC | -0.80897 |
| 85 | ELANE_Exon5_55 | CTCAGTGGGTCCTGCTGGCC | -0.7864 |
| 86 | ELANE_Exon2_28 | TCAGGGTGGCGCCGCAGAAG | -0.78412 |
| 87 | ELANE_Exon5_52 | GGGTCCTGCTGGCCGGGTCC | -0.76636 |
| 88 | ELANE_Exon1_60 | CTGCCGGCCTTGCTGCTGGG | -0.76428 |
| 89 | ELANE_Exon4_54 | CTCGTGAGGGGCCGGCAGGC | -0.76044 |
| 90 | ELANE_Exon3_1 | CCGCGCGGACGTTTCTGCCG | -0.75887 |
| 91 | Safe_guide_77 | GTTATAAAAGTCTTATGGAT | -0.75701 |
| 92 | ELANE_Exon4_17 | ACTGCACCCCGTTGCCCAGG | -0.75532 |
| 93 | ELANE_Exon4_8 | CAGCTGGGCCACCTGCACGT | -0.72901 |
| 94 | ELANE_Exon5_13 | GGCAGCCCCTTGGTCTGCAA | -0.72076 |
| 95 | ELANE_Exon5_2 | GGGCTGCCGGAGTCCCCCTG | -0.71158 |
| 96 | Safe_guide_41 | GTGACTTTATCCTAAAGGAA | -0.70151 |
| 97 | Safe_guide_43 | GGCACCTTTACATTATGTAA | -0.68646 |
| 98 | Safe_guide_46 | GTTTCCAATAAATAGTCACG | -0.67759 |
| 99 | ELANE_Exon5_1 | CTGCCGGAGTCCCCCTGTGG | -0.66064 |
| 100 | Safe_guide_12 | GGCTTTGGAGCCCAAGTGAG | -0.65001 |
| 101 | Safe_guide_66 | GCCCCAGTTATGTCTTATCT | -0.63107 |
| 102 | ELANE_Exon3_20 | CACGGCGAACACCTGCCGGG | -0.62754 |
| 103 | ELANE_Exon1_32 | AGGAGCCGGGCGGGCACGGA | -0.61736 |
| 104 | Safe_guide_50 | GAATATATTTAGCTACTTTC | -0.59858 |
| 105 | Safe_guide_45 | GACTTGACTTATAAGAGGTG | -0.59621 |
| 106 | ELANE_Exon2_26 | ATGGTGTCCCTGCAGCTGCG | -0.58515 |
| 107 | Safe_guide_85 | GAATTCAGTATTAAACAACA | -0.58487 |
| 108 | ELANE_Exon1_16 | GGAGGGCGCTGGCCGGCCGT | -0.57206 |
| 109 | ELANE_Exon5_63 | CCAGCAGGACCCACTGAGAA | -0.55141 |
| 110 | Safe_guide_36 | GATGCTAAAAATCTAAACCT | -0.54292 |
| 111 | ELANE_Exon2_9 | CGCGCTGGCCTCGGAGATTG | -0.54149 |
| 112 | ELANE_Exon4_24 | GAAGGCCCCAGCCCATGGCC | -0.53335 |
| 113 | Safe_guide_95 | GGCCACAGTGAGATGCAGGA | -0.52361 |
| 114 | ELANE_Exon4_36 | GCAGGACGCTGGCGATCCCA | -0.52047 |
| 115 | ELANE_Exon1_57 | GTCCTGCCGGCCTTGCTGCT | -0.51252 |
| 116 | ELANE_Exon3_13 | CTCCCGCCGCGAGAGGTTAT | -0.51197 |
| 117 | ELANE_Exon3_17 | GAGCCCATAACCTCTCGCGG | -0.49852 |
| 118 | ELANE_Exon2_1 | GACGAAGTTGGGCGCAATCA | -0.49598 |
| 119 | Safe_guide_71 | GAGACATTAGTTAGGAAATA | -0.45528 |
| 120 | ELANE_Exon4_18 | GGCACTGCACCCCGTTGCCC | -0.44827 |
| 121 | ELANE_Exon5_27 | CCACCGGGGCAAAGGCATCG | -0.43383 |
| 122 | ELANE_Exon2_4 | CCCGCACCCGGTGTGTCCCC | -0.43337 |
| 123 | ELANE_Exon5_38 | GTGGGGACAGGGGTTGTCCT | -0.42832 |
| 124 | Safe_guide_47 | GCCTATTCCGTTCATTCAAT | -0.41486 |
| 125 | Safe_guide_53 | GTAAGCTTAAAACATTAGTA | -0.4131 |
| 126 | Safe_guide_33 | GCTCCTCACAGAATTGGCTA | -0.3924 |
| 127 | Safe_guide_29 | GCAGCCAATTATTGCCAAAT | -0.37432 |
| 128 | Safe_guide_65 | GGAAACGGAGGAAGTCCACT | -0.37213 |
| 129 | ELANE_Exon3_9 | AAACGTCCGCGCGGTGCGGG | -0.37057 |
| 130 | ELANE_Exon1_52 | CCCCCAGCAGCAAGGCCGGC | -0.36635 |
| 131 | ELANE_Exon2_8 | CCCAGGCACCGCGCTGGCCT | -0.36483 |
| 132 | Safe_guide_58 | GAATCCACACATAAAAGCAC | -0.35799 |
| 133 | Safe_guide_26 | GGTGGTTAGCCCAGTTCATT | -0.35673 |
| 134 | ELANE_Exon1_19 | TCTTATAGCCCTGTGCTGGG | -0.35336 |
| 135 | ELANE_Exon4_37 | GGCCTTCTGGGCAGGAACCG | -0.35334 |
| 136 | Safe_guide_56 | GCTCATTCGAACTTCTTCCA | -0.3528 |
| 137 | ELANE_Exon3_18 | AGCCCATAACCTCTCGCGGC | -0.34796 |
| 138 | ELANE_Exon4_33 | GGCCATGGGCTGGGGCCTTC | -0.3477 |
| 139 | ELANE_Exon5_65 | TGTGGGCAGCTGAGGTGACC | -0.32691 |
| 140 | Safe_guide_30 | GTTAGAATCCATAACTTCTC | -0.32313 |
| 141 | ELANE_Exon2_15 | CCTCGGAGATTGTGGGGGGC | -0.31477 |
| 142 | ELANE_Exon3_19 | ACGGCGAACACCTGCCGGGT | -0.30328 |
| 143 | ELANE_Exon5_53 | TGGGTCCTGCTGGCCGGGTC | -0.29721 |
| 144 | Safe_guide_80 | GTCTCTAGATAGTGTAAATG | -0.28142 |
| 145 | Safe_guide_14 | GTTTGTAACTTATGGTACTG | -0.25275 |
| 146 | ELANE_Exon3_35 | GTGATTCTCCAGGTGCCGCC | -0.24275 |
| 147 | Safe_guide_54 | GTAGACACAAACTTGTGTAA | -0.23967 |
| 148 | ELANE_Exon1_59 | CCTGCCGGCCTTGCTGCTGG | -0.21611 |
| 149 | ELANE_Exon2_7 | CCGGCCCCCCACAATCTCCG | -0.20574 |
| 150 | Safe_guide_92 | GACAACCTCACAGAAAATAA | -0.19698 |
| 151 | ELANE_Exon1_36 | TCATGGTGGGGCTGGGGCTC | -0.19373 |
| 152 | Safe_guide_19 | GAGCACTTAATATGTGCAAA | -0.19098 |
| 153 | ELANE_Exon3_21 | CTGCACGGCGAACACCTGCC | -0.1763 |
| 154 | ELANE_Exon2_5 | CACAATCTCCGAGGCCAGCG | -0.16581 |
| 155 | Safe_guide_49 | GATCAATACTTCACAGTCCT | -0.15919 |
| 156 | Safe_guide_35 | GCACTAACATTCATAAATAT | -0.14909 |
| 157 | Safe_guide_31 | GGTTGTGAATACATAGTAAT | -0.13713 |
| 158 | Safe_guide_34 | GAAAGTTAATATCTTTAGCT | -0.13364 |
| 159 | ELANE_Exon2_25 | AGTGGCCTCCGCGCAGCTGC | -0.13113 |
| 160 | ELANE_Exon4_27 | CGGGGTGCAGTGCCTGGCCA | -0.12099 |
| 161 | ELANE_Exon4_11 | GCGTCCCTGAGCCGGCAGCT | -0.11482 |
| 162 | ELANE_Exon4_35 | TGGGCTGGGGCCTTCTGGGC | -0.10731 |
| 163 | ELANE_Exon2_29 | CTGCGCGGAGGCCACTTCTG | -0.1011 |
| 164 | ELANE_Exon5_14 | GCAGCCCCTTGGTCTGCAAC | -0.068585 |
| 165 | ELANE_Exon4_25 | GGGCAACGGGGTGCAGTGCC | -0.043547 |
| 166 | Safe_guide_67 | GACATTTGACACTGAAGCTA | -0.042971 |
| 167 | ELANE_Exon3_11 | CCGCGCGGTGCGGGTGGTCC | -0.024879 |
| 168 | Safe_guide_13 | GAATTAAATTCACTTTGAAC | -0.019057 |
| 169 | Safe_guide_7 | GTACTTCCACTGTCACTTTG | 0.00098329 |
| 170 | ELANE_Exon2_10 | GCGCTGGCCTCGGAGATTGT | 0.010328 |
| 171 | Safe_guide_22 | GTGTAAAGCTTCCACTTCAC | 0.018683 |
| 172 | Safe_guide_81 | GTTTATTTGCAAGGAATACA | 0.019185 |
| 173 | ELANE_Exon2_6 | TGTGTCCCCAGGCACCGCGC | 0.024132 |
| 174 | ELANE_Exon4_23 | GGGACGCCGCCTGGGCAACG | 0.026274 |
| 175 | Safe_guide_62 | GCTCTACATAGGTAACAGGA | 0.04869 |
| 176 | ELANE_Exon4_12 | GGCGTCCCTGAGCCGGCAGC | 0.05781 |
| 177 | Safe_guide_51 | GTCATCAGAAGAAACCTCTT | 0.069938 |
| 178 | ELANE_Exon1_28 | GGGCTATAAGAGGAGCCGGG | 0.070273 |
| 179 | ELANE_Exon1_35 | CATGGTGGGGCTGGGGCTCC | 0.072785 |
| 180 | ELANE_Exon3_8 | CAGAAACGTCCGCGCGGTGC | 0.073342 |
| 181 | Safe_guide_28 | GTTCAAACAACATACTGAAA | 0.076031 |
| 182 | Safe_guide_52 | GAATTGAGGCAGTAGCCTCT | 0.081844 |
| 183 | Safe_guide_42 | GAGGGGACCCTGGTTTGAGG | 0.092683 |
| 184 | Safe_guide_5 | GTGAGTTTCTTTCTGCCAGG | 0.094543 |
| 185 | ELANE_Exon1_26 | CACAGGGCTATAAGAGGAGC | 0.10338 |
| 186 | ELANE_Exon4_41 | TGGGATCGCCAGCGTCCTGC | 0.10701 |
| 187 | Safe_guide_27 | GTGAGCTCCTCATTCATTCC | 0.10823 |
| 188 | ELANE_Exon5_5 | CACCTTGTCTGCCTCCACAG | 0.15759 |
| 189 | ELANE_Exon3_12 | CGCGCGGTGCGGGTGGTCCT | 0.17026 |
| 190 | ELANE_Exon3_26 | GTGCAGCGCATCTTCGAAAA | 0.18084 |
| 191 | Safe_guide_6 | GTGTTCTCAGTTATATGCAA | 0.18267 |
| 192 | ELANE_Exon4_52 | CACTCTCGTGAGGGGCCGGC | 0.18579 |
| 193 | Safe_guide_63 | GGCCACATTAATAACCTTAC | 0.19712 |
| 194 | Safe_guide_74 | GTAAAAATGTCCTTGTCACC | 0.209 |
| 195 | Safe_guide_86 | GTTACTGAAGTAACTTGCCT | 0.21228 |
| 196 | Safe_guide_79 | GGTGTGACAAGGAGAACAAA | 0.2272 |
| 197 | ELANE_Exon3_22 | GCTGCACGGCGAACACCTGC | 0.24061 |
| 198 | ELANE_Exon2_30 | GAAGTTGGGCGCAATCAGGG | 0.2607 |
| 199 | ELANE_Exon1_25 | TCTGCCCCTCCGTGCCCGCC | 0.28002 |
| 200 | ELANE_Exon5_70 | GATGCTGGAGAGTGTGGGTG | 0.29448 |
| 201 | Safe_guide_89 | GTGCACTACAAGTAATACTA | 0.30497 |
| 202 | ELANE_Exon1_17 | GAGGGCGCTGGCCGGCCGTG | 0.31435 |
| 203 | ELANE_Exon1_39 | CCGAGGGTCATGGTGGGGCT | 0.33294 |
| 204 | ELANE_Exon4_56 | CGTCTGTTTCGTACGTGCCC | 0.33426 |
| 205 | ELANE_Exon2_32 | TGACGAAGTTGGGCGCAATC | 0.3397 |
| 206 | ELANE_Exon1_53 | TCACCCCCCAGCAGCAAGGC | 0.34037 |
| 207 | ELANE_Exon4_20 | CCGGCTCAGGGACGCCGCCT | 0.34229 |
| 208 | Safe_guide_15 | GTGGAATCATTACTCCAAAA | 0.35834 |
| 209 | Safe_guide_1 | GAAACAAGCTAATATTATCA | 0.37072 |
| 210 | ELANE_Exon4_21 | CAGGGACGCCGCCTGGGCAA | 0.37667 |
| 211 | ELANE_Exon5_74 | CACCCACACTCTCCAGCATC | 0.38459 |
| 212 | ELANE_Exon2_13 | CTGGCCTCGGAGATTGTGGG | 0.39043 |
| 213 | ELANE_Exon2_33 | GCGGCCGACATGACGAAGTT | 0.39332 |
| 214 | ELANE_Exon1_4 | TTTCATCAACGCCCTGTGCC | 0.40123 |
| 215 | Safe_guide_16 | GTATCGTAAATGGATGTAGA | 0.45936 |
| 216 | ELANE_Exon1_31 | GAGGAGCCGGGCGGGCACGG | 0.46559 |
| 217 | ELANE_Exon1_48 | GGAAAAGACACGCGAGTCGG | 0.4736 |
| 218 | AAVS1_2 | CCGAATTGGAGCGCTTCAAC | 0.48111 |
| 219 | Safe_guide_61 | GAAGCAGGAGGTGTACCAGT | 0.48454 |
| 220 | ELANE_Exon2_36 | CACATTCGCCACGCAGTGCG | 0.48657 |
| 221 | ELANE_Exon1_1 | ACTTCCTCTCCCCTGGCACA | 0.49873 |
| 222 | ELANE_Exon5_69 | ATGCTGGAGAGTGTGGGTGT | 0.5049 |
| 223 | ELANE_Exon2_12 | GCTGGCCTCGGAGATTGTGG | 0.50778 |
| 224 | Safe_guide_76 | GCTGCATGCTGGCCAGCAGC | 0.52747 |
| 225 | ELANE_Exon1_7 | AACGCCCTGTGCCAGGGGAG | 0.55476 |
| 226 | ELANE_Exon1_21 | CTCCTCTTATAGCCCTGTGC | 0.56812 |
| 227 | ELANE_Exon4_38 | GCCTTCTGGGCAGGAACCGT | 0.57476 |
| 228 | ELANE_Exon5_64 | GTGGGCAGCTGAGGTGACCC | 0.58185 |
| 229 | ELANE_Exon4_29 | TGCAGTGCCTGGCCATGGGC | 0.58281 |
| 230 | ELANE_Exon2_21 | AGCTGCAGGGACACCATGAA | 0.58364 |
| 231 | Safe_guide_68 | GACGTGAGCTACAATGGGAA | 0.60452 |
| 232 | ELANE_Exon5_19 | GCCCTGAGGCGCAGCCTCCC | 0.6067 |
| 233 | ELANE_Exon1_44 | GAGTCGGCGGCCGAGGGTCA | 0.60765 |
| 234 | ELANE_Exon3_16 | TGGGAGCCCATAACCTCTCG | 0.61325 |
| 235 | ELANE_Exon1_6 | TCATCAACGCCCTGTGCCAG | 0.6233 |
| 236 | ELANE_Exon3_10 | GCGAGAGGTTATGGGCTCCC | 0.62594 |
| 237 | ELANE_Exon5_72 | GTGCCAGATGCTGGAGAGTG | 0.63204 |
| 238 | ELANE_Exon1_38 | CGAGGGTCATGGTGGGGCTG | 0.64114 |
| 239 | Safe_guide_91 | GATCACAAACTCATCTTTCA | 0.6464 |
| 240 | ELANE_Exon4_34 | GCCATGGGCTGGGGCCTTCT | 0.65167 |
| 241 | Safe_guide_17 | GGAAAACACTGTTCAACTTA | 0.6546 |
| 242 | ELANE_Exon5_61 | ACCCGGGCAGCCCTTCTCAG | 0.672 |
| 243 | Safe_guide_88 | GCAGGGAATGGAAATGAATA | 0.67566 |
| 244 | ELANE_Exon4_28 | GGGGTGCAGTGCCTGGCCAT | 0.69518 |
| 245 | Safe_guide25 | GCATAGTTAGTATGGGACTT | 0.6988 |
| 246 | ELANE_Exon3_25 | GTTTTCGAAGATGCGCTGCA | 0.71607 |
| 247 | Safe_guide_3 | GAAAATCTTACACATGGATA | 0.71771 |
| 248 | Safe_guide_57 | GGACATAAGAAGTTGATAGT | 0.72367 |
| 249 | ELANE_Exon4_43 | GCAGGAGCTCAACGTGACGG | 0.73518 |
| 250 | ELANE_Exon3_2 | ACCGCGCGGACGTTTCTGCC | 0.73976 |
| 251 | Safe_guide_8 | GTTAAATTATGGTGATTCTA | 0.75107 |
| 252 | ELANE_Exon4_57 | GTCTGTTTCGTACGTGCCCT | 0.75373 |
| 253 | Safe_guide_18 | GTCTTTCACGATGTACCCTA | 0.76536 |
| 254 | Safe_guide_59 | GAAATAACATACGACTATAC | 0.76711 |
| 255 | ELANE_Exon4_40 | CCGTCACGTTGAGCTCCTGC | 0.81387 |
| 256 | Safe_guide_55 | GGAGGATCACAGCAAGCTTC | 0.81903 |
| 257 | ELANE_Exon3_27 | TGTCGTTGAGCAAGTTTACG | 0.83785 |
| 258 | ELANE_Exon5_4 | CCACCTTGTCTGCCTCCACA | 0.84191 |
| 259 | Safe_guide_93 | GCATCATAGCAAGAAGAGTT | 0.85684 |
| 260 | ELANE_Exon5_58 | CCCTTCTCAGTGGGTCCTGC | 0.86918 |
| 261 | ELANE_Exon5_39 | CTCTATCATCCAACGCTCCG | 0.88564 |
| 262 | ELANE_Exon2_17 | ACCATGAAGGGCCACGCGTG | 0.89484 |
| 263 | ELANE_Exon1_5 | TTCATCAACGCCCTGTGCCA | 0.89547 |
| 264 | ELANE_Exon3_3 | CACCGCGCGGACGTTTCTGC | 0.91893 |
| 265 | ELANE_Exon5_30 | AAACTGTGCCACCGGGGCAA | 0.9259 |
| 266 | ELANE_Exon4_42 | CCTGCAGGAGCTCAACGTGA | 0.94095 |
| 267 | ELANE_Exon5_7 | GTTGCAGACCAAGGGGCTGC | 0.97021 |
| 268 | Safe_guide_87 | GAAGATAATGTCTGAGTGAT | 0.97837 |
| 269 | ELANE_Exon1_37 | GCACGGAGGGGCAGAGACCC | 0.98293 |
| 270 | ELANE_Exon5_66 | ACCCACTGAGAAGGGCTGCC | 0.98323 |
| 271 | ELANE_Exon2_1 | CGAGGCCAGCGCGGTGCCTG | 0.99801 |
| 272 | ELANE_Exon1_41 | GGCGGCCGAGGGTCATGGTG | 0.99994 |
| 273 | ELANE_Exon3_6 | CCCCGGCAGAAACGTCCGCG | 1.0787 |
| 274 | ELANE_Exon1_43 | TCGGCGGCCGAGGGTCATGG | 1.0878 |
| 275 | ELANE_Exon5_62 | GCCAGCAGGACCCACTGAGA | 1.1012 |
| 276 | AAVS1_5 | AGTTCTTAGGTACCCCACGT | 1.1364 |
| 277 | ELANE_Exon1_18 | CCGGCCGTGGGGCAATGCAA | 1.1442 |
| 278 | ELANE_Exon1_58 | TCCTGCCGGCCTTGCTGCTG | 1.1618 |
| 279 | ELANE_Exon5_73 | ATGTTTATTGTGCCAGATGC | 1.1723 |
| 280 | ELANE_Exon1_22 | ATGCAACGGCCTCCCAGCAC | 1.1741 |
| 281 | ELANE_Exon4_51 | TCTGCACTCTCGTGAGGGGC | 1.197 |
| 282 | ELANE_Exon1_20 | TCCTCTTATAGCCCTGTGCT | 1.2274 |
| 283 | ELANE_Exon4_44 | GACGTTGCTGCGACGGCAGA | 1.2274 |
| 284 | ELANE_Exon4_53 | GGCACGTACGAAACAGACGC | 1.2309 |
| 285 | Safe_guide_9 | GATCAACTCTCATCATATAC | 1.241 |
| 286 | ELANE_Exon2_2 | CCGAGGCCAGCGCGGTGCCT | 1.2533 |
| 287 | ELANE_Exon1_2 | CACTTCCTCTCCCCTGGCAC | 1.2579 |
| 288 | ELANE_Exon4_22 | AGGGACGCCGCCTGGGCAAC | 1.2787 |
| 289 | ELANE_Exon4_2 | GTGGCCGACCCGTTGAGCTG | 1.296 |
| 290 | ELANE_Exon1_42 | CGGCGGCCGAGGGTCATGGT | 1.2995 |
| 291 | ELANE_Exon5_17 | GCAGCCTCCCCGGACGAAGG | 1.3197 |
| 292 | ELANE_Exon5_37 | AGGGGTTGTCCTCGGAGCGT | 1.3202 |
| 293 | ELANE_Exon4_39 | GTTGAGCTCCTGCAGGACGC | 1.3398 |
| 294 | ELANE_Exon2_23 | GCCCCACGCGTGGCCCTTCA | 1.3768 |
| 295 | ELANE_Exon5_35 | CCCCGATGCCTTTGCCCCGG | 1.4046 |
| 296 | ELANE_Exon1_40 | GCCGAGGGTCATGGTGGGGC | 1.4065 |
| 297 | ELANE_Exon5_50 | ACAACCCCTGTCCCCACCCC | 1.4092 |
| 298 | ELANE_Exon5_22 | GGAATTGCCTCCTTCGTCCG | 1.4181 |
| 299 | ELANE_Exon4_14 | CGTGCAGGTGGCCCAGCTGC | 1.4379 |
| 300 | ELANE_Exon3_31 | CAACGACATCGTGATTCTCC | 1.4764 |
| 301 | Safe_guide_4 | GGAAGTGAGTTAAAAGAAGT | 1.4943 |
| 302 | ELANE_Exon4_32 | TCCCACGGTTCCTGCCCAGA | 1.4977 |
| 303 | ELANE_Exon4_1 | GCCGACCCGTTGAGCTGTGG | 1.5721 |
| 304 | ELANE_Exon4_30 | GCAGTGCCTGGCCATGGGCT | 1.5741 |
| 305 | ELANE_Exon2_34 | CGCGGCCGACATGACGAAGT | 1.602 |
| 306 | Safe_guide_48 | GATCAGCCCATCAAATGAGG | 1.6219 |
| 307 | AAVS1_3 | CCCAGCGAGTGAACACGGCA | 1.6458 |
| 308 | ELANE_Exon5_6 | ACCTTGTCTGCCTCCACAGG | 1.6552 |
| 309 | ELANE_Exon4_10 | CATCAACGCCAACGTGCAGG | 1.6607 |
| 310 | Safe_guide_10 | GTAAAAGATGTGCATTTGAG | 1.6634 |
| 311 | ELANE_Exon3_28 | ATGTCGTTGAGCAAGTTTAC | 1.6663 |
| 312 | ELANE_Exon2_18 | CACCATGAAGGGCCACGCGT | 1.6857 |
| 313 | ELANE_Exon4_31 | CAGTGCCTGGCCATGGGCTG | 1.7028 |
| 314 | Safe_guide_23 | GACACTACATTACAAACAAC | 1.7053 |
| 315 | Safe_guide_78 | GCCTGCTTCCATGCCTGGTT | 1.7127 |
| 316 | ELANE_Exon5_15 | GGACGAAGGAGGCAATTCCG | 1.7605 |
| 317 | ELANE_Exon5_8 | CTGCCTCCACAGGGGGACTC | 1.7668 |
| 318 | ELANE_Exon2_35 | TGCGCCCAACTTCGTCATGT | 1.7727 |
| 319 | ELANE_Exon5_71 | TGCCAGATGCTGGAGAGTGT | 1.7862 |
| 320 | ELANE_Exon4_7 | CACCTGCACGTTGGCGTTGA | 1.8082 |
| 321 | ELANE_Exon5_21 | CGGAATTGCCTCCTTCGTCC | 1.8146 |
| 322 | ELANE_Exon5_67 | CCCACTGAGAAGGGCTGCCC | 1.8189 |
| 323 | ELANE_Exon1_54 | TTTCCTCGCCTGTGTCCTGC | 1.8341 |
| 324 | ELANE_Exon4_6 | CTGCACGTTGGCGTTGATGG | 1.8585 |
| 325 | ELANE_Exon5_18 | GGCGCAGCCTCCCCGGACGA | 1.8798 |
| 326 | AAVS1_1 | GTGTGTGTAGCACCGCGTAA | 1.881 |
| 327 | ELANE_Exon1_24 | TCCCAGCACAGGGCTATAAG | 1.9017 |
| 328 | ELANE_Exon5_3 | CCCACCTTGTCTGCCTCCAC | 1.9356 |
| 329 | Safe_guide_90 | GTCTTGACTGCTAGAATGTA | 1.9478 |
| 330 | ELANE_Exon5_31 | GTTTACAAACTGTGCCACCG | 1.981 |
| 331 | ELANE_Exon5_36 | CGGTGGCACAGTTTGTAAAC | 2.0084 |
| 332 | ELANE_Exon3_23 | TCTCGCGGCGGGAGCCCACC | 2.0304 |
| 333 | ELANE_Exon4_45 | AGACGTTGCTGCGACGGCAG | 2.0548 |
| 334 | Safe_guide_70 | GTACTATGTTAACGTATTTC | 2.0716 |
| 335 | ELANE_Exon1_56 | TGTCCTGCCGGCCTTGCTGC | 2.0722 |
| 336 | ELANE_Exon2_11 | CGCTGGCCTCGGAGATTGTG | 2.0794 |
| 337 | ELANE_Exon2_19 | ACACCATGAAGGGCCACGCG | 2.1282 |
| 338 | ELANE_Exon4_3 | TCCCCACCGCCACAGCTCAA | 2.1595 |
| 339 | Safe_guide_44 | GTCCAAGAACAAAGCAAAGA | 2.1737 |
| 340 | ELANE_Exon1_51 | CAGCAAGGCCGGCAGGACAC | 2.1808 |
| 341 | ELANE_Exon5_32 | AGTTTACAAACTGTGCCACC | 2.1834 |
| 342 | ELANE_Exon4_5 | ACCGCCACAGCTCAACGGGT | 2.2085 |
| 343 | ELANE_Exon5_16 | GTCTGCAACGGGCTAATCCA | 2.2128 |
| 344 | ELANE_Exon5_34 | CTACCCCGATGCCTTTGCCC | 2.2323 |
| 345 | ELANE_Exon3_29 | GATGTCGTTGAGCAAGTTTA | 2.249 |
| 346 | ELANE_Exon1_47 | CCCAGCCCCACCATGACCCT | 2.2864 |
| 347 | ELANE_Exon4_47 | GCAACGTCTGCACTCTCGTG | 2.2918 |
| 348 | ELANE_Exon5_29 | TGCCACCGGGGCAAAGGCAT | 2.2934 |
| 349 | ELANE_Exon4_9 | CACCATCAACGCCAACGTGC | 2.3097 |
| 350 | ELANE_Exon5_24 | GGCATCGGGGTAGAGCCCTG | 2.3339 |
| 351 | ELANE_Exon1_12 | CCGTTGCATTGCCCCACGGC | 2.3391 |
| 352 | ELANE_Exon5_33 | CAGTTTACAAACTGTGCCAC | 2.3613 |
| 353 | ELANE_Exon5_11 | GATTAGCCCGTTGCAGACCA | 2.3817 |
| 354 | ELANE_Exon1_49 | CGAGGAAAAGACACGCGAGT | 2.4404 |
| 355 | ELANE_Exon5_20 | ACGGAATTGCCTCCTTCGTC | 2.4657 |
| 356 | ELANE_Exon5_28 | GCCACCGGGGCAAAGGCATC | 2.4811 |
| 357 | ELANE_Exon1_23 | TGCAACGGCCTCCCAGCACA | 2.703 |
| 358 | ELANE_Exon4_49 | AACGTCTGCACTCTCGTGAG | 2.7041 |
| 359 | ELANE_Exon5_51 | CAACCCCTGTCCCCACCCCC | 2.7276 |
| 360 | ELANE_Exon4_46 | GAGTGCAGACGTTGCTGCGA | 2.7894 |
| 361 | ELANE_Exon2_3 | TCCGAGGCCAGCGCGGTGCC | 2.838 |
| 362 | ELANE_Exon1_10 | GCCAGGGGAGAGGAAGTGGA | 3.0216 |
| 363 | ELANE_Exon5_68 | AGTGTGGGTGTGGGCAGCTG | 3.0415 |
| 364 | Safe_guide_38 | GGGAGCCCTGGGCAGGTTCA | 3.0499 |
| 365 | ELANE_Exon4_4 | CCCCACCGCCACAGCTCAAC | 3.0592 |
| 366 | ELANE_Exon1_9 | TGCCAGGGGAGAGGAAGTGG | 3.103 |
| 367 | ELANE_Exon5_10 | ATTAGCCCGTTGCAGACCAA | 3.1634 |
| 368 | ELANE_Exon5_9 | TTAGCCCGTTGCAGACCAAG | 3.1905 |
| 369 | ELANE_Exon1_8 | CTGTGCCAGGGGAGAGGAAG | 3.1976 |
| 370 | ELANE_Exon1_3 | GCCCTCCACTTCCTCTCCCC | 3.5793 |
| 371 | ELANE_Exon4_48 | CAACGTCTGCACTCTCGTGA | 3.6005 |
| 372 | ELANE_Exon5_23 | ATTGCCTCCTTCGTCCGGGG | 4.6167 |
| 373 | ELANE_Exon1_34 | ATGGTGGGGCTGGGGCTCCG | 5.2766 |

what is claimed is:

1. A modified synthetic nucleic acid comprising a nucleic acid sequence shown in Table 1, SEQ ID NOS: 1-374, wherein there is at least one chemical modification to a nucleotide in the nucleic acid molecule.

2. The modified synthetic nucleic acid of claim 1, wherein the at least one chemical modifications is located at one or more terminal nucleotides in nucleic acid molecule.

3. The modified synthetic nucleic acid of claim 2, wherein the at least one chemical modification is selected from the group consisting of 2′-O-methyl 3′phosphorothioate (MS), 2′-O-methyl-3′-phosphonoacetate (MP), 2′-0-Ci-4alkyl, 2′-H, 2′-0-Ci.3alkyl-0-Ci.3alkyl, 2′-F, 2′-NH2, 2′-arabino, 2′- F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphonate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “xDNA” base), an yRNA base (analogous to “yDNA” base), a PEG substituent, and a conjugated linker to a dye or non-fluorescent label (or tag).

4. The modified synthetic nucleic acid of claim 1, wherein the at least one chemical modification is located only at the 3′ end, or added only at the 5′ end, or added at both the 5′ and 3′ ends of the synthetic nucleic acid molecule; or
wherein the at least one chemical modification is located to first three nucleotides and to the last three nucleotides of the synthetic nucleic acid molecule.

5. (canceled)

6. The modified synthetic nucleic acid of claim 1, wherein the modified synthetic nucleic acid sequence further comprising a crRNA/tracrRNA sequence.

7. The modified synthetic nucleic acid of claim 1, wherein the modified synthetic nucleic acid is a single guide RNA (sgRNA).

8.-10. (canceled)

11. The modified synthetic nucleic acid of claim 1, wherein the modified synthetic nucleic acid is used in combination with a DNA-targeting endonuclease Cas (CRISPR-associated) protein in a ribonucleoprotein (RNP) complex.

12. The modified synthetic nucleic acid of claim 11, wherein the Cas protein is Cas 9.

13.-19. (canceled)

20. A composition comprising a modified synthetic nucleic acid molecule of any claim 1.

21.-26. (canceled)

27. A ribonucleoprotein (RNP) complex comprising a DNA-targeting endonuclease Cas (CRISPR-associated) protein and a modified synthetic nucleic acid molecule of claim 1.

28.-31. (canceled)

32. A method for producing a progenitor cell or a population of progenitor cells having decreased ELANE mRNA or protein expression, the method comprising contacting an isolated progenitor cell with an effective amount of a modified synthetic nucleic acid of claim 1, whereby the contacted cells or the differentiated progeny cells therefrom acquires at least one genetic modification resulting in decreased ELANE mRNA or protein expression.

33. The method of claim 32, wherein the at least one genetic modification is a deletion, insertion or substitution of the genetic sequence of the cell.

34. The method of any one of claims 32, wherein the isolated progenitor cell is a hematopoietic progenitor cell, a hematopoietic stem cell, or an induced pluripotent stem cell.

35. The method of claim 34, wherein the hematopoietic progenitor is a cell of the erythroid lineage.

36. (canceled)

37. The method of any claim 32, wherein the isolated progenitor cell is contacted ex vivo or in vitro.

38. (canceled)

39. The method of claim 37, wherein the contacted progenitor cell or contacted cell are further electroporated.

40. The method of claim 39, wherein the step of electroporation is performed in a solution comprising glycerol.

41.-44. (canceled)

45. A composition comprising genetically edited modified progenitor cells of claim 32.

46. A method of treating a disease associated with abnormal ELANE gene expression, the method comprising, administering to a subject in need thereof a gene editing agent that targets the ELANE gene, wherein the edited ELANE gene comprises at least one mutation selected from the group consisting of a loss-of-function mutation, a through stop-gain mutation, a frameshift mutation, a premature termination codon, and a splicing mutation that encodes a premature termination codon,
wherein the gene editing agent is a modified synthetic nucleic acid of claim 1.

47. The method of claim 47, wherein the disease is selected from the group consisting of chronic obstructive pulmonary disease (COPD) (including acquired disease or genetic alpha-1 antitrypsin deficiency), cystic fibrosis, acute respiratory distress syndrome (ARDS), acute lung injury (ALI), asthmatic conditions, rheumatoid arthritis, chronic kidney disease, cyclic neutropenia, and severe congential neutropenia.

48.-51. (canceled)


SEQUENCE LISTING (412 sequences)

SEQ ID NO: 1 - DNA, 20 bp, Artificial Sequence
  Description of Artificial Sequence: Synthetic oligonucleotide
gggtccgggtcccgggggtg

SEQ ID NO: 2 - DNA, 20 bp, Artificial Sequence
  Description of Artificial Sequence: Synthetic oligonucleotide
ctggccgggtccgggtcccg

SEQ ID NO: 3 - DNA, 20 bp, Artificial Sequence
  Description of Artificial Sequence: Synthetic oligonucleotide
ccgggcggggcggggggcgc

SEQ ID NO: 4 - DNA, 20 bp, Artificial Sequence
  Description of Artificial Sequence: Synthetic oligonucleotide
gtgccgccgggcggggcggg

SEQ ID NO: 5 - DNA, 20 bp, Artificial Sequence
  Description of Artificial Sequence: Synthetic oligonucleotide
ccaggtgccgccgggcgggg

SEQ ID NO: 6 - DNA, 20 bp, Artificial Sequence
  Description of Artificial Sequence: Synthetic oligonucleotide
ccgggtccgggtcccggggg

SEQ ID NO: 7 - DNA, 20 bp, Artificial Sequence
  Description of Artificial Sequence: Synthetic oligonucleotide
ccgccccgcccggcggcacc

SEQ ID NO: 8 - DNA, 20 bp, Artificial Sequence
  Description of Artificial Sequence: Synthetic oligonucleotide
aggtgccgccgggcggggcg

SEQ ID NO: 9 - DNA, 20 bp, Artificial Sequence
  Description of Artificial Sequence: Synthetic oligonucleotide
taagaggagccgggcgggca

SEQ ID NO: 10 - DNA, 20 bp, Artificial Sequence
  Description of Artificial Sequence: Synthetic oligonucleotide
cgggtccgggtcccgggggt

SEQ ID NO: 11 - DNA, 20 bp, Artificial Sequence
  Description of Artificial Sequence: Synthetic oligonucleotide
cgggtcccgggggtggggac

SEQ ID NO: 12 - DNA, 20 bp, Artificial Sequence
  Description of Artificial Sequence: Synthetic oligonucleotide
gaggaacagattcatgaaag

SEQ ID NO: 13 - DNA, 20 bp, Artificial Sequence
  Description of Artificial Sequence: Synthetic oligonucleotide
gagccccgcctctccctccc

SEQ ID NO: 14 - DNA, 20 bp, Artificial Sequence
  Description of Artificial Sequence: Synthetic oligonucleotide
gcggcgggagcccacccggc

SEQ ID NO: 15 - DNA, 20 bp, Artificial Sequence
  Description of Artificial Sequence: Synthetic oligonucleotide
ggtgccgccgggcggggcgg

SEQ ID NO: 16 - DNA, 20 bp, Artificial Sequence
  Description of Artificial Sequence: Synthetic oligonucleotide
gggtcccgggggtggggaca

SEQ ID NO: 17 - DNA, 20 bp, Artificial Sequence
  Description of Artificial Sequence: Synthetic oligonucleotide
ctgtccccacccccgggacc

SEQ ID NO: 18 - DNA, 20 bp, Artificial Sequence
  Description of Artificial Sequence: Synthetic oligonucleotide
gggtgggctcccgccgcgag

SEQ ID NO: 19 - DNA, 20 bp, Artificial Sequence
  Description of Artificial Sequence: Synthetic oligonucleotide
caggtgccgccgggcggggc

SEQ ID NO: 20 - DNA, 20 bp, Artificial Sequence
  Description of Artificial Sequence: Synthetic oligonucleotide
ggtcccgggggtggggacag

SEQ ID NO: 21 - DNA, 20 bp, Artificial Sequence
  Description of Artificial Sequence: Synthetic oligonucleotide
tctccaggtgccgccgggcg

SEQ ID NO: 22 - DNA, 20 bp, Artificial Sequence
  Description of Artificial Sequence: Synthetic oligonucleotide
tggccgggtccgggtcccgg

SEQ ID NO: 23 - DNA, 20 bp, Artificial Sequence
  Description of Artificial Sequence: Synthetic oligonucleotide
gcgccccccgccccgcccgg

SEQ ID NO: 24 - DNA, 20 bp, Artificial Sequence
  Description of Artificial Sequence: Synthetic oligonucleotide
gggacccggacccggccagc

SEQ ID NO: 25 - DNA, 20 bp, Artificial Sequence
  Description of Artificial Sequence: Synthetic oligonucleotide
cctgcgccccccgccccgcc

SEQ ID NO: 26 - DNA, 20 bp, Artificial Sequence
  Description of Artificial Sequence: Synthetic oligonucleotide
gaattacatttctgctaaga

SEQ ID NO: 27 - DNA, 20 bp, Artificial Sequence
  Description of Artificial Sequence: Synthetic oligonucleotide
ttgtggggggccggcgagcg

SEQ ID NO: 28 - DNA, 20 bp, Artificial Sequence
  Description of Artificial Sequence: Synthetic oligonucleotide
tgctggccgggtccgggtcc

SEQ ID NO: 29 - DNA, 20 bp, Artificial Sequence
  Description of Artificial Sequence: Synthetic oligonucleotide
ccaggaccacccgcaccgcg

SEQ ID NO: 30 - DNA, 20 bp, Artificial Sequence
  Description of Artificial Sequence: Synthetic oligonucleotide
gcaatttatatttgagttca

SEQ ID NO: 31 - DNA, 20 bp, Artificial Sequence
  Description of Artificial Sequence: Synthetic oligonucleotide
gtgtgtagaaatcatatgtg

SEQ ID NO: 32 - DNA, 20 bp, Artificial Sequence
  Description of Artificial Sequence: Synthetic oligonucleotide
gggggactccggcagcccct

SEQ ID NO: 33 - DNA, 20 bp, Artificial Sequence
  Description of Artificial Sequence: Synthetic oligonucleotide
ccacccccgggacccggacc

SEQ ID NO: 34 - DNA, 20 bp, Artificial Sequence
  Description of Artificial Sequence: Synthetic oligonucleotide
gcacttccagaaaatgcagc

SEQ ID NO: 35 - DNA, 20 bp, Artificial Sequence
  Description of Artificial Sequence: Synthetic oligonucleotide
cccgggcagcccttctcagt

SEQ ID NO: 36 - DNA, 20 bp, Artificial Sequence
  Description of Artificial Sequence: Synthetic oligonucleotide
gagtcactaaaaagaaataa

SEQ ID NO: 37 - DNA, 20 bp, Artificial Sequence
  Description of Artificial Sequence: Synthetic oligonucleotide
gtgcccagacattaccttaa

SEQ ID NO: 38 - DNA, 20 bp, Artificial Sequence
  Description of Artificial Sequence: Synthetic oligonucleotide
catgtcggccgcgcactgcg

SEQ ID NO: 39 - DNA, 20 bp, Artificial Sequence
  Description of Artificial Sequence: Synthetic oligonucleotide
gtaagaatttaatgagcctc

SEQ ID NO: 40 - DNA, 20 bp, Artificial Sequence
  Description of Artificial Sequence: Synthetic oligonucleotide
ggatgattataattctctaa

SEQ ID NO: 41 - DNA, 20 bp, Artificial Sequence
  Description of Artificial Sequence: Synthetic oligonucleotide
gctggccgggtccgggtccc

SEQ ID NO: 42 - DNA, 20 bp, Artificial Sequence
  Description of Artificial Sequence: Synthetic oligonucleotide
gctcccgccgcgagaggtta

SEQ ID NO: 43 - DNA, 20 bp, Artificial Sequence
  Description of Artificial Sequence: Synthetic oligonucleotide
gtgagagaagaactctctct

SEQ ID NO: 44 - DNA, 20 bp, Artificial Sequence
  Description of Artificial Sequence: Synthetic oligonucleotide
cccaggcggcgtccctgagc

SEQ ID NO: 45 - DNA, 20 bp, Artificial Sequence
  Description of Artificial Sequence: Synthetic oligonucleotide
gttggggacatagtttgatt

SEQ ID NO: 46 - DNA, 20 bp, Artificial Sequence
  Description of Artificial Sequence: Synthetic oligonucleotide
cgggcggggcggggggcgca

SEQ ID NO: 47 - DNA, 20 bp, Artificial Sequence
  Description of Artificial Sequence: Synthetic oligonucleotide
aggccggcaggacacaggcg

SEQ ID NO: 48 - DNA, 20 bp, Artificial Sequence
  Description of Artificial Sequence: Synthetic oligonucleotide
cgaaacagacgccggcctgc

SEQ ID NO: 49 - DNA, 20 bp, Artificial Sequence
  Description of Artificial Sequence: Synthetic oligonucleotide
gtgctattcgcatatagggc

SEQ ID NO: 50 - DNA, 20 bp, Artificial Sequence
  Description of Artificial Sequence: Synthetic oligonucleotide
gtccaagagactcataaaga

SEQ ID NO: 51 - DNA, 20 bp, Artificial Sequence
  Description of Artificial Sequence: Synthetic oligonucleotide
tggagggcgctggccggccg

SEQ ID NO: 52 - DNA, 20 bp, Artificial Sequence
  Description of Artificial Sequence: Synthetic oligonucleotide
ggcgagcgcggccccacgcg

SEQ ID NO: 53 - DNA, 20 bp, Artificial Sequence
  Description of Artificial Sequence: Synthetic oligonucleotide
gacctttctttgaagaaaac

SEQ ID NO: 54 - DNA, 20 bp, Artificial Sequence
  Description of Artificial Sequence: Synthetic oligonucleotide
cgtgattctccaggtgccgc

SEQ ID NO: 55 - DNA, 20 bp, Artificial Sequence
  Description of Artificial Sequence: Synthetic oligonucleotide
gagcgtactatattcttaaa

SEQ ID NO: 56 - DNA, 20 bp, Artificial Sequence
  Description of Artificial Sequence: Synthetic oligonucleotide
gtgtccctgcagctgcgcgg

SEQ ID NO: 57 - DNA, 20 bp, Artificial Sequence
  Description of Artificial Sequence: Synthetic oligonucleotide
acagggctataagaggagcc

SEQ ID NO: 58 - DNA, 20 bp, Artificial Sequence
  Description of Artificial Sequence: Synthetic oligonucleotide
ggagaggaagtggagggcgc

SEQ ID NO: 59 - DNA, 20 bp, Artificial Sequence
  Description of Artificial Sequence: Synthetic oligonucleotide
gcccagaaggccccagccca

SEQ ID NO: 60 - DNA, 20 bp, Artificial Sequence
  Description of Artificial Sequence: Synthetic oligonucleotide
gacacgcgagtcggcggccg

SEQ ID NO: 61 - DNA, 20 bp, Artificial Sequence
  Description of Artificial Sequence: Synthetic oligonucleotide
gatggtgttcaagctttcta

SEQ ID NO: 62 - DNA, 20 bp, Artificial Sequence
  Description of Artificial Sequence: Synthetic oligonucleotide
ggggagcagagggacaccca

SEQ ID NO: 63 - DNA, 20 bp, Artificial Sequence
  Description of Artificial Sequence: Synthetic oligonucleotide
gcagaaacgtccgcgcggtg

SEQ ID NO: 64 - DNA, 20 bp, Artificial Sequence
  Description of Artificial Sequence: Synthetic oligonucleotide
gtggcccagctgccggctca

SEQ ID NO: 65 - DNA, 20 bp, Artificial Sequence
  Description of Artificial Sequence: Synthetic oligonucleotide
gtgggggttagacgaatatc

SEQ ID NO: 66 - DNA, 20 bp, Artificial Sequence
  Description of Artificial Sequence: Synthetic oligonucleotide
gaaaatggagtatagggttt

SEQ ID NO: 67 - DNA, 20 bp, Artificial Sequence
  Description of Artificial Sequence: Synthetic oligonucleotide
aaactcaccccccagcagca

SEQ ID NO: 68 - DNA, 20 bp, Artificial Sequence
  Description of Artificial Sequence: Synthetic oligonucleotide
gtccggggaggctgcgcctc

SEQ ID NO: 69 - DNA, 20 bp, Artificial Sequence
  Description of Artificial Sequence: Synthetic oligonucleotide
ggctataagaggagccgggc

SEQ ID NO: 70 - DNA, 20 bp, Artificial Sequence
  Description of Artificial Sequence: Synthetic oligonucleotide
tctcagtgggtcctgctggc

SEQ ID NO: 71 - DNA, 20 bp, Artificial Sequence
  Description of Artificial Sequence: Synthetic oligonucleotide
attctccaggtgccgccggg

SEQ ID NO: 72 - DNA, 20 bp, Artificial Sequence
  Description of Artificial Sequence: Synthetic oligonucleotide
acacgcgagtcggcggccga

SEQ ID NO: 73 - DNA, 20 bp, Artificial Sequence
  Description of Artificial Sequence: Synthetic oligonucleotide
gcccttctcctcatatggtt

SEQ ID NO: 74 - DNA, 20 bp, Artificial Sequence
  Description of Artificial Sequence: Synthetic oligonucleotide
gccggctcagggacgccgcc

SEQ ID NO: 75 - DNA, 20 bp, Artificial Sequence
  Description of Artificial Sequence: Synthetic oligonucleotide
tccggggaggctgcgcctca

SEQ ID NO: 76 - DNA, 20 bp, Artificial Sequence
  Description of Artificial Sequence: Synthetic oligonucleotide
gtggcctccgcgcagctgca

SEQ ID NO: 77 - DNA, 20 bp, Artificial Sequence
  Description of Artificial Sequence: Synthetic oligonucleotide
acgcgtggggccgcgctcgc

SEQ ID NO: 78 - DNA, 20 bp, Artificial Sequence
  Description of Artificial Sequence: Synthetic oligonucleotide
cagctgcagggacaccatga

SEQ ID NO: 79 - DNA, 20 bp, Artificial Sequence
  Description of Artificial Sequence: Synthetic oligonucleotide
ttctccaggtgccgccgggc

SEQ ID NO: 80 - DNA, 20 bp, Artificial Sequence
  Description of Artificial Sequence: Synthetic oligonucleotide
aggaagtggagggcgctggc

SEQ ID NO: 81 - DNA, 20 bp, Artificial Sequence
  Description of Artificial Sequence: Synthetic oligonucleotide
gaggccgttgcattgcccca

SEQ ID NO: 82 - DNA, 20 bp, Artificial Sequence
  Description of Artificial Sequence: Synthetic oligonucleotide
ggagccgggcgggcacggag

SEQ ID NO: 83 - DNA, 20 bp, Artificial Sequence
  Description of Artificial Sequence: Synthetic oligonucleotide
ggtaaagtcagataaaaata

SEQ ID NO: 84 - DNA, 20 bp, Artificial Sequence
  Description of Artificial Sequence: Synthetic oligonucleotide
ggtggcccagctgccggctc

SEQ ID NO: 85 - DNA, 20 bp, Artificial Sequence
  Description of Artificial Sequence: Synthetic oligonucleotide
ctcagtgggtcctgctggcc

SEQ ID NO: 86 - DNA, 20 bp, Artificial Sequence
  Description of Artificial Sequence: Synthetic oligonucleotide
tcagggtggcgccgcagaag

SEQ ID NO: 87 - DNA, 20 bp, Artificial Sequence
  Description of Artificial Sequence: Synthetic oligonucleotide
gggtcctgctggccgggtcc

SEQ ID NO: 88 - DNA, 20 bp, Artificial Sequence
  Description of Artificial Sequence: Synthetic oligonucleotide
ctgccggccttgctgctggg

SEQ ID NO: 89 - DNA, 20 bp, Artificial Sequence
  Description of Artificial Sequence: Synthetic oligonucleotide
ctcgtgaggggccggcaggc

SEQ ID NO: 90 - DNA, 20 bp, Artificial Sequence
  Description of Artificial Sequence: Synthetic oligonucleotide
ccgcgcggacgtttctgccg

SEQ ID NO: 91 - DNA, 20 bp, Artificial Sequence
  Description of Artificial Sequence: Synthetic oligonucleotide
gttataaaagtcttatggat

SEQ ID NO: 92 - DNA, 20 bp, Artificial Sequence
  Description of Artificial Sequence: Synthetic oligonucleotide
actgcaccccgttgcccagg

SEQ ID NO: 93 - DNA, 20 bp, Artificial Sequence
  Description of Artificial Sequence: Synthetic oligonucleotide
cagctgggccacctgcacgt

SEQ ID NO: 94 - DNA, 20 bp, Artificial Sequence
  Description of Artificial Sequence: Synthetic oligonucleotide
ggcagccccttggtctgcaa

SEQ ID NO: 95 - DNA, 20 bp, Artificial Sequence
  Description of Artificial Sequence: Synthetic oligonucleotide
gggctgccggagtccccctg

SEQ ID NO: 96 - DNA, 20 bp, Artificial Sequence
  Description of Artificial Sequence: Synthetic oligonucleotide
gtgactttatcctaaaggaa

SEQ ID NO: 97 - DNA, 20 bp, Artificial Sequence
  Description of Artificial Sequence: Synthetic oligonucleotide
ggcacctttacattatgtaa

SEQ ID NO: 98 - DNA, 20 bp, Artificial Sequence
  Description of Artificial Sequence: Synthetic oligonucleotide
gtttccaataaatagtcacg

SEQ ID NO: 99 - DNA, 20 bp, Artificial Sequence
  Description of Artificial Sequence: Synthetic oligonucleotide
ctgccggagtccccctgtgg

SEQ ID NO: 100 - DNA, 20 bp, Artificial Sequence
  Description of Artificial Sequence: Synthetic oligonucleotide
ggctttggagcccaagtgag

SEQ ID NO: 101 - DNA, 20 bp, Artificial Sequence
  Description of Artificial Sequence: Synthetic oligonucleotide
gccccagttatgtcttatct

SEQ ID NO: 102 - DNA, 20 bp, Artificial Sequence
  Description of Artificial Sequence: Synthetic oligonucleotide
cacggcgaacacctgccggg

SEQ ID NO: 103 - DNA, 20 bp, Artificial Sequence
  Description of Artificial Sequence: Synthetic oligonucleotide
aggagccgggcgggcacgga

SEQ ID NO: 104 - DNA, 20 bp, Artificial Sequence
  Description of Artificial Sequence: Synthetic oligonucleotide
gaatatatttagctactttc

SEQ ID NO: 105 - DNA, 20 bp, Artificial Sequence
  Description of Artificial Sequence: Synthetic oligonucleotide
gacttgacttataagaggtg

SEQ ID NO: 106 - DNA, 20 bp, Artificial Sequence
  Description of Artificial Sequence: Synthetic oligonucleotide
atggtgtccctgcagctgcg

SEQ ID NO: 107 - DNA, 20 bp, Artificial Sequence
  Description of Artificial Sequence: Synthetic oligonucleotide
gaattcagtattaaacaaca

SEQ ID NO: 108 - DNA, 20 bp, Artificial Sequence
  Description of Artificial Sequence: Synthetic oligonucleotide
ggagggcgctggccggccgt

SEQ ID NO: 109 - DNA, 20 bp, Artificial Sequence
  Description of Artificial Sequence: Synthetic oligonucleotide
ccagcaggacccactgagaa

SEQ ID NO: 110 - DNA, 20 bp, Artificial Sequence
  Description of Artificial Sequence: Synthetic oligonucleotide
gatgctaaaaatctaaacct

SEQ ID NO: 111 - DNA, 20 bp, Artificial Sequence
  Description of Artificial Sequence: Synthetic oligonucleotide
cgcgctggcctcggagattg

SEQ ID NO: 112 - DNA, 20 bp, Artificial Sequence
  Description of Artificial Sequence: Synthetic oligonucleotide
gaaggccccagcccatggcc

SEQ ID NO: 113 - DNA, 20 bp, Artificial Sequence
  Description of Artificial Sequence: Synthetic oligonucleotide
ggccacagtgagatgcagga

SEQ ID NO: 114 - DNA, 20 bp, Artificial Sequence
  Description of Artificial Sequence: Synthetic oligonucleotide
gcaggacgctggcgatccca

SEQ ID NO: 115 - DNA, 20 bp, Artificial Sequence
  Description of Artificial Sequence: Synthetic oligonucleotide
gtcctgccggccttgctgct

SEQ ID NO: 116 - DNA, 20 bp, Artificial Sequence
  Description of Artificial Sequence: Synthetic oligonucleotide
ctcccgccgcgagaggttat

SEQ ID NO: 117 - DNA, 20 bp, Artificial Sequence
  Description of Artificial Sequence: Synthetic oligonucleotide
gagcccataacctctcgcgg

SEQ ID NO: 118 - DNA, 20 bp, Artificial Sequence
  Description of Artificial Sequence: Synthetic oligonucleotide
gacgaagttgggcgcaatca

SEQ ID NO: 119 - DNA, 20 bp, Artificial Sequence
  Description of Artificial Sequence: Synthetic oligonucleotide
gagacattagttaggaaata

SEQ ID NO: 120 - DNA, 20 bp, Artificial Sequence
  Description of Artificial Sequence: Synthetic oligonucleotide
ggcactgcaccccgttgccc

SEQ ID NO: 121 - DNA, 20 bp, Artificial Sequence
  Description of Artificial Sequence: Synthetic oligonucleotide
ccaccggggcaaaggcatcg

SEQ ID NO: 122 - DNA, 20 bp, Artificial Sequence
  Description of Artificial Sequence: Synthetic oligonucleotide
cccgcacccggtgtgtcccc

SEQ ID NO: 123 - DNA, 20 bp, Artificial Sequence
  Description of Artificial Sequence: Synthetic oligonucleotide
gtggggacaggggttgtcct

SEQ ID NO: 124 - DNA, 20 bp, Artificial Sequence
  Description of Artificial Sequence: Synthetic oligonucleotide
gcctattccgttcattcaat

SEQ ID NO: 125 - DNA, 20 bp, Artificial Sequence
  Description of Artificial Sequence: Synthetic oligonucleotide
gtaagcttaaaacattagta

SEQ ID NO: 126 - DNA, 20 bp, Artificial Sequence
  Description of Artificial Sequence: Synthetic oligonucleotide
gctcctcacagaattggcta

SEQ ID NO: 127 - DNA, 20 bp, Artificial Sequence
  Description of Artificial Sequence: Synthetic oligonucleotide
gcagccaattattgccaaat

SEQ ID NO: 128 - DNA, 20 bp, Artificial Sequence
  Description of Artificial Sequence: Synthetic oligonucleotide
ggaaacggaggaagtccact

SEQ ID NO: 129 - DNA, 20 bp, Artificial Sequence
  Description of Artificial Sequence: Synthetic oligonucleotide
aaacgtccgcgcggtgcggg

SEQ ID NO: 130 - DNA, 20 bp, Artificial Sequence
  Description of Artificial Sequence: Synthetic oligonucleotide
cccccagcagcaaggccggc

SEQ ID NO: 131 - DNA, 20 bp, Artificial Sequence
  Description of Artificial Sequence: Synthetic oligonucleotide
cccaggcaccgcgctggcct

SEQ ID NO: 132 - DNA, 20 bp, Artificial Sequence
  Description of Artificial Sequence: Synthetic oligonucleotide
gaatccacacataaaagcac

SEQ ID NO: 133 - DNA, 20 bp, Artificial Sequence
  Description of Artificial Sequence: Synthetic oligonucleotide
ggtggttagcccagttcatt

SEQ ID NO: 134 - DNA, 20 bp, Artificial Sequence
  Description of Artificial Sequence: Synthetic oligonucleotide
tcttatagccctgtgctggg

SEQ ID NO: 135 - DNA, 20 bp, Artificial Sequence
  Description of Artificial Sequence: Synthetic oligonucleotide
ggccttctgggcaggaaccg

SEQ ID NO: 136 - DNA, 20 bp, Artificial Sequence
  Description of Artificial Sequence: Synthetic oligonucleotide
gctcattcgaacttcttcca

SEQ ID NO: 137 - DNA, 20 bp, Artificial Sequence
  Description of Artificial Sequence: Synthetic oligonucleotide
agcccataacctctcgcggc

SEQ ID NO: 138 - DNA, 20 bp, Artificial Sequence
  Description of Artificial Sequence: Synthetic oligonucleotide
ggccatgggctggggccttc

SEQ ID NO: 139 - DNA, 20 bp, Artificial Sequence
  Description of Artificial Sequence: Synthetic oligonucleotide
tgtgggcagctgaggtgacc

SEQ ID NO: 140 - DNA, 20 bp, Artificial Sequence
  Description of Artificial Sequence: Synthetic oligonucleotide
gttagaatccataacttctc

SEQ ID NO: 141 - DNA, 20 bp, Artificial Sequence
  Description of Artificial Sequence: Synthetic oligonucleotide
cctcggagattgtggggggc

SEQ ID NO: 142 - DNA, 20 bp, Artificial Sequence
  Description of Artificial Sequence: Synthetic oligonucleotide
acggcgaacacctgccgggt

SEQ ID NO: 143 - DNA, 20 bp, Artificial Sequence
  Description of Artificial Sequence: Synthetic oligonucleotide
tgggtcctgctggccgggtc

SEQ ID NO: 144 - DNA, 20 bp, Artificial Sequence
  Description of Artificial Sequence: Synthetic oligonucleotide
gtctctagatagtgtaaatg

SEQ ID NO: 145 - DNA, 20 bp, Artificial Sequence
  Description of Artificial Sequence: Synthetic oligonucleotide
gtttgtaacttatggtactg

SEQ ID NO: 146 - DNA, 20 bp, Artificial Sequence
  Description of Artificial Sequence: Synthetic oligonucleotide
gtgattctccaggtgccgcc

SEQ ID NO: 147 - DNA, 20 bp, Artificial Sequence
  Description of Artificial Sequence: Synthetic oligonucleotide
gtagacacaaacttgtgtaa

SEQ ID NO: 148 - DNA, 20 bp, Artificial Sequence
  Description of Artificial Sequence: Synthetic oligonucleotide
cctgccggccttgctgctgg

SEQ ID NO: 149 - DNA, 20 bp, Artificial Sequence
  Description of Artificial Sequence: Synthetic oligonucleotide
ccggccccccacaatctccg

SEQ ID NO: 150 - DNA, 20 bp, Artificial Sequence
  Description of Artificial Sequence: Synthetic oligonucleotide
gacaacctcacagaaaataa

SEQ ID NO: 151 - DNA, 20 bp, Artificial Sequence
  Description of Artificial Sequence: Synthetic oligonucleotide
tcatggtggggctggggctc

SEQ ID NO: 152 - DNA, 20 bp, Artificial Sequence
  Description of Artificial Sequence: Synthetic oligonucleotide
gagcacttaatatgtgcaaa

SEQ ID NO: 153 - DNA, 20 bp, Artificial Sequence
  Description of Artificial Sequence: Synthetic oligonucleotide
ctgcacggcgaacacctgcc

SEQ ID NO: 154 - DNA, 20 bp, Artificial Sequence
  Description of Artificial Sequence: Synthetic oligonucleotide
cacaatctccgaggccagcg

SEQ ID NO: 155 - DNA, 20 bp, Artificial Sequence
  Description of Artificial Sequence: Synthetic oligonucleotide
gatcaatacttcacagtcct

SEQ ID NO: 156 - DNA, 20 bp, Artificial Sequence
  Description of Artificial Sequence: Synthetic oligonucleotide
gcactaacattcataaatat

SEQ ID NO: 157 - DNA, 20 bp, Artificial Sequence
  Description of Artificial Sequence: Synthetic oligonucleotide
ggttgtgaatacatagtaat

SEQ ID NO: 158 - DNA, 20 bp, Artificial Sequence
  Description of Artificial Sequence: Synthetic oligonucleotide
gaaagttaatatctttagct

SEQ ID NO: 159 - DNA, 20 bp, Artificial Sequence
  Description of Artificial Sequence: Synthetic oligonucleotide
agtggcctccgcgcagctgc

SEQ ID NO: 160 - DNA, 20 bp, Artificial Sequence
  Description of Artificial Sequence: Synthetic oligonucleotide
cggggtgcagtgcctggcca

SEQ ID NO: 161 - DNA, 20 bp, Artificial Sequence
  Description of Artificial Sequence: Synthetic oligonucleotide
gcgtccctgagccggcagct

SEQ ID NO: 162 - DNA, 20 bp, Artificial Sequence
  Description of Artificial Sequence: Synthetic oligonucleotide
tgggctggggccttctgggc

SEQ ID NO: 163 - DNA, 20 bp, Artificial Sequence
  Description of Artificial Sequence: Synthetic oligonucleotide
ctgcgcggaggccacttctg

SEQ ID NO: 164 - DNA, 20 bp, Artificial Sequence
  Description of Artificial Sequence: Synthetic oligonucleotide
gcagccccttggtctgcaac

SEQ ID NO: 165 - DNA, 20 bp, Artificial Sequence
  Description of Artificial Sequence: Synthetic oligonucleotide
gggcaacggggtgcagtgcc

SEQ ID NO: 166 - DNA, 20 bp, Artificial Sequence
  Description of Artificial Sequence: Synthetic oligonucleotide
gacatttgacactgaagcta

SEQ ID NO: 167 - DNA, 20 bp, Artificial Sequence
  Description of Artificial Sequence: Synthetic oligonucleotide
ccgcgcggtgcgggtggtcc

SEQ ID NO: 168 - DNA, 20 bp, Artificial Sequence
  Description of Artificial Sequence: Synthetic oligonucleotide
gaattaaattcactttgaac

SEQ ID NO: 169 - DNA, 20 bp, Artificial Sequence
  Description of Artificial Sequence: Synthetic oligonucleotide
gtacttccactgtcactttg

SEQ ID NO: 170 - DNA, 20 bp, Artificial Sequence
  Description of Artificial Sequence: Synthetic oligonucleotide
gcgctggcctcggagattgt

SEQ ID NO: 171 - DNA, 20 bp, Artificial Sequence
  Description of Artificial Sequence: Synthetic oligonucleotide
gtgtaaagcttccacttcac

SEQ ID NO: 172 - DNA, 20 bp, Artificial Sequence
  Description of Artificial Sequence: Synthetic oligonucleotide
gtttatttgcaaggaataca

SEQ ID NO: 173 - DNA, 20 bp, Artificial Sequence
  Description of Artificial Sequence: Synthetic oligonucleotide
tgtgtccccaggcaccgcgc

SEQ ID NO: 174 - DNA, 20 bp, Artificial Sequence
  Description of Artificial Sequence: Synthetic oligonucleotide
gggacgccgcctgggcaacg

SEQ ID NO: 175 - DNA, 20 bp, Artificial Sequence
  Description of Artificial Sequence: Synthetic oligonucleotide
gctctacataggtaacagga

SEQ ID NO: 176 - DNA, 20 bp, Artificial Sequence
  Description of Artificial Sequence: Synthetic oligonucleotide
ggcgtccctgagccggcagc

SEQ ID NO: 177 - DNA, 20 bp, Artificial Sequence
  Description of Artificial Sequence: Synthetic oligonucleotide
gtcatcagaagaaacctctt

SEQ ID NO: 178 - DNA, 20 bp, Artificial Sequence
  Description of Artificial Sequence: Synthetic oligonucleotide
gggctataagaggagccggg

SEQ ID NO: 179 - DNA, 20 bp, Artificial Sequence
  Description of Artificial Sequence: Synthetic oligonucleotide
catggtggggctggggctcc

SEQ ID NO: 180 - DNA, 20 bp, Artificial Sequence
  Description of Artificial Sequence: Synthetic oligonucleotide
cagaaacgtccgcgcggtgc

SEQ ID NO: 181 - DNA, 20 bp, Artificial Sequence
  Description of Artificial Sequence: Synthetic oligonucleotide
gttcaaacaacatactgaaa

SEQ ID NO: 182 - DNA, 20 bp, Artificial Sequence
  Description of Artificial Sequence: Synthetic oligonucleotide
gaattgaggcagtagcctct

SEQ ID NO: 183 - DNA, 20 bp, Artificial Sequence
  Description of Artificial Sequence: Synthetic oligonucleotide
gaggggaccctggtttgagg

SEQ ID NO: 184 - DNA, 20 bp, Artificial Sequence
  Description of Artificial Sequence: Synthetic oligonucleotide
gtgagtttctttctgccagg

SEQ ID NO: 185 - DNA, 20 bp, Artificial Sequence
  Description of Artificial Sequence: Synthetic oligonucleotide
cacagggctataagaggagc

SEQ ID NO: 186 - DNA, 20 bp, Artificial Sequence
  Description of Artificial Sequence: Synthetic oligonucleotide
tgggatcgccagcgtcctgc

SEQ ID NO: 187 - DNA, 20 bp, Artificial Sequence
  Description of Artificial Sequence: Synthetic oligonucleotide
gtgagctcctcattcattcc

SEQ ID NO: 188 - DNA, 20 bp, Artificial Sequence
  Description of Artificial Sequence: Synthetic oligonucleotide
caccttgtctgcctccacag

SEQ ID NO: 189 - DNA, 20 bp, Artificial Sequence
  Description of Artificial Sequence: Synthetic oligonucleotide
cgcgcggtgcgggtggtcct

SEQ ID NO: 190 - DNA, 20 bp, Artificial Sequence
  Description of Artificial Sequence: Synthetic oligonucleotide
gtgcagcgcatcttcgaaaa

SEQ ID NO: 191 - DNA, 20 bp, Artificial Sequence
  Description of Artificial Sequence: Synthetic oligonucleotide
gtgttctcagttatatgcaa

SEQ ID NO: 192 - DNA, 20 bp, Artificial Sequence
  Description of Artificial Sequence: Synthetic oligonucleotide
cactctcgtgaggggccggc

SEQ ID NO: 193 - DNA, 20 bp, Artificial Sequence
  Description of Artificial Sequence: Synthetic oligonucleotide
ggccacattaataaccttac

SEQ ID NO: 194 - DNA, 20 bp, Artificial Sequence
  Description of Artificial Sequence: Synthetic oligonucleotide
gtaaaaatgtccttgtcacc

SEQ ID NO: 195 - DNA, 20 bp, Artificial Sequence
  Description of Artificial Sequence: Synthetic oligonucleotide
gttactgaagtaacttgcct

SEQ ID NO: 196 - DNA, 20 bp, Artificial Sequence
  Description of Artificial Sequence: Synthetic oligonucleotide
ggtgtgacaaggagaacaaa

SEQ ID NO: 197 - DNA, 20 bp, Artificial Sequence
  Description of Artificial Sequence: Synthetic oligonucleotide
gctgcacggcgaacacctgc

SEQ ID NO: 198 - DNA, 20 bp, Artificial Sequence
  Description of Artificial Sequence: Synthetic oligonucleotide
gaagttgggcgcaatcaggg

SEQ ID NO: 199 - DNA, 20 bp, Artificial Sequence
  Description of Artificial Sequence: Synthetic oligonucleotide
tctgcccctccgtgcccgcc

SEQ ID NO: 200 - DNA, 20 bp, Artificial Sequence
  Description of Artificial Sequence: Synthetic oligonucleotide
gatgctggagagtgtgggtg

SEQ ID NO: 201 - DNA, 20 bp, Artificial Sequence
  Description of Artificial Sequence: Synthetic oligonucleotide
gtgcactacaagtaatacta

SEQ ID NO: 202 - DNA, 20 bp, Artificial Sequence
  Description of Artificial Sequence: Synthetic oligonucleotide
gagggcgctggccggccgtg

SEQ ID NO: 203 - DNA, 20 bp, Artificial Sequence
  Description of Artificial Sequence: Synthetic oligonucleotide
ccgagggtcatggtggggct

SEQ ID NO: 204 - DNA, 20 bp, Artificial Sequence
  Description of Artificial Sequence: Synthetic oligonucleotide
cgtctgtttcgtacgtgccc

SEQ ID NO: 205 - DNA, 20 bp, Artificial Sequence
  Description of Artificial Sequence: Synthetic oligonucleotide
tgacgaagttgggcgcaatc

SEQ ID NO: 206 - DNA, 20 bp, Artificial Sequence
  Description of Artificial Sequence: Synthetic oligonucleotide
tcaccccccagcagcaaggc

SEQ ID NO: 207 - DNA, 20 bp, Artificial Sequence
  Description of Artificial Sequence: Synthetic oligonucleotide
ccggctcagggacgccgcct

SEQ ID NO: 208 - DNA, 20 bp, Artificial Sequence
  Description of Artificial Sequence: Synthetic oligonucleotide
gtggaatcattactccaaaa

SEQ ID NO: 209 - DNA, 20 bp, Artificial Sequence
  Description of Artificial Sequence: Synthetic oligonucleotide
gaaacaagctaatattatca

SEQ ID NO: 210 - DNA, 20 bp, Artificial Sequence
  Description of Artificial Sequence: Synthetic oligonucleotide
cagggacgccgcctgggcaa

SEQ ID NO: 211 - DNA, 20 bp, Artificial Sequence
  Description of Artificial Sequence: Synthetic oligonucleotide
cacccacactctccagcatc

SEQ ID NO: 212 - DNA, 20 bp, Artificial Sequence
  Description of Artificial Sequence: Synthetic oligonucleotide
ctggcctcggagattgtggg

SEQ ID NO: 213 - DNA, 20 bp, Artificial Sequence
  Description of Artificial Sequence: Synthetic oligonucleotide
gcggccgacatgacgaagtt

SEQ ID NO: 214 - DNA, 20 bp, Artificial Sequence
  Description of Artificial Sequence: Synthetic oligonucleotide
tttcatcaacgccctgtgcc

SEQ ID NO: 215 - DNA, 20 bp, Artificial Sequence
  Description of Artificial Sequence: Synthetic oligonucleotide
gtatcgtaaatggatgtaga

SEQ ID NO: 216 - DNA, 20 bp, Artificial Sequence
  Description of Artificial Sequence: Synthetic oligonucleotide
gaggagccgggcgggcacgg

SEQ ID NO: 217 - DNA, 20 bp, Artificial Sequence
  Description of Artificial Sequence: Synthetic oligonucleotide
ggaaaagacacgcgagtcgg

SEQ ID NO: 218 - DNA, 20 bp, Artificial Sequence
  Description of Artificial Sequence: Synthetic oligonucleotide
ccgaattggagcgcttcaac

SEQ ID NO: 219 - DNA, 20 bp, Artificial Sequence
  Description of Artificial Sequence: Synthetic oligonucleotide
gaagcaggaggtgtaccagt

SEQ ID NO: 220 - DNA, 20 bp, Artificial Sequence
  Description of Artificial Sequence: Synthetic oligonucleotide
cacattcgccacgcagtgcg

SEQ ID NO: 221 - DNA, 20 bp, Artificial Sequence
  Description of Artificial Sequence: Synthetic oligonucleotide
acttcctctcccctggcaca

SEQ ID NO: 222 - DNA, 20 bp, Artificial Sequence
  Description of Artificial Sequence: Synthetic oligonucleotide
atgctggagagtgtgggtgt

SEQ ID NO: 223 - DNA, 20 bp, Artificial Sequence
  Description of Artificial Sequence: Synthetic oligonucleotide
gctggcctcggagattgtgg

SEQ ID NO: 224 - DNA, 20 bp, Artificial Sequence
  Description of Artificial Sequence: Synthetic oligonucleotide
gctgcatgctggccagcagc

SEQ ID NO: 225 - DNA, 20 bp, Artificial Sequence
  Description of Artificial Sequence: Synthetic oligonucleotide
aacgccctgtgccaggggag

SEQ ID NO: 226 - DNA, 20 bp, Artificial Sequence
  Description of Artificial Sequence: Synthetic oligonucleotide
ctcctcttatagccctgtgc

SEQ ID NO: 227 - DNA, 20 bp, Artificial Sequence
  Description of Artificial Sequence: Synthetic oligonucleotide
gccttctgggcaggaaccgt

SEQ ID NO: 228 - DNA, 20 bp, Artificial Sequence
  Description of Artificial Sequence: Synthetic oligonucleotide
gtgggcagctgaggtgaccc

SEQ ID NO: 229 - DNA, 20 bp, Artificial Sequence
  Description of Artificial Sequence: Synthetic oligonucleotide
tgcagtgcctggccatgggc

SEQ ID NO: 230 - DNA, 20 bp, Artificial Sequence
  Description of Artificial Sequence: Synthetic oligonucleotide
agctgcagggacaccatgaa

SEQ ID NO: 231 - DNA, 20 bp, Artificial Sequence
  Description of Artificial Sequence: Synthetic oligonucleotide
gacgtgagctacaatgggaa

SEQ ID NO: 232 - DNA, 20 bp, Artificial Sequence
  Description of Artificial Sequence: Synthetic oligonucleotide
gccctgaggcgcagcctccc

SEQ ID NO: 233 - DNA, 20 bp, Artificial Sequence
  Description of Artificial Sequence: Synthetic oligonucleotide
gagtcggcggccgagggtca

SEQ ID NO: 234 - DNA, 20 bp, Artificial Sequence
  Description of Artificial Sequence: Synthetic oligonucleotide
tgggagcccataacctctcg

SEQ ID NO: 235 - DNA, 20 bp, Artificial Sequence
  Description of Artificial Sequence: Synthetic oligonucleotide
tcatcaacgccctgtgccag

SEQ ID NO: 236 - DNA, 20 bp, Artificial Sequence
  Description of Artificial Sequence: Synthetic oligonucleotide
gcgagaggttatgggctccc

SEQ ID NO: 237 - DNA, 20 bp, Artificial Sequence
  Description of Artificial Sequence: Synthetic oligonucleotide
gtgccagatgctggagagtg

SEQ ID NO: 238 - DNA, 20 bp, Artificial Sequence
  Description of Artificial Sequence: Synthetic oligonucleotide
cgagggtcatggtggggctg

SEQ ID NO: 239 - DNA, 20 bp, Artificial Sequence
  Description of Artificial Sequence: Synthetic oligonucleotide
gatcacaaactcatctttca

SEQ ID NO: 240 - DNA, 20 bp, Artificial Sequence
  Description of Artificial Sequence: Synthetic oligonucleotide
gccatgggctggggccttct

SEQ ID NO: 241 - DNA, 20 bp, Artificial Sequence
  Description of Artificial Sequence: Synthetic oligonucleotide
ggaaaacactgttcaactta

SEQ ID NO: 242 - DNA, 20 bp, Artificial Sequence
  Description of Artificial Sequence: Synthetic oligonucleotide
acccgggcagcccttctcag

SEQ ID NO: 243 - DNA, 20 bp, Artificial Sequence
  Description of Artificial Sequence: Synthetic oligonucleotide
gcagggaatggaaatgaata

SEQ ID NO: 244 - DNA, 20 bp, Artificial Sequence
  Description of Artificial Sequence: Synthetic oligonucleotide
ggggtgcagtgcctggccat

SEQ ID NO: 245 - DNA, 20 bp, Artificial Sequence
  Description of Artificial Sequence: Synthetic oligonucleotide
gcatagttagtatgggactt

SEQ ID NO: 246 - DNA, 20 bp, Artificial Sequence
  Description of Artificial Sequence: Synthetic oligonucleotide
gttttcgaagatgcgctgca

SEQ ID NO: 247 - DNA, 20 bp, Artificial Sequence
  Description of Artificial Sequence: Synthetic oligonucleotide
gaaaatcttacacatggata

SEQ ID NO: 248 - DNA, 20 bp, Artificial Sequence
  Description of Artificial Sequence: Synthetic oligonucleotide
ggacataagaagttgatagt

SEQ ID NO: 249 - DNA, 20 bp, Artificial Sequence
  Description of Artificial Sequence: Synthetic oligonucleotide
gcaggagctcaacgtgacgg

SEQ ID NO: 250 - DNA, 20 bp, Artificial Sequence
  Description of Artificial Sequence: Synthetic oligonucleotide
accgcgcggacgtttctgcc

SEQ ID NO: 251 - DNA, 20 bp, Artificial Sequence
  Description of Artificial Sequence: Synthetic oligonucleotide
gttaaattatggtgattcta

SEQ ID NO: 252 - DNA, 20 bp, Artificial Sequence
  Description of Artificial Sequence: Synthetic oligonucleotide
gtctgtttcgtacgtgccct

SEQ ID NO: 253 - DNA, 20 bp, Artificial Sequence
  Description of Artificial Sequence: Synthetic oligonucleotide
gtctttcacgatgtacccta

SEQ ID NO: 254 - DNA, 20 bp, Artificial Sequence
  Description of Artificial Sequence: Synthetic oligonucleotide
gaaataacatacgactatac

SEQ ID NO: 255 - DNA, 20 bp, Artificial Sequence
  Description of Artificial Sequence: Synthetic oligonucleotide
ccgtcacgttgagctcctgc

SEQ ID NO: 256 - DNA, 20 bp, Artificial Sequence
  Description of Artificial Sequence: Synthetic oligonucleotide
ggaggatcacagcaagcttc

SEQ ID NO: 257 - DNA, 20 bp, Artificial Sequence
  Description of Artificial Sequence: Synthetic oligonucleotide
tgtcgttgagcaagtttacg

SEQ ID NO: 258 - DNA, 20 bp, Artificial Sequence
  Description of Artificial Sequence: Synthetic oligonucleotide
ccaccttgtctgcctccaca

SEQ ID NO: 259 - DNA, 20 bp, Artificial Sequence
  Description of Artificial Sequence: Synthetic oligonucleotide
gcatcatagcaagaagagtt

SEQ ID NO: 260 - DNA, 20 bp, Artificial Sequence
  Description of Artificial Sequence: Synthetic oligonucleotide
cccttctcagtgggtcctgc

SEQ ID NO: 261 - DNA, 20 bp, Artificial Sequence
  Description of Artificial Sequence: Synthetic oligonucleotide
ctctatcatccaacgctccg

SEQ ID NO: 262 - DNA, 20 bp, Artificial Sequence
  Description of Artificial Sequence: Synthetic oligonucleotide
accatgaagggccacgcgtg

SEQ ID NO: 263 - DNA, 20 bp, Artificial Sequence
  Description of Artificial Sequence: Synthetic oligonucleotide
ttcatcaacgccctgtgcca

SEQ ID NO: 264 - DNA, 20 bp, Artificial Sequence
  Description of Artificial Sequence: Synthetic oligonucleotide
caccgcgcggacgtttctgc

SEQ ID NO: 265 - DNA, 20 bp, Artificial Sequence
  Description of Artificial Sequence: Synthetic oligonucleotide
aaactgtgccaccggggcaa

SEQ ID NO: 266 - DNA, 20 bp, Artificial Sequence
  Description of Artificial Sequence: Synthetic oligonucleotide
cctgcaggagctcaacgtga

SEQ ID NO: 267 - DNA, 20 bp, Artificial Sequence
  Description of Artificial Sequence: Synthetic oligonucleotide
gttgcagaccaaggggctgc

SEQ ID NO: 268 - DNA, 20 bp, Artificial Sequence
  Description of Artificial Sequence: Synthetic oligonucleotide
gaagataatgtctgagtgat

SEQ ID NO: 269 - DNA, 20 bp, Artificial Sequence
  Description of Artificial Sequence: Synthetic oligonucleotide
gcacggaggggcagagaccc

SEQ ID NO: 270 - DNA, 20 bp, Artificial Sequence
  Description of Artificial Sequence: Synthetic oligonucleotide
acccactgagaagggctgcc

SEQ ID NO: 271 - DNA, 20 bp, Artificial Sequence
  Description of Artificial Sequence: Synthetic oligonucleotide
cgaggccagcgcggtgcctg

SEQ ID NO: 272 - DNA, 20 bp, Artificial Sequence
  Description of Artificial Sequence: Synthetic oligonucleotide
ggcggccgagggtcatggtg

SEQ ID NO: 273 - DNA, 20 bp, Artificial Sequence
  Description of Artificial Sequence: Synthetic oligonucleotide
ccccggcagaaacgtccgcg

SEQ ID NO: 274 - DNA, 20 bp, Artificial Sequence
  Description of Artificial Sequence: Synthetic oligonucleotide
tcggcggccgagggtcatgg

SEQ ID NO: 275 - DNA, 20 bp, Artificial Sequence
  Description of Artificial Sequence: Synthetic oligonucleotide
gccagcaggacccactgaga

SEQ ID NO: 276 - DNA, 20 bp, Artificial Sequence
  Description of Artificial Sequence: Synthetic oligonucleotide
agttcttaggtaccccacgt

SEQ ID NO: 277 - DNA, 20 bp, Artificial Sequence
  Description of Artificial Sequence: Synthetic oligonucleotide
ccggccgtggggcaatgcaa

SEQ ID NO: 278 - DNA, 20 bp, Artificial Sequence
  Description of Artificial Sequence: Synthetic oligonucleotide
tcctgccggccttgctgctg

SEQ ID NO: 279 - DNA, 20 bp, Artificial Sequence
  Description of Artificial Sequence: Synthetic oligonucleotide
atgtttattgtgccagatgc

SEQ ID NO: 280 - DNA, 20 bp, Artificial Sequence
  Description of Artificial Sequence: Synthetic oligonucleotide
atgcaacggcctcccagcac

SEQ ID NO: 281 - DNA, 20 bp, Artificial Sequence
  Description of Artificial Sequence: Synthetic oligonucleotide
tctgcactctcgtgaggggc

SEQ ID NO: 282 - DNA, 20 bp, Artificial Sequence
  Description of Artificial Sequence: Synthetic oligonucleotide
tcctcttatagccctgtgct

SEQ ID NO: 283 - DNA, 20 bp, Artificial Sequence
  Description of Artificial Sequence: Synthetic oligonucleotide
gacgttgctgcgacggcaga

SEQ ID NO: 284 - DNA, 20 bp, Artificial Sequence
  Description of Artificial Sequence: Synthetic oligonucleotide
ggcacgtacgaaacagacgc

SEQ ID NO: 285 - DNA, 20 bp, Artificial Sequence
  Description of Artificial Sequence: Synthetic oligonucleotide
gatcaactctcatcatatac

SEQ ID NO: 286 - DNA, 20 bp, Artificial Sequence
  Description of Artificial Sequence: Synthetic oligonucleotide
ccgaggccagcgcggtgcct

SEQ ID NO: 287 - DNA, 20 bp, Artificial Sequence
  Description of Artificial Sequence: Synthetic oligonucleotide
cacttcctctcccctggcac

SEQ ID NO: 288 - DNA, 20 bp, Artificial Sequence
  Description of Artificial Sequence: Synthetic oligonucleotide
agggacgccgcctgggcaac

SEQ ID NO: 289 - DNA, 20 bp, Artificial Sequence
  Description of Artificial Sequence: Synthetic oligonucleotide
gtggccgacccgttgagctg

SEQ ID NO: 290 - DNA, 20 bp, Artificial Sequence
  Description of Artificial Sequence: Synthetic oligonucleotide
cggcggccgagggtcatggt

SEQ ID NO: 291 - DNA, 20 bp, Artificial Sequence
  Description of Artificial Sequence: Synthetic oligonucleotide
gcagcctccccggacgaagg

SEQ ID NO: 292 - DNA, 20 bp, Artificial Sequence
  Description of Artificial Sequence: Synthetic oligonucleotide
aggggttgtcctcggagcgt

SEQ ID NO: 293 - DNA, 20 bp, Artificial Sequence
  Description of Artificial Sequence: Synthetic oligonucleotide
gttgagctcctgcaggacgc

SEQ ID NO: 294 - DNA, 20 bp, Artificial Sequence
  Description of Artificial Sequence: Synthetic oligonucleotide
gccccacgcgtggcccttca

SEQ ID NO: 295 - DNA, 20 bp, Artificial Sequence
  Description of Artificial Sequence: Synthetic oligonucleotide
ccccgatgcctttgccccgg

SEQ ID NO: 296 - DNA, 20 bp, Artificial Sequence
  Description of Artificial Sequence: Synthetic oligonucleotide
gccgagggtcatggtggggc

SEQ ID NO: 297 - DNA, 20 bp, Artificial Sequence
  Description of Artificial Sequence: Synthetic oligonucleotide
acaacccctgtccccacccc

SEQ ID NO: 298 - DNA, 20 bp, Artificial Sequence
  Description of Artificial Sequence: Synthetic oligonucleotide
ggaattgcctccttcgtccg

SEQ ID NO: 299 - DNA, 20 bp, Artificial Sequence
  Description of Artificial Sequence: Synthetic oligonucleotide
cgtgcaggtggcccagctgc

SEQ ID NO: 300 - DNA, 20 bp, Artificial Sequence
  Description of Artificial Sequence: Synthetic oligonucleotide
caacgacatcgtgattctcc

SEQ ID NO: 301 - DNA, 20 bp, Artificial Sequence
  Description of Artificial Sequence: Synthetic oligonucleotide
ggaagtgagttaaaagaagt

SEQ ID NO: 302 - DNA, 20 bp, Artificial Sequence
  Description of Artificial Sequence: Synthetic oligonucleotide
tcccacggttcctgcccaga

SEQ ID NO: 303 - DNA, 20 bp, Artificial Sequence
  Description of Artificial Sequence: Synthetic oligonucleotide
gccgacccgttgagctgtgg

SEQ ID NO: 304 - DNA, 20 bp, Artificial Sequence
  Description of Artificial Sequence: Synthetic oligonucleotide
gcagtgcctggccatgggct

SEQ ID NO: 305 - DNA, 20 bp, Artificial Sequence
  Description of Artificial Sequence: Synthetic oligonucleotide
cgcggccgacatgacgaagt

SEQ ID NO: 306 - DNA, 20 bp, Artificial Sequence
  Description of Artificial Sequence: Synthetic oligonucleotide
gatcagcccatcaaatgagg

SEQ ID NO: 307 - DNA, 20 bp, Artificial Sequence
  Description of Artificial Sequence: Synthetic oligonucleotide
cccagcgagtgaacacggca

SEQ ID NO: 308 - DNA, 20 bp, Artificial Sequence
  Description of Artificial Sequence: Synthetic oligonucleotide
accttgtctgcctccacagg

SEQ ID NO: 309 - DNA, 20 bp, Artificial Sequence
  Description of Artificial Sequence: Synthetic oligonucleotide
catcaacgccaacgtgcagg

SEQ ID NO: 310 - DNA, 20 bp, Artificial Sequence
  Description of Artificial Sequence: Synthetic oligonucleotide
gtaaaagatgtgcatttgag

SEQ ID NO: 311 - DNA, 20 bp, Artificial Sequence
  Description of Artificial Sequence: Synthetic oligonucleotide
atgtcgttgagcaagtttac

SEQ ID NO: 312 - DNA, 20 bp, Artificial Sequence
  Description of Artificial Sequence: Synthetic oligonucleotide
caccatgaagggccacgcgt

SEQ ID NO: 313 - DNA, 20 bp, Artificial Sequence
  Description of Artificial Sequence: Synthetic oligonucleotide
cagtgcctggccatgggctg

SEQ ID NO: 314 - DNA, 20 bp, Artificial Sequence
  Description of Artificial Sequence: Synthetic oligonucleotide
gacactacattacaaacaac

SEQ ID NO: 315 - DNA, 20 bp, Artificial Sequence
  Description of Artificial Sequence: Synthetic oligonucleotide
gcctgcttccatgcctggtt

SEQ ID NO: 316 - DNA, 20 bp, Artificial Sequence
  Description of Artificial Sequence: Synthetic oligonucleotide
ggacgaaggaggcaattccg

SEQ ID NO: 317 - DNA, 20 bp, Artificial Sequence
  Description of Artificial Sequence: Synthetic oligonucleotide
ctgcctccacagggggactc

SEQ ID NO: 318 - DNA, 20 bp, Artificial Sequence
  Description of Artificial Sequence: Synthetic oligonucleotide
tgcgcccaacttcgtcatgt

SEQ ID NO: 319 - DNA, 20 bp, Artificial Sequence
  Description of Artificial Sequence: Synthetic oligonucleotide
tgccagatgctggagagtgt

SEQ ID NO: 320 - DNA, 20 bp, Artificial Sequence
  Description of Artificial Sequence: Synthetic oligonucleotide
cacctgcacgttggcgttga

SEQ ID NO: 321 - DNA, 20 bp, Artificial Sequence
  Description of Artificial Sequence: Synthetic oligonucleotide
cggaattgcctccttcgtcc

SEQ ID NO: 322 - DNA, 20 bp, Artificial Sequence
  Description of Artificial Sequence: Synthetic oligonucleotide
cccactgagaagggctgccc

SEQ ID NO: 323 - DNA, 20 bp, Artificial Sequence
  Description of Artificial Sequence: Synthetic oligonucleotide
tttcctcgcctgtgtcctgc

SEQ ID NO: 324 - DNA, 20 bp, Artificial Sequence
  Description of Artificial Sequence: Synthetic oligonucleotide
ctgcacgttggcgttgatgg

SEQ ID NO: 325 - DNA, 20 bp, Artificial Sequence
  Description of Artificial Sequence: Synthetic oligonucleotide
ggcgcagcctccccggacga

SEQ ID NO: 326 - DNA, 20 bp, Artificial Sequence
  Description of Artificial Sequence: Synthetic oligonucleotide
gtgtgtgtagcaccgcgtaa

SEQ ID NO: 327 - DNA, 20 bp, Artificial Sequence
  Description of Artificial Sequence: Synthetic oligonucleotide
tcccagcacagggctataag

SEQ ID NO: 328 - DNA, 20 bp, Artificial Sequence
  Description of Artificial Sequence: Synthetic oligonucleotide
cccaccttgtctgcctccac

SEQ ID NO: 329 - DNA, 20 bp, Artificial Sequence
  Description of Artificial Sequence: Synthetic oligonucleotide
gtcttgactgctagaatgta

SEQ ID NO: 330 - DNA, 20 bp, Artificial Sequence
  Description of Artificial Sequence: Synthetic oligonucleotide
gtttacaaactgtgccaccg

SEQ ID NO: 331 - DNA, 20 bp, Artificial Sequence
  Description of Artificial Sequence: Synthetic oligonucleotide
cggtggcacagtttgtaaac

SEQ ID NO: 332 - DNA, 20 bp, Artificial Sequence
  Description of Artificial Sequence: Synthetic oligonucleotide
tctcgcggcgggagcccacc

SEQ ID NO: 333 - DNA, 20 bp, Artificial Sequence
  Description of Artificial Sequence: Synthetic oligonucleotide
agacgttgctgcgacggcag

SEQ ID NO: 334 - DNA, 20 bp, Artificial Sequence
  Description of Artificial Sequence: Synthetic oligonucleotide
gtactatgttaacgtatttc

SEQ ID NO: 335 - DNA, 20 bp, Artificial Sequence
  Description of Artificial Sequence: Synthetic oligonucleotide
tgtcctgccggccttgctgc

SEQ ID NO: 336 - DNA, 20 bp, Artificial Sequence
  Description of Artificial Sequence: Synthetic oligonucleotide
cgctggcctcggagattgtg

SEQ ID NO: 337 - DNA, 20 bp, Artificial Sequence
  Description of Artificial Sequence: Synthetic oligonucleotide
acaccatgaagggccacgcg

SEQ ID NO: 338 - DNA, 20 bp, Artificial Sequence
  Description of Artificial Sequence: Synthetic oligonucleotide
tccccaccgccacagctcaa

SEQ ID NO: 339 - DNA, 20 bp, Artificial Sequence
  Description of Artificial Sequence: Synthetic oligonucleotide
gtccaagaacaaagcaaaga

SEQ ID NO: 340 - DNA, 20 bp, Artificial Sequence
  Description of Artificial Sequence: Synthetic oligonucleotide
cagcaaggccggcaggacac

SEQ ID NO: 341 - DNA, 20 bp, Artificial Sequence
  Description of Artificial Sequence: Synthetic oligonucleotide
agtttacaaactgtgccacc

SEQ ID NO: 342 - DNA, 20 bp, Artificial Sequence
  Description of Artificial Sequence: Synthetic oligonucleotide
accgccacagctcaacgggt

SEQ ID NO: 343 - DNA, 20 bp, Artificial Sequence
  Description of Artificial Sequence: Synthetic oligonucleotide
gtctgcaacgggctaatcca

SEQ ID NO: 344 - DNA, 20 bp, Artificial Sequence
  Description of Artificial Sequence: Synthetic oligonucleotide
ctaccccgatgcctttgccc

SEQ ID NO: 345 - DNA, 20 bp, Artificial Sequence
  Description of Artificial Sequence: Synthetic oligonucleotide
gatgtcgttgagcaagttta

SEQ ID NO: 346 - DNA, 20 bp, Artificial Sequence
  Description of Artificial Sequence: Synthetic oligonucleotide
cccagccccaccatgaccct

SEQ ID NO: 347 - DNA, 20 bp, Artificial Sequence
  Description of Artificial Sequence: Synthetic oligonucleotide
gcaacgtctgcactctcgtg

SEQ ID NO: 348 - DNA, 20 bp, Artificial Sequence
  Description of Artificial Sequence: Synthetic oligonucleotide
tgccaccggggcaaaggcat

SEQ ID NO: 349 - DNA, 20 bp, Artificial Sequence
  Description of Artificial Sequence: Synthetic oligonucleotide
caccatcaacgccaacgtgc

SEQ ID NO: 350 - DNA, 20 bp, Artificial Sequence
  Description of Artificial Sequence: Synthetic oligonucleotide
ggcatcggggtagagccctg

SEQ ID NO: 351 - DNA, 20 bp, Artificial Sequence
  Description of Artificial Sequence: Synthetic oligonucleotide
ccgttgcattgccccacggc

SEQ ID NO: 352 - DNA, 20 bp, Artificial Sequence
  Description of Artificial Sequence: Synthetic oligonucleotide
cagtttacaaactgtgccac

SEQ ID NO: 353 - DNA, 20 bp, Artificial Sequence
  Description of Artificial Sequence: Synthetic oligonucleotide
gattagcccgttgcagacca

SEQ ID NO: 354 - DNA, 20 bp, Artificial Sequence
  Description of Artificial Sequence: Synthetic oligonucleotide
cgaggaaaagacacgcgagt

SEQ ID NO: 355 - DNA, 20 bp, Artificial Sequence
  Description of Artificial Sequence: Synthetic oligonucleotide
acggaattgcctccttcgtc

SEQ ID NO: 356 - DNA, 20 bp, Artificial Sequence
  Description of Artificial Sequence: Synthetic oligonucleotide
gccaccggggcaaaggcatc

SEQ ID NO: 357 - DNA, 20 bp, Artificial Sequence
  Description of Artificial Sequence: Synthetic oligonucleotide
tgcaacggcctcccagcaca

SEQ ID NO: 358 - DNA, 20 bp, Artificial Sequence
  Description of Artificial Sequence: Synthetic oligonucleotide
aacgtctgcactctcgtgag

SEQ ID NO: 359 - DNA, 20 bp, Artificial Sequence
  Description of Artificial Sequence: Synthetic oligonucleotide
caacccctgtccccaccccc

SEQ ID NO: 360 - DNA, 20 bp, Artificial Sequence
  Description of Artificial Sequence: Synthetic oligonucleotide
gagtgcagacgttgctgcga

SEQ ID NO: 361 - DNA, 20 bp, Artificial Sequence
  Description of Artificial Sequence: Synthetic oligonucleotide
tccgaggccagcgcggtgcc

SEQ ID NO: 362 - DNA, 20 bp, Artificial Sequence
  Description of Artificial Sequence: Synthetic oligonucleotide
gccaggggagaggaagtgga

SEQ ID NO: 363 - DNA, 20 bp, Artificial Sequence
  Description of Artificial Sequence: Synthetic oligonucleotide
agtgtgggtgtgggcagctg

SEQ ID NO: 364 - DNA, 20 bp, Artificial Sequence
  Description of Artificial Sequence: Synthetic oligonucleotide
gggagccctgggcaggttca

SEQ ID NO: 365 - DNA, 20 bp, Artificial Sequence
  Description of Artificial Sequence: Synthetic oligonucleotide
ccccaccgccacagctcaac

SEQ ID NO: 366 - DNA, 20 bp, Artificial Sequence
  Description of Artificial Sequence: Synthetic oligonucleotide
tgccaggggagaggaagtgg

SEQ ID NO: 367 - DNA, 20 bp, Artificial Sequence
  Description of Artificial Sequence: Synthetic oligonucleotide
attagcccgttgcagaccaa

SEQ ID NO: 368 - DNA, 20 bp, Artificial Sequence
  Description of Artificial Sequence: Synthetic oligonucleotide
ttagcccgttgcagaccaag

SEQ ID NO: 369 - DNA, 20 bp, Artificial Sequence
  Description of Artificial Sequence: Synthetic oligonucleotide
ctgtgccaggggagaggaag

SEQ ID NO: 370 - DNA, 20 bp, Artificial Sequence
  Description of Artificial Sequence: Synthetic oligonucleotide
gccctccacttcctctcccc

SEQ ID NO: 371 - DNA, 20 bp, Artificial Sequence
  Description of Artificial Sequence: Synthetic oligonucleotide
caacgtctgcactctcgtga

SEQ ID NO: 372 - DNA, 20 bp, Artificial Sequence
  Description of Artificial Sequence: Synthetic oligonucleotide
attgcctccttcgtccgggg

SEQ ID NO: 373 - DNA, 20 bp, Artificial Sequence
  Description of Artificial Sequence: Synthetic oligonucleotide
atggtggggctggggctccg

SEQ ID NO: 374 - skipped

SEQ ID NO: 375 - protein, 267 aa, Homo sapiens
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVAQFVNWIDSIIQRSEDNPCPHPRDPDPASRTH

SEQ ID NO: 376 - protein, 190 aa, Artificial Sequence
  Description of Artificial Sequence: Synthetic polypeptide
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLSAVAATSALS

SEQ ID NO: 377 - protein, 211 aa, Artificial Sequence
  Description of Artificial Sequence: Synthetic polypeptide
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAPASVSGTPAAPWSATG

SEQ ID NO: 378 - protein, 238 aa, Artificial Sequence
  Description of Artificial Sequence: Synthetic polypeptide
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHELPPSSGEAAPQGSTPMPLPRWHSL

SEQ ID NO: 379 - protein, 238 aa, Artificial Sequence
  Description of Artificial Sequence: Synthetic polypeptide
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFGDSGSPLVCNGLIHGIASFVRGGCASGLYPDAFAPVARL

SEQ ID NO: 380 - protein, 267 aa, Artificial Sequence
  Description of Artificial Sequence: Synthetic polypeptide
MTLGRRLACLFLACVLPALLLGGTALASEIVGGRRARPHAWPFMVSLQLRGGHFCGATLIAPNFVMSAAHCVANVNVRAVRVVLGAHNLSRREPTRQVFAVQRIFENGYDPVNLLNDIVILQLNGSATINANVQVAQLPAQGRRLGNGVQCLAMGWGLLGRNRGIASVLQELNVTVVTSLCRRSNVCTLVRGRQAGVCFRGLRQPLGLQRANPRNCLLRPGRLRLRALPRCLCPGGTVCKLDRLYHPTLRGQPLSPPPGPGPGQQDP

SEQ ID NO: 381 - DNA, 20 bp, Artificial Sequence
  Description of Artificial Sequence: Synthetic oligonucleotide
ccggccccccacaatctccg

SEQ ID NO: 382 - DNA, 20 bp, Artificial Sequence
  Description of Artificial Sequence: Synthetic oligonucleotide
ttgtggggggccggcgagcg

SEQ ID NO: 383 - DNA, 20 bp, Artificial Sequence
  Description of Artificial Sequence: Synthetic oligonucleotide
catgtcggccgcgcactgcg

SEQ ID NO: 384 - DNA, 20 bp, Artificial Sequence
  Description of Artificial Sequence: Synthetic oligonucleotide
cgcgctggcctcggagattg

SEQ ID NO: 385 - DNA, 810 bp, Homo sapiens
cccaccatgaccctcggccgccgactcgcgtgtcttttcctcgcctgtgtcctgccggccttgctgctggggggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcgtggcgaatgtaaacgtccgcgcggtgcgggtggtcctgggagcccataacctctcgcggcgggagcccacccggcaggtgttcgccgtgcagcgcatcttcgaaaacggctacgaccccgtaaacttgctcaacgacatcgtgattctccagctcaacgggtcggccaccatcaacgccaacgtgcaggtggcccagctgccggctcagggacgccgcctgggcaacggggtgcagtgcctggccatgggctggggccttctgggcaggaaccgtgggatcgccagcgtcctgcaggagctcaacgtgacggtggtgacgtccctctgccgtcgcagcaacgtctgcactctcgtgaggggccggcaggccggcgtctgtttcggggactccggcagccccttggtctgcaacgggctaatccacggaattgcctccttcgtccggggaggctgcgcctcagggctctaccccgatgcctttgccccggtggcacagtttgtaaactggatcgactctatcatccaacgctccgaggacaacccctgtccccacccccgggacccggacccggccagcaggacccactga

SEQ ID NO: 386 - DNA, 582 bp, Artificial Sequence
  Description of Artificial Sequence: Synthetic polynucleotide
cctcagtttcctcatctgaacaacaggggtgcgaacggccccgatcccgtgggttcccggtgggggatcccgtgggttcccggtgggggatcccgtgggttcctggtgggggatccagaggccccgtggccgggaggggacaggctccttggcaggcactcagcacccgcacccggtgtgtccccaggcaccgcgctggcctcggagattgtggggggccggcgagcgcggccccacgcgtggcccttcatggtgtccctgcagctgcgcggaggccacttctgcggcgccaccctgattgcgcccaacttcgtcatgtcggccgcgcactgcggggaggctgcgcctcagggctctaccccgatgcctttgccccggtggcacagtttgtaaactggatcgactctatcatccaacgctccgaggacaacccctgtccccacccccgggacccggacccggccagcaggacccactgagaagggctgcccgggtcacctcagctgcccacacccacactctccagcatctggcacaataaacattctctgttttgtagaatgtgtttgatgctccttggct

SEQ ID NO: 387 - protein, 7 aa, Simian virus 40
PKKKRKV

SEQ ID NO: 388 - protein, 16 aa, Unknown
  Description of Unknown: Nucleoplasmin bipartite NLS sequence
KRPAATKKAGQAKKKK

SEQ ID NO: 389 - protein, 9 aa, Unknown
  Description of Unknown: C-myc NLS sequence
PAAKRVKLD

SEQ ID NO: 390 - protein, 11 aa, Unknown
  Description of Unknown: C-myc NLS sequence
RQRRNELKRSP

SEQ ID NO: 391 - protein, 38 aa, Homo sapiens
NQSSNFGPMKGGNFGGRSSGPYGGGGQYFAKPRNQGGY

SEQ ID NO: 392 - protein, 42 aa, Unknown
  Description of Unknown: IBB domain from importin-alpha sequence
RMRIZFKNKGKDTAELRRRRVEVSVELRKAKKDEQILKRRNV

SEQ ID NO: 393 - protein, 8 aa, Unknown
  Description of Unknown: Myoma T protein sequence
VSRKRPRP

SEQ ID NO: 394 - protein, 8 aa, Unknown
  Description of Unknown: Myoma T protein sequence
PPKKARED

SEQ ID NO: 395 - protein, 8 aa, Homo sapiens
PQPKKKPL

SEQ ID NO: 396 - protein, 12 aa, Mus musculus
SALIKKKKKMAP

SEQ ID NO: 397 - protein, 5 aa, Influenza virus
DRLRR

SEQ ID NO: 398 - protein, 7 aa, Influenza virus
PKQKKRK

SEQ ID NO: 399 - protein, 10 aa, Unknown
  Description of Unknown: Hepatitis delta virus
RKLKKKIKKL

SEQ ID NO: 400 - protein, 10 aa, Mus musculus
REKKKFLKRR

SEQ ID NO: 401 - protein, 20 aa, Homo sapiens
KRKGDEVDGVDEVAKKKSKK

SEQ ID NO: 402 - protein, 17 aa, Homo sapiens
RKCLQAGMNLEARKTKK

SEQ ID NO: 403 - protein, 21 aa, Unknown
  Description of Unknown: 53BP1 NLS sequence
GKRKLITSEEERSPAKRGRKS

SEQ ID NO: 404 - protein, 6 aa, Unknown
  Description of Unknown: BRCA1 NLS sequence
KRKRRP

SEQ ID NO: 405 - protein, 20 aa, Unknown
  Description of Unknown: SRC-1 NLS sequence
KRKGSPCDTLASSTEKRRRE

SEQ ID NO: 406 - protein, 12 aa, Unknown
  Description of Unknown: IRF3 NLS sequences
KRNFRSALNRKE

SEQ ID NO: 407 - protein, 9 aa, Unknown
  Description of Unknown: “LAGLIDADG” family motif
LAGLIDADG

SEQ ID NO: 408 - protein, 6 aa, Artificial Sequence
  Description of Artificial Sequence: Synthetic 6xHis tag
HHHHHH

SEQ ID NO: 409 - DNA, 74 bp, Artificial Sequence
  Description of Artificial Sequence: Synthetic oligonucleotide
ggggactccggcagccccttggtctgcaacgggctaatccacggaattgcctccttcgtccggggaggctgcgc

SEQ ID NO: 410 - DNA, 20 bp, Artificial Sequence
  Description of Artificial Sequence: Synthetic oligonucleotide
cagccccttgtcgtccgggg

SEQ ID NO: 411 - DNA, 20 bp, Artificial Sequence
  Description of Artificial Sequence: Synthetic oligonucleotide
cagccccttggggaggctgc

SEQ ID NO: 412 - DNA, 31 bp, Artificial Sequence
  Description of Artificial Sequence: Synthetic oligonucleotide
accgcgctggcctcggagtttgcaacggaat